(19) United States
(12) Patent Application Publication
(10) Pub. No.: US 2015/0329821 A1
(43) Pub. Date: Nov. 19, 2015

(54) METHODS OF DIFFERENTIATING STEM CELLS INTO ONE OR MORE CELL LINEAGES

(71) Applicant: AGENCY FOR SCIENCE, TECHNOLOGY AND RESEARCH, Singapore (SG)

(72) Inventors: Lay Teng ANG, Singapore (SG); Kyle M. LOH, Singapore (SG); Bing LIM, Singapore (SG)

(21) Appl. No.: 14/437,147

(22) PCT Filed: Oct. 21, 2013
(86) PCT No.: PCT/SG2013/000453

§ 371 Date: Apr. 20, 2015

(30) Foreign Application Priority Data
Oct. 19, 2012 (SG) 201207832-5

(51) Int. Cl. C12N 5/0735; C12N 5/074

(52) U.S. Cl. CPC C12N 5/0606; C12N 5/0696; C12N 2506/02; C12N 2506/45; C12N 2501/998

(57) ABSTRACT

The present disclosure provides an understanding of the regulation of the developmental phases of stem cells and their induction into relevant cell lineages, such as primitive streak, endoderm, mesoderm, or subterrotories of endoderm's. In particular, the present disclosure provides methods, culture medium and kits for the maintenance and differentiation of stem cells into relevant cell lineages.

### CROSS-REFERENCE TO RELATED APPLICATIONS

This application claims the benefit of priority of Singapore application No. 201207832-5, filed 19 Oct. 2012, the contents of it being hereby incorporated by reference in its entirety for all purposes.

### FIELD OF THE INVENTION

The present invention relates generally to the field of biotechnology. In particular, the present invention relates to methods for differentiating a pluripotent stem cell or pluripotent cell into multiple cell lineages. The present invention further relates to culture mediums and kits for use in performing the methods as described herein.

### BACKGROUND OF THE INVENTION

At multiple developmental junctures, lineage-specifying transcription factors (TFs) direct multipotent progenitors towards a single lineage outcome and repress alternate fates, ensuring a unilateral lineage decision (Graf and Enver, 2009; Loh and Lim, 2011). However, the extrinsic signals that govern such lineage bifurcations and the precise cell types they specify remain to be fully clarified, despite informative insights gained from in vivo genetic perturbations (Schier and Talbot, 2005; Tam and Loebel, 2007) and ex vivo explant approaches (e.g., Bernardo et al., 2011; Deutsch et al., 2001). Pertinent issues include the sequence and timings of dynamic signaling switches that drive successive cell-fate transitions (Wandzioch and Zaret, 2009) and how alternate lineages are segregated at each branchpoint. Therefore knowledge of signals that preside over early lineage bifurcations is of great benefit in differentiating pluripotent stem cells such as human pluripotent stem cells (hPSC) into committed cell-types for cell replacement therapy (Cohen and Melton, 2011; McKnight et al., 2010; Murry and Keller, 2008; Smith, 2001).

In this regard, there is a need to further investigate the signaling dynamics that drive induction and anterior-posterior patterning of the definitive endoderm (DE) germ layer and subsequent organ formation. DE is the embryonic precursor to organs including the thyroid, lungs, pancreas, liver and intestines (\check{S}vajger and Levak-\check{S}vajger, 1974). The pluripotent epiblast (E5.5 in mouse embryogenesis) differentiates into the anterior primitive streak (˜E6.5), which generates DE (˜E7.0-E7.5) (Lawson et al., 1991; Tam and Beddington, 1987). DE is then patterned along the anterior-posterior axis into distinct foregut, midgut and hindgut territories (˜E8.5) and endoderm organ primordia arise from specific anteroposterior domains (˜E9.5) (Zorn and Wells, 2009).

Various methods to differentiate pluripotent stem cells, such as hPSC, towards DE employ animal serum, feeder co-culture or defined conditions (Cheng et al., 2012; D'Amour et al., 2005; Touboul et al., 2010) but typically yield a mixture of DE and other contaminating lineages, with induction efficiencies fluctuating between cell lines (Cohen and Melton, 2011; McKnight et al., 2010; Smith, 2001). Admixed early DE populations harboring contaminating lineages complicate the subsequent generation of endodermal organ derivatives (McKnight et al., 2010). In vertebrate embryos and during PSC differentiation, Nodal/TGFβ/Activin signaling is imperative for DE specification whereas BMP broadly induces mesodermal subtypes (e.g., Bernardo et al., 2011; D'Amour et al., 2005; Dunn et al., 2004). Yet TGFβ signaling (even with additional factors) is insufficient to specify homogeneous DE (quantified by Chetty et al., 2013). BMP, FGF, VEGF and Wnt have also been employed together with TGFβ signals to generate DE (Cheng et al., 2012; Goldman et al., 2013; Green et al., 2011; Kroon et al., 2008; Nostro et al., 20111 Touboul et al., 2010). However, these morphogens are also implicated in mesoderm commitment (Davis et al., 2008; Gertow et al., 2013) and their precise involvement in DE induction remains to be clarified.

There is currently no coherent understanding of the signaling logic underlying multiple steps of induction and patterning of the germ layers and differentiation into the various cell lineages.

It is an aim of the present invention to elucidate the underlying signaling logic of stem cell induction and differentiation in order to unilaterally drive stem cells to a single cell fate with minimal extraneous lineages.

### SUMMARY OF THE INVENTION

According to one aspect there is provided a method of differentiating stem cells into one or more cell lineages comprising contacting said cells with one or more activators of TGFβ/Nodal signaling and one or more activators of Wnt signaling.

According to another aspect there is provided a method of differentiating anterior primitive streak cells into cells of definitive endoderm (DE) lineage, by contacting said cells of the anterior primitive streak lineage with one or more activators of TGFβ/Nodal signaling, and one or more inhibitors of BMP signaling, or one or more inhibitors of Wnt signaling.

According to another aspect there is provided a method of differentiating cells of the DE into cells of the AFG, by contacting said DE cells with a TGFβ inhibitor and a BMP inhibitor.

According to another aspect there is provided a method of differentiating cells of the DE lineage into cells of the PFG, by contacting said cells of the DE with retinoic acid, a BMP inhibitor, a Wnt inhibitor and a FGF/MAPK inhibitor.

According to another aspect there is provided a method of differentiating cells of the DE lineage into cells of the MHG, by contacting said DE cells with a BMP activator, a Wnt activator and an FGF activator.

According to another aspect there is provided a method of inducing pancreatic progenitors of the PFG from the DE within three days by contacting said PFG with one or more FGF/MAPK inhibitors; one or more BMP inhibitors; and retinoic acid (RA.

According to another aspect there is provided a method of inducing liver progenitors of the PFG from the DE within four days by contacting said PFG with one or more TGFβ inhibitors; one or more BMP activators, retinoic acid and one or more Wnt inhibitors.

According to another aspect there is provided a cell culture medium for differentiating a stem cell into one or more cell lineages comprising one or more of the following factors: one or more FGF/MAPK inhibitors; one or more Hedgehog inhibitors; one or more. BMP inhibitors, one or more Wnt inhibitors, retinoic acid, Activin A, and one- or more inhibitors of PI3K/mTOR signaling.

According to another aspect there is provided a cell culture medium for differentiating a stem cell into one or more cell lineages comprising one or more of the following factors: one or more activators of TGFβ/Nodal signaling, one or more activators of Wnt/β-catenin signaling, and one or more inhibitors of PI3K/mTOR signaling.

According to another aspect there is provided a cell culture medium for differentiating a stem cell into one or more cell lineages comprising one or more of the following factors: one or more activators of TGFβ/Nodal signaling, and one or more inhibitors of BMP signaling.

According to another aspect there is provided a cell culture medium for differentiating a stem cell into one or more cell lineages comprising a TGFβ inhibitor and a BMP inhibitor.

According to another aspect there is provided a cell culture medium for differentiating a stem cell into one or more cell lineages comprising one or more of the following factors: retinoic acid, a BMP inhibitor, a Wnt inhibitor and a FGF/MAPK inhibitor.

According to another aspect there is provided a cell culture medium for differentiating a stem cell into one or more cell lineages comprising one or more of the following factors: BMP4, a Wnt activator and an FGF activator.

According to another aspect there is provided a cell culture medium for differentiating a stem cell into one or more cell lineages comprising one or more of the following factors: one or more FGF/MAPK inhibitors; one or more Hedgehog inhibitors; one or more BMP inhibitors; one or more WNT inhibitors; retinoic acid (RA), and Activin A.

According to another aspect there is provided a cell culture medium for differentiating a stem cell into one or more cell lineages comprising one or more of the following factors: one or more TGFβ inhibitors; one or more BMP activators, and one or more Wnt inhibitors.

According to another aspect there is provided a cell produced according to any of the methods as described herein.

According to another aspect there is provided a kit for use in any of the methods described herein, comprising one or more containers of cell culture medium as described herein, together with instructions for use.

### DETAILED DESCRIPTION OF THE PRESENT INVENTION

### Definitions

The following words and terms used herein shall have the meaning indicated:

As used herein, the term “stem cells” include but are not limited to undifferentiated cells defined by their ability at the single cell level to both self-renew and differentiate to produce progeny cells, including self-renewing progenitors, non-renewing progenitors, and terminally differentiated cells. For example, “stem cells” may include (1) totipotent stem cells; (2) pluripotent stem cells; (3) multipotent stem cells; (4) oligopotent stem cells; and (5) unipotent stem cells.

As used herein, the term “totipotency” refers to a cell with a developmental potential to make all of the cells in the adult body as well as the extra-embryonic tissues, including the placenta. The fertilized egg (zygote) is totipotent, as are the cells (blastomeres) of the morula (up to the 16-cell stage following fertilization).

As used herein, the term “pluripotent stem cell” refers to a cell with the developmental potential, under different conditions, to differentiate to cell types characteristic of all three germ cell layers, i.e., endoderm (e.g., gut tissue), mesoderm (including blood, muscle, and vessels), and ectoderm (such as skin and nerve). The developmental competency of a cell to differentiate to all three germ layers can be determined using, for example, a nude mouse teratoma formation assay. In some embodiments, pluripotency can also be evidenced by the expression of embryonic stem (ES) cell markers, although the preferred test for pluripotency of a cell or population of cells generated using the compositions and methods described herein is the demonstration that a cell has the developmental potential to differentiate into cells of each of the three germ layers.

As used herein, the term “induced pluripotent stem cells” or, iPSCs, means that the stem cells are produced from differentiated adult cells that have been induced or changed, i.e., reprogrammed into cells capable of differentiating into tissues of all three germ or dermal layers: mesoderm, endoderm, and ectoderm. The iPSCs produced do not refer to cells as they are found in nature.

As used herein, the term “embryonic stem cell” refers to naturally occurring pluripotent stem cells of the inner cell mass of the embryonic blastocyst. Such cells can similarly be obtained from the inner cell mass of blastocysts derived from somatic cell nuclear transfer. Embryonic stem cells are pluripotent and give rise during development to all derivatives of the three primary germ layers: ectoderm, endoderm and mesoderm. In other words, they can develop into each of the more than 200 cell types of the adult body when given sufficient and necessary stimulation for a specific cell type. They do not contribute to the extra-embryonic membranes or the placenta, i.e., are not totipotent.

As used herein, the term “multipotent stem cell” refers to a cell that has the developmental potential to differentiate into cells of one or more germ layers, but not all three. Thus, a multipotent cell can also be termed a “partially differentiated cell.” Multipotent cells are well known in the art, and examples of multipotent cells include adult stem cells, such as for example, hematopoietic stem cells and neural stem cells. “Multipotent” indicates that a cell may form many types of cells in a given lineage, but not cells of other lineages. For example, a multipotent hematopoietic cell can form the many different types of blood cells (red, white, platelets, etc.), but it cannot form neurons. Accordingly, the term “multipotency” refers to a state of a cell with a degree of developmental potential that is less than totipotent and pluripotent.

As used herein, the term “Differentiation” is the process by which an unspecialized (“uncommitted”) or less specialized cell acquires the features of a specialized cell such as, for example, a nerve cell or a muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. De-differentiation refers to the process by which a cell reverts to a less specialized (or committed) position within the lineage of a cell. As used herein, the lineage of a cell defines the heredity of the cell, i.e., which cells it came from and what cells it can give rise to. The lineage of a cell places the cell within a hereditary scheme of development and differentiation. A lineage-specific marker refers to a characteristic specifically associated with the phenotype of cells of a lineage of interest and can be used to assess the differentiation of an uncommitted cell to the lineage of interest.

As used herein, the term “undifferentiated cell” refers to a cell in an undifferentiated state that has the property of self-renewal and has the developmental potential to differentiate into multiple cell types, without a specific implied meaning regarding developmental potential (i.e., totipotent, pluripotent, multipotent, etc.).

As used herein, the term “progenitor cell” refers to cells that have greater developmental potential, i.e., a cellular phenotype that is more primitive (e.g., is at an earlier step along a developmental pathway or progression) relative to a cell which it can give rise to by differentiation. Often, progenitor cells have significant or very high proliferative potential. Progenitor cells can give rise to multiple distinct cells having lower developmental potential, i.e., differentiated cell types, or to a single differentiated cell type, depending on the developmental pathway and on the environment in which the cells develop and differentiate.

As used herein, the term “Markers” refers to nucleic acid or polypeptide molecule that is differentially expressed in a cell of interest. In this context, differential expression means an increased level for a positive marker and a decreased level for a negative marker. The detectable level of the marker nucleic acid or polypeptide is sufficiently higher or lower in the cells of interest compared to other cells, such that the cell of interest can be identified and distinguished from other cells using any of a variety of methods known in the art.

As used herein, the term “modulator” in the context of TGFβ/Nodal signaling, Wnt signaling, PI3K/mTOR signaling, BMP signaling, growth factor signaling, or activity of retinoic acid, FGF/MAPK, Hedgehog, refers to any molecule or compound which either enhances or inhibits the biological activity of the defined signaling pathway or its target. The inhibitors or activators may include but are not limited to peptides, antibodies, or small molecules that target the receptors, transcription factors, signaling mediators/transducers and the like that are a part of the signaling pathway or the targets natural ligand thereby modulating the biological activity of the signaling pathways. In this regard, as used herein “inhibitors” or “activators” in the context of TGFβ/Nodal signaling, Wnt signaling, PI3K/mTOR signaling, BMP signaling, growth factor signaling, or activity of retinoic acid, FGF/MAPK or Hedgehog, refers to the inhibition or activation of one or more components of the defined signaling, including but not limited to the signaling ligands, receptors, transducers, signaling mediators and transcriptional factors. In particular, “inhibitors” or “activators” may refer to antagonists or agonists of the ligand protein of the signaling pathways or any component of the signaling transduction pathways besides the ligand protein, (e.g. the receptors, transducers, signaling mediators)

As used herein the phrase “culture medium” refers to a liquid substance used to support the growth of stem cells and any of the cell lineages. The culture medium used by the invention according to some embodiments can be a liquid-based medium, for example water, which may comprise a combination of substances such as salts, nutrients, minerals, vitamins, amino acids, nucleic acids, proteins such as cytokines, growth factors and hormones.

As used herein, the term “feeder cell” refers to feeder cells (e.g., fibroblasts) that maintain stem cells in a proliferative state when the stem cells are co-cultured on the feeder cells or when the pluripotent stem cells are cultured on a matrix (e.g., an extracellular matrix, a synthetic matrix) in the presence of a conditioned medium generated by the feeder cells. The support of the feeder cells depends on the structure of the feeder cells while in culture (e.g., the three dimensional matrix formed by culturing the feeder cells in a tissue culture plate), function of the feeder cells (e.g., the secretion of growth factors, nutrients and hormones by the feeder cells, the growth rate of the feeder cells, the expansion ability of the feeder cells before senescence) and/or the attachment of the stem cells to the feeder cell layer(s).

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### Disclosure of Optional Embodiments

Before the present inventions are described, it is to be understood that this invention is not limited to particular embodiments described, as such may vary. It is also to be understood that the terminology used herein is for purposes of describing particular embodiments only, and is not intended to be limiting, since the scope of the present invention will be limited only in the appended claims.

Unless defined otherwise, all technical and scientific terms used herein have the same meaning as commonly understood by one of ordinary skill in the art to which this invention belongs. Any methods and materials similar or equivalent to those described herein can be used in the practice or testing of the invention, as it will be understood that modifications and variations are encompassed within the spirit and scope of the instant disclosure.

The present disclosure and embodiments relate to the methods of separating mutually-exclusive cell lineages at 4 consecutive steps of endoderm development, in relation to primitive streak (PS) induction; segregation of endoderm versus mesoderm germ layers; endoderm anterior-posterior patterning; and bifurcation of the cell lineages. In particular, the present disclosure and embodiments relate to the determination of which signals instructed or repressed specific developmental outcomes at each endodermal bifurcation that enabled homogeneous hPSC differentiation down one path or the other. Advantageously, the present disclosure provides the knowledge of precise temporal signaling dynamics, combined with efficient differentiation throughout successive developmental steps, culminated in a single strategy to universally differentiate diverse hPSC lines into pure populations of various endoderm cell types by excluding alternate lineages at each branchpoint.

Accordingly, the present invention provides methods of differentiating stem cells into one or more cell lineages. In this regard, the stem cells may include but are not limited to totipotent stem cells, pluripotent stem cells, multipotent stem cells, oligopotent stem cells, or unipotent stem cells.

In one embodiment, the stem cells may be pluripotent stem cells including but not limited to the human embryonic stem cell (hESC), which may or may not be derived from a human embryonic source. For example Pluripotent stem cells suitable for use in the present invention may include but are not limited to human embryonic stem cell line H9 (NIH code: WA09), the human embryonic stem cell line Hl (NIH code: WAOl), the human embryonic stem cell line H7 (NIH code: WA07), the human embryonic stem cell line SA002 (Cellartis, Sweden), Hes3 (NIH code: ES03), MeL1 (NIH code: 0139), or stem cells that express at least one of the following markers characteristic of pluripotent cells: ABCG2, cripto, CD9, FoxD3, Connexin43, Connexin45, Oct4, Sox2, Nanog, hTERT, UTF-I, ZFP42, SSEA-3, SSEA-4, Tral-60, Tral-81. Similarly, the pluripotent stem cell may be an induced pluripotent stem (iPS) cell, which may be derived from non-embryonic sources, and can proliferate without limit and differentiate into each of the three embryonic germ layers. For example an IPS cell line can include but is not limited to BJC1 and BJC3. It is understood that iPS cells behave in culture essentially the same as ESCs.

In this regard, as is well-known in the context of the technical field, pluripotent stem cells may differentiate into functional cells of various cell lineages from the multiple germ layers of either endoderm, mesoderm or ectoderm, as well as to give rise to tissues of multiple germ layers following transplantation and to contribute substantially to most, if not all, tissues following injection into blastocysts. For example, the pluripotent stem cells may be differentiated into cell lineages of the endoderm that may include but are not limited to the anterior primitive streak (APS) lineage, definitive endoderm (DE) lineage, anterior foregut (AFG) lineage, posterior foregut (PFG) lineage, mid gut hind (MHG) lineage, pancreatic progenitor lineage, or hepatacytic progenitor lineage. Alternatively, the pluripotent stem cells may be differentiated into cell lineages of the mesoderm that include but are not limited to cardiac, lateral plate, paraxial, pre-somitic, somitic mesoderm, intermediate and extra-embryonic mesoderm, or differentiated into cell lineages of the ectoderm that include but are not limited to neuroectoderm, neural crest and surface ectoderm.

By measuring expression of particular genes and/or protein markers, progress of differentiation of stem cells toward the one or more cell lineage may be detected and their progression monitored. Methods for measuring and assessing expression of genes and/or protein markers in cultured or isolated cells are those standard and known in the art. For example, such methods include quantitative reverse transcriptase polymerase chain reaction (RT-PCR), Northern blots, hybridization, and immunoassays, such as immunohistochemical analysis of sectioned material, immunostaining and fluorescence imaging, Western blotting, and for markers that are accessible in intact cells, flow cytometry analysis (FACS). In particular, isolating lineage specific cells is effected by sorting of cells via fluorescence activated cell sorter (FACS).

Various growth factors and other chemical signals may modulate differentiation of stem cells into progeny cell cultures of the one or more particular desired cell lineages. Differentiation factors that may be used in the present invention include but are not limited to compounds or molecules that modulate the activity of one or more of TGFβ/Nodal signaling, Wnt signaling, PI3K/mTOR signaling, BMP signaling, growth factor signaling, retinoic acid, FGF/MAPK or Hedgehog.

In one embodiment, the modulators of TGFβ/Nodal signaling, may include but are not limited to activators such as Activin A, TGFβ1, TGFβ2, TGFβ3, 1DE1/2 or Nodal, or may include but are not limited to inhibitors such as A-83-01 (3-(6-Methyl-2-pyridinyl)-N-phenyl-4-(4-quinolinyl)-1H-pyrazole-1-carbothioamide), SB431542 (4-[4-(1,3-benzodioxol-5-yl)-5-pyridin-2-yl-1H-imidazol-2-yl]benzamide), SB-505124 (2-[4-(1,3-Benzodioxol-5-yl)-2-(1,1-dimethylethyl)-1H-imidazol-5-yl]-6-methyl-pyridine), IDE1 (1-[2-[(2-Carboxyphenyl)methylene]hydrazide]heptanoic acid), IDE2 (Heptanedioic acid-1-(2-cyclopentylidenehydrazide), Lefty1 and Lefty 2. In one embodiment, the modulators of Wnt signaling may include but are not limited to activators such as CHIR99201 (6-[[2-[[4-(2,4-dichlorophenyl)-5-(5-methyl-1H-imidazol-2-yl)-2 pyrimidinyl]amino]ethyl]amino]-3-pyridinecarbonitrile, A1070722 (1-(7-Methoxyquinolin-4-yl)-3-[6-(trifluoromethyl)pyridin-2-yl]urea), Wnt3a, acetoxime or family members of the Wnt signaling pathway, or may include but are not limited to inhibitors such as C59 (2-(4-(2-methylpyridin-4-yl)phenyl)-N-(4-(pyridin-3-yl)phenyl)acetamide), IWP2 (N-(6-Methyl-2-benzothiazolyl)-2-[(3,4,6,7-tetrahydro-4-oxo-3-phenylthieno[3,2-d]pyrimidin-2-yl)thio]-acetamide), Dkk1, XAV939 (3,5,7,8-Tetrahydro-2-[4-(trifluoromethyl)phenyl]-4H-thiopyrano[4,3-d]pyrimidin-4-one), IWR1 (4-(1,3,3a,4,7,7a-Hexahydro-1,3-dioxo-4,7-methano-2H-isoindol-2-yl)-N-8-quinolinyl-Benzamide) FH-535=(2,5-Dichloro-N-(2-methyl-4-nitrophenyl)benzenesulfonamide.

iCRT-14=5-[[2,5-Dimethyl-1-(3-pyridinyl)-1H-pyrrol-3-yl]methylene]-3-phenyl-2,4-thiazolidinedion), JW-55 (N-[4-[[[[Tetrahydro-4-(4-methoxyphenyl)-2H-pyran-4-yl]methyl]amino]carbonyl]phenyl]-2-furancarboxamide), JW-67 (Trispiro[3H-indole-3,2′-[1,3]dioxane-2″,3′″-[3H]indole]-2,2′″(1H,1′″H)-dione) or Fzd8 (Frizzled8).

In one embodiment, the modulator of PI3K/mTOR signaling may include but are not limited to inhibitors such as PI-103 (3-[4-(4-morpholinyl)pyrido[3′,2′:4,5]furo[3,2-d]pyrimidin-2-yl]-phenol), PIK-90 (N-(2,3-dihydro-7,8-dimethoxyimidazo[1,2-c]quinazolin-5-yl)-3-pyridinecarboxamide), or LY294002 (2-(4-morpholinyl)-8-phenyl-4H-1-benzopyran-4-one), AS-252424 (5-[[5-(4-Fluoro-2-hydroxyphenyl)-2-furanyl]methylene]-2,4-thiazolidinedione), AS-605240 (5-(6-Quinoxalinylmethylene)-2,4-thiazolidine-2,4-dione), AZD-6482 ((−)-2-[[(1R)-1-[7-Methyl-2-(4-morpholinyl)-4-oxo-4H-pyrido[1,2-a]pyrimidin-9-yl]ethyl]amino]benzoic acid), BAG-956 (α,α,-Dimethyl-4-[2-methyl-8-[2-(3-pyridinyl)ethynyl]-1H-imidazo[4,5-c]quinolin-1-yl]-benzeneacetonitrile), CZC-24832 (5-(2-Amino-8-fluoro[1,2,4]triazolo[1,5-a]pyridin-6-yl)-N-(1,1-dimethylethyl)-3-pyridinesulfonamide), GSK-1059615 (5-[[4-(4-Pyridinyl)-6-quinolinyl]methylene]-2,4-thiazolidenedione), Compound 401 (2-(4-Morpholinyl)-4H-pyrimido[2,1-a]isoquinolin-4-one), PF-05212384 (N-[4-[[4-(Dimethylamino)-1-piperidinyl]carbonyl]phenyl]-N′-[4-(4,6-di-4-morpholinyl-1,3,5-triazin-2-yl)phenyl]urea).

In one embodiment, the modulators of BMP signaling may include but are not limited to inhibitors such as DM3189/LDN-193189 (4-(6-(4-(piperazin-1-yl)phenyl)pyrazolo[1,5-a]pyrimidin-3-yl)quinoline hydrochloride), noggin, chordin, or dorsomorphin (6-[4-(2-piperidin-1-ylethoxy)phenyl]-3-pyridin-4-ylpyrazolo[1,5-a]pyrimidine), or DMH1 (4-(6-(4-Isopropoxyphenyl)pyrazolo[1,5-a]pyrimidin-3-yl)quinolone) or may include but are not limited to activators such as Bmp4 and Bmp2.

In one embodiment, the modulators of FGF/MAPK may include but are not limited to inhibitors such as PD0325901 (N-[(2R)-2,3-Dihydroxypropoxy]-3,4-difluoro-2-[(2-fluoro-4-iodophenyl)amino]-benzamide), PD173074 (N-[2-[[4-(Diethylamino)butyl]amino]-6-(3,5-dimethoxyphenyl)pyrido[2,3-d]pyrimidin-7-yl]-N′-(1,1-dimethylethyl)urea), PD-161570 (N-[6-(2,6-Dichlorophenyl)-2-[[4-(di ethylamino)butyl]amino]pyrido[2,3-d]pyrimidin-7-yl]-N′-(1,1-dimethylethyl)urea) or FIIN 1 hydrochloride (N-(3-((3-(2,6-dichloro-3,5-dimethox yphenyl)-7-(4-(diethylamino)butylamino)-2-oxo-3,4-dihydropyrimido[4,5-d]pyrimidin-1(2H)-yl)methyl)phenyl)acrylamide), FR-180204 (5-(2-Phenyl-pyrazolo[1,5-a]pyridin-3-yl)-1H-pyrazolo[3,4-c]pyridazin-3-ylamine) and SU5402 (2-[(1,2-Dihydro-2-oxo-3H-indol-3-ylidene)methyl]-4-methyl-1H-pyrrole-3-propanoic acid). In one embodiment, the modulators of Hedgehog may include but are not limited to inhibitors such as SANT1 (N-[(3,5-dimethyl-1-phenyl-1H-pyrazol-4-yl)methylene]-4-(phenylmethyl)-1-piperazinamine), cyclopamine or derivatives thereof, vismodegib, IPI-926 (N-((2S,3R,3aS,3′R,4a′R,6S,6a′R,6b'S,7aR,12a'S,12b'S)-3,6,11′,12b′-tetramethyl-2′,3a,3′,4,4′,4a′,5,5′,6,6′,6a′,6b′,7,7a,7′,8′,10′,12′,12a′,12 b′-icosahydro-1′H,3H-spiro[furo[3,2-b]pyridine-2,9′-naphtho[2,1-a]azulen]-3′-yl)methanesulfonamide), LDE225 (N-(6-((2R,6S)-2,6-dimethylmorpholino)pyridin-3-yl)-2-methyl-4′-(trifluoromethoxy)-[1,1′-biphenyl]-3-carboxamide), XL139 (N-(2-methyl-5-((methylamino)methyl)phenyl)-4-((4-phenylquinazolin-2-yl)amino)benzamide) and PF-0449913.

In one embodiment, the modulator of growth factor signaling may include but are not limited to family member proteins of any one of the signaling pathways of Adrenomedullin (AM), Angiopoietin (Ang), Autocrine motility factor, Bone morphogenetic proteins (BMPs), Brain-derived neurotrophic factor (BDNF), Epidermal growth factor (EGF), Erythropoietin (EPO), Fibroblast growth factor (FGF), Glial cell line-derived neurotrophic factor (GDNF), Granulocyte colony-stimulating factor (G-CSF), Granulocyte macrophage colony-stimulating factor (GM-CSF), Growth differentiation factor-9 (GDF9), Hepatocyte growth factor (HGF), Hepatoma-derived growth factor (HDGF), Insulin-like growth factor (IGF), Migration-stimulating factor, Myostatin (GDF-8), Nerve growth factor (NGF) and other neurotrophins, Platelet-derived growth factor (PDGF), Thrombopoietin (TPO), Transforming growth factor alpha(TGF-α), Tumor necrosis factor-alpha(TNF-α), Vascular endothelial growth factor (VEGF), placental growth factor (PlGF), Foetal Bovine Somatotrophin (FBS), IL-1, IL-2, IL-3, IL-4, IL-5, IL-6 or IL-7. In one embodiment, the modulator of growth factor signaling may include but is not limited to a FGF signaling ligand such as any one of the family of FGF proteins.

In one embodiment, the modulators of retinoic acid may include but are not limited to activators such as retinoic acid precursors, All-trans retinoic acid or vitamin A. The activators of All-trans retinoic acid (ATRA) may include but are not limited to 3,7-Dimethyl-9-(2,6,6-trimethyl-1-cyclohexen-1-yl)-2E,4E,6E,8E,-nonatetraenoic acid; alternative embodiments of ATRA are 9-cis retinoic acid and 13-cis retinoic acid (IUPAC name of 9-cis retinoic acid is 3,7-Dimethyl-9-(2,6,6-trimethyl-1-cyclohexen-1-yl)nona-2E,4E,6Z,8E-tetraenoic acid and 13-cis retinoic acid is (2Z,4E,6E,8E)-3,7-dimethyl-9-(2,6,6-trimethylcyclohexen-1-yl)nona-2,4,6,8-tetraenoic acid.)

The activators of TGFβ/Nodal signaling, BMP signaling or growth factor signaling, may be used in an amount from about 0.01 ng/ml to about 20 μg/ml, or from about 0.5 ng/ml to about 15 μg/ml, or about 1 ng/ml to about 10 μg/ml, or about ng/ml to about 10 μg/ml, or about 15 ng/ml to about 5 μg/ml. The inhibitors of TGFβ/Nodal signaling, activator of Wnt signaling, inhibitors of PI3K/mTOR signaling, inhibitors of BMP signaling, activators of retinoic acid, inhibitor of hedgehog may be used in an amount that ranges from about 0.1 nM to about 200 mM, or from about 0.5 nM to about 150 mM, or about 0.5 nM to about 100 mM, or about 1 nM to about 100 mM.

Accordingly, the present invention provides for a method of differentiating stem cells into one or more cell lineages comprising contacting said cells with one or more activators of TGFβ/Nodal signaling, and one or more activators of Wnt signaling.

In one embodiment the one or more cell lineage is of the anterior primitive streak cell lineage.

In one embodiment, the one or more modulators of TGFβ/Nodal signaling may be selected from Activin A, TGF-β1, TGF-β2 or nodal. In one embodiment the one or more activators of Wnt signaling may be selected from CHIR99201, Wnt3a or other family members of the Wnt signaling pathway.

In another embodiment, the stem cells are further contacted with one or more inhibitors of PI3K/mTOR signaling. In one embodiment, the one or more inhibitors of PI3K/mTOR signaling may be selected from PI-103, PIK-90 or LY294002.

In one embodiment the cells may be contacted with Activin A, PI-103 and CHIR99201.

In another embodiment, the stem cells may be contacted with Activin A in an amount from about 1 ng/ml to 10 μg/ml, and with CHIR99201 in an amount from about 1 nM to 100 mM.

In another embodiment, the stem cells are contacted with Activin A in an amount of about 100 ng/ml, PI-103 in an amount of about 50 nM and CHIR99201 in an amount of about 2 μM.

In another embodiment, the stem cells are further contacted with one or more growth factors selected from the group consisting of Adrenomedullin (AM), Angiopoietin (Ang), Autocrine motility factor, Bone morphogenetic proteins (BMPs), Brain-derived neurotrophic factor (BDNF), Epidermal growth factor (EGF), Erythropoietin (EPO), Fibroblast growth factor (FGF), Glial cell line-derived neurotrophic factor (GDNF), Granulocyte colony-stimulating factor (G-CSF), Granulocyte macrophage colony-stimulating factor (GM-CSF), Growth differentiation factor-9 (GDF9), Hepatocyte growth factor (HGF), Hepatoma-derived growth factor (HDGF), Insulin-like growth factor (IGF), Migration-stimulating factor, Myostatin (GDF-8), Nerve growth factor (NGF) and other neurotrophins, Platelet-derived growth factor (PDGF), Thrombopoietin (TPO), Transforming growth factor alpha(TGF-α), Tumor necrosis factor-alpha(TNF-α), Vascular endothelial growth factor (VEGF), placental growth factor (PlGF), Foetal Bovine Somatotrophin (FBS), IL-1, IL-2, IL-3, IL-4, IL-5, IL-6 or IL-7.

In one embodiment, the modulator of growth factor signaling may include but is not limited to a FGF signaling ligand such as any one of the family of FGP proteins. In one embodiment, the FGF family member protein may be in an amount from about 1 ng/ml to about 1000 ng/ml. In one embodiment, the FCP family member protein may be in an amount from about 15 ng/ml to about 40 ng/ml. In another embodiment, the FGF family member protein is FGF2 in an amount of 20 ng/ml.

In a further embodiment, the anterior primitive streak cell lineage may have elevated gene or protein expression of anterior streak or pan-streak markers, including but not limited to BRACHYURY, FOXA2, CSC, FZD8, HHEX, LHX1 and/or EOMES and decreased expression of posterior streak markers including but not limited to MESP1 and EVX1 relative to undifferentiated cells.

In one embodiment, the differentiation of the stem cells in to the one or more cell lineages will be completed from about 12 to 64 hours, 12 to 72 hours, 18 to 72 hours, 18 to 66 hours, 18 to 60 hours or 24 to 60 hours. In one embodiment, the differentiation of the stem cells in to the one or more cell lineages may be completed from about 24 to 60 hours.

In one embodiment, the differentiation of the stem cells in to the cells of anterior primitive streak (APS) lineage may be completed within a period from about 12 to 84 hours, 12 to 72 hours, le to 72 hours, 18 to 66 hours, 18 to 60 hours or 24 to 60 hours. In one embodiment, the differentiation of the stem cells to APS may be completed from about 24 to 27 hours.

In the event that the stem cells have been differentiated into an anterior primitive streak cell lineage, the anterior primitive streak cells may be further differentiated into cells of the definitive endoderm (DE) lineage. Accordingly, in another embodiment the cells of the anterior primitive streak lineage obtained by the method disclosed herein are further differentiated into cells of definitive endoderm (DE) lineage, by contacting said cells of the anterior primitive streak lineage with one or more activators of TGFβ/Nodal signaling, one or more inhibitors of BMP signaling and one or more inhibitors of WNT signaling.

In one embodiment, the one or more modulators of TGFβ/Nodal signaling may be selected from may be selected from Activin A, TGF-β1, TGF-β2 or nodal.

In one embodiment, the one or more inhibitors of BMP signaling may be selected from DM3189/LDN-193189, noggin, chordin, dorsomorphin or DMH1.

In another embodiment, the anterior primitive streak cells are contacted with Activin A in an amount from about 1 ng/ml to 10 μg/ml, and with LDN-193189 in an amount from about 1 nM to 100 mM.

In another embodiment, the stem cells are further contacted with one or more inhibitor of PI3K/mTOR signaling in an amount of about 1 nM to 10 mM. In another embodiment, the anterior primitive streak cells are contacted with Activin A in an amount of about 100 ng/ml and LDN-193189 in an amount of about 250 nM.

In one embodiment, the cells of the defined endoderm lineage may have elevated gene or protein expression of endoderm markers including but not limited to FOXA2, HHEX, FZD8, CER1, SOX17 and FOXA1 and decreased pluripotency gene or protein expression including but not limited to SOX2, NANOG and OCT4 relative to undifferentiated cells.

In another embodiment, the cells of the defined endoderm lineage comprise a decreased gene or protein expression of mesoderm markers including but not limited to MESP1, MESP2, FOXF1, BRACHYURY, HAND1, EVX1, IRX3, CDX2, TBX6, MIXL1, ISL1, SNAI2, FOXC1 and PDGFRα.

In one embodiment, the differentiation of the anterior primitive streak cells in to the cells of definitive endoderm (DE) lineage may be completed within a period from about 12 to 120 hours, 12 to 114 hours, 18 to 114 hours, 18 to 108 hours, 24 to 108 hours, 24 to 102 hours or 24 to 96 hours. In one embodiment, the differentiation of the anterior primitive streak cells in to the cells of definitive endoderm (DE) lineage may be completed within a period from about 24 to 96 hours.

In one embodiment, the cells of the definitive endoderm (DE) lineage obtained by the methods described herein may be further differentiated into cells of any one of the anterior foregut (AFG), posterior foregut (PFG) or the midgut/hindgut (MHG).

Accordingly, in one embodiment the cells of the definitive endoderm (DE) lineage may be further differentiated into cells of the anterior foregut (AFG) by contacting said DE cells with a TGFβ inhibitor and a BMP inhibitor.

In one embodiment, the TGFβ inhibitor may be selected from A-83-01, SB431542, Lefty1 or Lefty2. In one embodiment, the BMP inhibitor may be selected from DM3189/LDN-193189, noggin, chordin, or dorsomorphin.

In another embodiment, the definitive endoderm cells are contacted with A-83-01 in an amount from about 1 nM to 100 mM, and with LDN-193189 in an amount from about 1 nM to 100 mM. In another embodiment, the definitive endoderm cells are contacted with A-83-01 in an amount of about 1 μM and LDN-193189 in an amount of about 250 nM.

In one embodiment, the cells of the anterior foregut comprise elevated gene or protein expression levels of anterior foregut markers including but not limited to OTX2, IRX3, TBX1, PAX9, SOX2 without either posterior foregut (PFG) or midgut/hindgut (MHG) transcription factors and relative to undifferentiated cells.

In one embodiment, the cells of the definitive endoderm (DE) lineage may be further differentiated into cells of the posterior foregut (PFG) by contacting said cells of the DE with retinoic acid, a BMP inhibitor, a Wnt inhibitor and a FGF/MAPK inhibitor.

In another embodiment, the BMP inhibitor comprises LDN193189, Wnt inhibitor comprises IWP2 and the FGF/MAPK inhibitor comprises PD0325901.

In another embodiment, the definitive endoderm cells are contacted with about 1 nM to 100 mM retinoic acid, about 1 nM to 100 mM of LDN193189, about 1 nM to 100 mM of IWP2 and about 1 nM to 100 mM of PD0325901. In another embodiment, the definitive endoderm cells are contacted with about 2 μM retinoic acid, about 250 nM of LDN193189, about 4 μM of IWP2 and about 0.5 μM of PD0325901.

In one embodiment, the cells of the posterior foregut may have elevated gene or protein expression levels of posterior foregut gene or protein expression including but not limited to the expression of SOX2, ODD1, PDX1, HNF1β, HNF4α, HNF6, and HOXA1 without either MHG or AFG gene or protein expression and relative to undifferentiated cells.

In one embodiment, the cells of the definitive endoderm (DE) lineage may be further differentiated into cells of the midgut/hindgut (MHG), by contacting said DE cells with a BMP activator, a Wnt activator and FGF activator.

In one embodiment, the BMP inhibitor comprises BMP4, the FGF activator comprises FGF2 and the Wnt activator comprises CHIR99201.

In another embodiment, the definitive endoderm cells are contacted with about 1 ng/ml to 10 μg/ml of BMP4, about 1 ng/ml to 10 μg/ml of FGF2, and about 1 nM to 10 μM of CHIR99201. In another embodiment, the definitive endoderm cells are contacted with about 10 ng/ml of BMP4, about 100 ng/ml of FGF2, and about 3 μM of CHIR99201.

In one embodiment, the cells of the posterior foregut may have elevated gene or protein expression levels of MHG markers including but not limited to CDX2, EVX1, and 5′HOX cluster genes relative to undifferentiated cells.

In one embodiment, the differentiation of the definitive endoderm cells in to any one of any one of the anterior foregut (AFG), posterior foregut (PFG) or the midgut/hindgut (MHG) may be completed within a period from about 12 to 300 hours, 12 to 280 hours, 18 to 280 hours, 18 to 260 hours, 24 to 260 hours, 24 to 250 hours or 24 to 24d hours. In one embodiment, the differentiation of the definitive endoderm cells in to any one of any one of the anterior foregut (AFG), posterior foregut (PFG) or the midgut/hindgut (MHG) will be completed within a period from 24 to 240 hours.

In another embodiment, the cells of the posterior foregut (PFG) may be induced from the definitive endoderm within three days to further differentiate into pancreatic progenitor cells by contacting said PFG with one or more FGF/MAPK inhibitors; one or more Hedgehog inhibitors; one or more BMP inhibitors; one or more Wnt inhibitors; retinoic acid (RA), and Activin A.

In one embodiment, the FGF/MAPK inhibitor comprises PD0325901 or PD173074, the hedgehog inhibitor comprises SANT 1, the BMP inhibitor comprises LDN193189, and the Wnt activator comprises IWP2 or C59.

In another embodiment, the PFG cells may be contacted with about 1 nM to 100 mM of PD0325901 or PD173074, about 1 nM to 100 mM of SALT 1, about 1 nM to 100 mM of LDN193189, about 1 nM to 100 mM of IWP2 or C59, about 1 nM to 100 mM of retinoic acid and about 1 ng/ml to 10 μg/ml of Activin A. In another embodiment, the PFG cells may be contacted with about 0.5 μM of PD0325901 or 100 nM of PD173074, about 150 nM of SANT 1, about 250 nM of LDN193189, about 4 μM of IWP2, about 2 μM of retinoic acid and about 10 ng/ml of Activin A.

In another embodiment, the cells of the pancreatic progenitors may have elevated gene or protein expression levels of pancreatic genes including but not limited to PDX1 genes relative to undifferentiated cells, and exclude hepatic progenitor gene or protein expression including but not limited to AFP and HNF4A. In another embodiment, the cells of the pancreatic progenitors comprise elevated expression levels of pancreatic genes including but not limited to PDX1 genes relative to undifferentiated cells and exclude hepatic progenitor gene or protein expression including but not limited to AFP and HNF4A.

In one embodiment, the cells of the posterior foregut (PFG) may be induced from the definitive endoderm within four days to further differentiate into liver progenitor cells by contacting said PFG with: one or more TGFβ inhibitors; retinoic acid (RA); one or more BMP activators, and one or more Wnt inhibitors.

In one embodiment, the TGFβ inhibitors comprise A83-01, the one or more BMP activators comprise BMP4, and the one or more Wnt inhibitors comprise IWP2 or C59.

In another embodiment, the PFG is contacted with about 1 nM to 100 mM of A83-01, about 1 nM to 100 mM of RA, about 1 ng/ml to 10 μg/ml of BMP4, and about 1 nM to 100 mM of IWP2 or C59. In another embodiment, the PFG is contacted with about 1 μM of A83-01, about 2 μM of RA, about 10 ng/ml of BMP4, and about 4μM of IWP2.

In another embodiment, the cells of the liver progenitors may comprise elevated gene or protein expression levels of hepatic genes including but not limited to AFP and HNF4A genes relative to undifferentiated cells and exclude pancreatic progenitor gene or protein expression including but not limited to PDX1.

In one embodiment, there is provided a method of differentiating anterior primitive streak cells into cells of definitive endoderm (DE) lineage, by contacting said cells of the anterior primitive streak lineage with one or more activators of TGFβ/Nodal signaling, and one or more inhibitors of BMP signaling, or one or more inhibitors of Wnt signaling.

In one embodiment, there is provided a method of differentiating cells of the DE into cells of the AFG, by contacting said DE cells with a TGFβ inhibitor and a BMP inhibitor.

In one embodiment, there is provided a method of differentiating cells of the DE lineage into cells of the PFG, by contacting said cells of the DE with retinoic acid, a BMP inhibitor, a Wnt inhibitor and a FGF/MAPK inhibitor.

In one embodiment, there is provided a method of differentiating cells of the DE lineage into cells of the MHG, by contacting said DE cells with a BMP activator, a Wnt activator and an FGF activator.

In one embodiment, there is provided a method of inducing pancreatic progenitors of the PFG from the DE within three days by contacting said PFG with one or more FGF/MAPK inhibitors; one or more. Hedgehog inhibitors; one or more BMP inhibitors; one or more WNT inhibitors; retinoic acid (RA), and Activin A.

In one embodiment, there is provided a method of inducing liver progenitors of the PFG from the DE within four days by contacting said PFG with one or more TGFβ inhibitors; retinoic acid, one or more BMP activators, and one or more Wnt inhibitors.

In the methods described herein, the step of contacting the cells may include culturing the cells in a suitable culture medium that is able to support the propagation and/or differentiation of cells into the intended cell lineage. In particular, the contacting of the cell is intended to include incubating the cell in a culture medium together with one or more of the differentiating factors in vitro. The term “contacting” is not intended to include the in vivo exposure of cells to differentiating factors, and may be conducted in any suitable manner. For example, the cells may be treated in adherent culture, or in suspension culture that include one or more differentiating factors. It is understood that the cells contacted with one or more differentiating factors may be further treated with other cell differentiation environments to stabilize the cells, or to differentiate the cells further.

In one embodiment, the stem cells are contacted with one or more differentiating factors in a culture medium that may be supplemented with other factors or otherwise processed to adapt it for propagating, maintaining or differentiation of the cells lineages. To maintain stem cell pluripotency, for example, the stem cells and cell lineages disclosed herein may be cultured in conditioned medium, such as mEF-CM, or fresh serum-free medium alone, mTesR, or other hPSC maintenance media that are known in the art or xeno-free media such as Essential 8. To differentiate stem cells the stem cells and cell lineages disclosed herein may be cultured in a feeder free medium or medium comprising a feeder layer, whereby the culture mediums may be chemically defined as containing Iscove's Modified Dulbecco's Media (IMDM), F12, transferrin, insulin, concentrated lipids, or polyvinyl alcohol (PVA). The pluripotency maintaining media may be used for differentiation. Alternatively, for differentiation a basal media may be used derived from minimal basal media that contain the basic ingredients for cell survival and growth known in the art, and that do not contain added growth factors/chemicals that confound differentiation.

In one embodiment, the culture medium may be a conditioned medium obtained from a feeder layer. It is contemplated that the feeder layer comprises fibroblasts, and in one embodiment, comprises embryonic fibroblasts.

In one embodiment of the present invention, a feeder cell layer is generated by a method which essentially involves culturing the cells that will form the feeder layer and inactivating the cells. The cells that will form the feeder cell layer may be cultured on a suitable culture substrate. In one embodiment, the suitable culture substrate is an extracellular matrix component, such as, for example, those derived from basement membrane or that may form part of adhesion molecule receptor-ligand couplings. In one embodiment, a suitable culture substrate is MATRIGEL® (Becton Dickenson). MATRIGEL® is a soluble preparation from Engelbreth-Holm-Swarm tumor cells that gels at room temperature to form a reconstituted basement membrane. In another embodiment, the suitable culture substrate is gelatin (Sigma). Other extracellular matrix components and component mixtures are suitable as an alternative. One other embodiment is Geltrex™ LDEV-Free hESC qualified reduced growth factor basement membrane matrix. Depending on the cell type being proliferated, this may include laminin, fibronectin, proteoglycan, vitronectin, entactin, heparan sulfate, and the like, alone or in various combinations.

An alternative culture system employs serum-free medium supplemented with growth factors capable of promoting the proliferation of embryonic stem cells. For example, a feeder-free, serum-free culture system in which stem cells are maintained in unconditioned serum replacement (SR) medium supplemented with different growth factors capable of triggering stem cell self-renewal.

In one embodiment, the culture medium may be a feeder-free culture medium that may not contain feeder cells or exogenously added conditioned medium taken from a culture of neither feeder cells nor exogenously added feeder cells in the culture. Of course, if the cells to be cultured are derived from a seed culture that contained feeder cells, the incidental co-isolation and subsequent introduction into another culture of some small proportion of those feeder cells along with the desired cells (e. g., undifferentiated primate stem cells) should not be deemed as an intentional introduction of feeder cells. In such an instance, the culture contains a de minimus number of feeder cells. By “de minimus”, it is meant that number of feeder cells that are carried over to the instant culture conditions from previous culture conditions where the differentiable cells may have been cultured on feeder cells. Similarly, feeder cells or feeder-like cells that develop from stem cells seeded into the culture shall not be deemed to have been purposely introduced into the culture. For example, for APS, DE, AFG, PFG (4 days protocol) and MHG differentiation a feeder free culture medium may be employed that is chemically defined and may contain PVA, insulin, transferrin, concentrated lipids, mono-thioglycerol, IMDM, or F12. Alternatively, for PFG, pancreatic and hepatic differentiation a feeder free culture medium may be employed that is chemically defined and may contain PVA, concentrated lipids, knockout serum replacement (KOSR), IMDM, F12.

Accordingly, in one embodiment the culture medium, used in the present methods described herein for the propagation and/or differentiation of the stem cells, may be substantially free of feeder cells or layers. In addition, a feeder-free culture medium may require for the stem cells to be grown on a suitable culture substrate, including any substrate coated with extracellular matrix components (i.e., collagen, laminin, fibronectin, proteoglycan, entactin, heparan sulfate, and the like, alone or in various combinations), or MATRÏGEL™. As such, in another embodiment, the stem cells may be cultured in a culture medium that is free of a feeder cell layer, with the use of a matrix component as a culture substrate. In another embodiment, the culture medium, used in the present methods described herein for the propagation and/or differentiation of the stem cells, may be substantially free of feeder cells or layers without the use of a substrate matrix, such as a suspension culture medium.

In one embodiment there is provided a cell culture medium for differentiating a stem cell into one or more cell lineages comprising one or more of the following factors: one or more FGF/MAPK inhibitors; one or more Hedgehog inhibitors; one or more BMP inhibitors, one or more WNT inhibitors, retinoic acid, Activin A, and one or more inhibitors of PI3K/mTOR signaling.

In another embodiment, the present invention provides a cell culture medium for differentiating a stem cell into one or more cell lineages comprising one or more of the following factors: a matrix component, one or more activators of TGFβ/Nodal signaling, one or more activators of Wnt/β-catenin signaling, and one or more inhibitors of PI3K/mTOR signaling.

In another embodiment, the present invention provides a cell culture medium for differentiating a stem cell into one or more cell lineages comprising one or more of the following factors: one or more activators of TGFβ/Nodal signaling, and one or more inhibitors of BMP signaling.

In another embodiment, the present invention provides a cell culture medium for differentiating a stem cell into one or more cell lineages comprising a TGFβ inhibitor and a BMP inhibitor.

In another embodiment, the present invention provides a cell culture medium for differentiating a stem cell into one or more cell lineages comprising one or more of the following factors: retinoic acid, a BMP inhibitor, a Wnt inhibitor and a FGF/MAPK inhibitor.

In another embodiment, the present invention provides a cell culture medium for differentiating a stem cell into one or more cell lineages comprising one or more of the following factors: BMP4, a Wnt activator and an FGF activator.

In another embodiment, the present invention provides a cell culture medium for differentiating a stem cell into one or more cell lineages comprising one or more of the following factors: one or more FGF/MAPK inhibitors; one or more Hedgehog inhibitors; one or more BMP inhibitors; one or more Wnt inhibitors; retinoic acid (RA), and Activin A.

In another embodiment, the present invention provides a cell culture medium for differentiating a stem cell into one or more cell lineages comprising one or more of the following factors: one or more TGFβ inhibitors; one or more BMP activators, and one or more Wnt inhibitors.

In another embodiment, the present invention provides a cell culture medium for differentiating a stem cell into one or more cell lineages comprising one or more of the following factors: one or more inhibitors of Nodal/TGFβ; one or more inhibitors of BMP; one or more inhibitors of FGF/MAPK, and one or more inhibitors of Wnt.

In another embodiment, the differentiating factors comprising activators of TGFβ/Nodal signaling, BMP signaling or modulators of growth factor signaling, may be in the cell culture system in an amount that ranges from about 0.01 ng/ml to about 20 μg/ml, or from about 0.5 ng/ml to about 15 μg/ml, or about 1 ng/ml to about 10 μg/ml, or about 10 ng/ml to about 10 μg/ml, or about 15 ng/ml to about 5 μg/ml. In addition, the differentiating factors comprising inhibitors of TGFβ/Nodal signaling, activator of Wnt signaling, inhibitors of PI3K/mTOR signaling, inhibitors of BMP signaling, activators of retinoic acid, inhibitors of FGF/MAPK, inhibitor of hedgehog may be in the cell culture system in an amount that ranges from about 0.1 nM to about 200 mM, or from about 0.5 nM to about 150 mM, or about 0.5 nM to about 100 mM, or about 1 nM to about 100 mM.

In one embodiment, there is provided a cell produced according to any of the methods described herein.

In one embodiment, there is provided a kit for use in any one of the methods described herein, comprising one or more containers of cell culture medium as described herein, together with instructions for use.

The disclosure illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features; modification and variation of the inventions embodied therein herein disclosed may be resorted to by those skilled in the art, and that such modifications and variations are considered to be within the scope of this invention.

The disclosure has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments' are within the following claims and non-limiting examples. In addition, where features or aspects of the invention are described in terms of Markush groups, those skilled in the art will recognize that the invention is also thereby described in terms of any individual member or subgroup of members of the Markush group.

### BRIEF DESCRIPTION OF THE DRAWINGS

The invention will be better understood with reference to the detailed description when considered in conjunction with the non-limiting examples and the accompanying drawings, in which:

FIG. 1 is a schematic representation of Microarray reanalysis of genes upregulated >2-fold during AFBLy treatment of H9 hESC and GO analysis.

FIG. 2 Shows test effects of increasing FGF2 (10-40 ng/mL), Wnt3a (15-100 ng/mL), CHIR99021 (50-1000 nM) or BMP4 (3-20 ng/mL) (panels i, ii, iii and iv, respectively) and respective inhibitors (100 nM PD173074, 2 μM IWP2, 150 ng/mL Dkk1 and 250 nM DM3189) on PS formation, H1 hESC were differentiated towards PS for 24 hours with indicated base combinations of Activin (100 ng/mL), FGF2 (20 ng/mL) and 10 μM LY294002 (“AFLy” or “ALy”) in conjunction with the indicated signaling perturbations, and qPCR was performed (day 1).

FIG. 3 Shows test effects of increasing BMP, FGF or Wnt signaling (10 ng/mL BMP4, 3 μM CHIR and 5-20 ng/mL FGF2; panels i, ii and iii respectively) on DE vs. mesoderm emergence from PS, H1 hESC were initially differentiated with AFBLy towards PS for 24 hours and then subsequently differentiated with AFLy, AFBLy or ALy+250 nM DM3189 (“ADLy”) for 48 subsequent hours with indicated signaling perturbations, and qPCR was performed (day 3).

FIG. 4 Shows the temporary dynamic signaling logic for primitive streak, mesoderm and definitive endoderm specification. (i) shows the necessity of BMP for MIXL1-GFP APS induction; (ii) shows BMP, FGF, Wnt and TGFβ signaling and its effect on the induced lineage; and (iii) shows a graphical representation of (ii).

FIG. 5 Shows Hl hESC differentiated by ACP for 24 hours stained for BRACHYURY, FOXA2, EOMES and LHX1 (nuclear counterstaining by DAPI), scale bar=100 μm for all subsequent figures (left); virtually all HES3 hESC are MIXL1-GFP+ after 24 hours of ACP differentiation, shown by FACS (right).

FIG. 6 Shows Microarray heatmap of independent triplicates; undifferentiated HES3 hESC (Day 0), ACP-induced APS (Day 1), SR1-induced DE (Day 3) or hESC differentiated by AFBLy or serum for 3 days.

FIG. 7 Shows FOXA2 and SOX17 staining of SR1-, serum- or AFBLy-treated H1 hESC after 3 days of differentiation (top); summary of CXCR4+PDGFRα− DE percentages in hPSC (grey) or after SR1 differentiation (blue) from 7 hPSC lines, dots represent experimental replicates (bottom left); histogram summarizing CXCR4+PDGFRα− DE percentages after differing differentiation protocols, error bars represent standard deviation (bottom right).

FIG. 8 Shows FACS analysis of H9 SOX17-mCHERRY hESC; reporter expression before or after 2 days of SR1 differentiation.

FIG. 9 Shows FACS analysis of CXCR4 and PDGFRα expression before or after SR1 differentiation from indicated hPSC lines.

FIG. 10 Shows a Single-cell qPCR heatmap of 80 H7 hESC before or after differentiation by SR1, AFBLy or serum for 2 days.

FIG. 11 Shows the neural competence of H1 hESC after 0-2 days of SR1 induction were transferred into neuralizing media (“N”, 3 days) and neural gene expression was compared to SR1-induced DE (“day 3 DE”).

FIG. 12 Shows a schematic diagram of the anterior posterior patterning of hESC-derived endoderm.

FIG. 13 Shows that transcription factors demarcrate anteroposterior domains in vivo.

FIG. 14 Shows effects of (i) increasing BMP4 (10-25 ng/mL) or (ii) increasing CHIR (3-6 μM) on MHG induction, day 3 DE was differentiated for 4 subsequent days with indicated base conditions together with designated signaling perturbations until day 7, with AFG and PFG controls indicated (subsumed by FIG. S4a); (i) FGF+CHIR=100 ng/mL FGF2+3 μM CHIR; (ii) BF=10 ng/mL BMP4+100 ng/mL FGF2.

FIG. 15 Shows OTX2, FOXA2 and CDX2 immunostaining of H1-derived day 7 AFG and MHG respectively with quantification.

FIG. 16 Shows a microarray heatmap of HES3-derived. AFG, PFG, and MHG populations on day 7 in independent triplicate.

FIG. 17 Shows qPCR of day 7 AFG, PFG, and MHG populations from H7 and HES3 hESC lines; HOX genes boxed.

FIG. 18 Shows pancreatic or hepatic competence, where day 3 DE was patterned into AFG or PFG for 1-2 days, and each was then subsequently differentiated towards pancreas or liver for 3 further days.

FIG. 19 Shows the effects of increasing amounts of (i-iii) BMP/TGFβ signaling or (iv) FGF/MAPK signaling on pancreas vs. liver induction, day 3 DE was differentiated with indicated conditions with (i-ii) 5-20 ng/mL Activin or (ii-iii) 5-10 ng/mL BMP4 and respective inhibitors (1 μM A8301, 250 nM DM3189, 100 nM PD173074, 500 nM PD0325901) where indicated. Abbreviations for base conditions: (i) RS=2 μM RA+SANT1; (ii-iii) RS+PD=RS PD0325901; (iv) DRK=DM3189 RA+KAAD-Cyclopamine.

FIG. 20 Shows depictions of (i) dynamic signaling inputs, (ii) truth table and (iii) dichotomy of BMP and TGF signaling for liver versus pancreas induction.

FIG. 21 Shows the efficient specification of Afp⁺ hepatic progenitors.

FIG. 22 Shows a substrate luciferase assay for CYP3A4 metabolic activity (i) and staining for LDLR expression and LDL-DyLight 594 uptake (ii) in hESC-derived late hepatic progeny.

FIG. 23 Shows that CAG-GFP+ hESC differentiated into early hepatic progenitors or late hepatic progeny when transplanted (top left); human albumin levels in mouse sera, each dot is an individual mouse (fractions of successfully engrafted mice indicated; top right); recipient whole-liver cross-section with different lobes and subfields indicated, scale bar=5 mm (middle right); co-staining for human albumin and GFP in four distinct hepatic lobes, fields numbered above (bottom).

FIG. 24 Shows a RNA-seq heatmap of stage-specific genes upregulated at indicated lineage transitions.

FIG. 25 Shows that APS enhancers are rapidly activated within 24 hours of differentiation.

FIG. 26 Shows that distinct active enhancer programs are invoked upon endoderm development.

FIG. 27 Shows the mutually exclusive enhancer activation in separate anteroposterior domains.

FIG. 28 Shows the top-ranked GO terms associated with DE-specific active enhancers by GREAT without pre-selection.

FIG. 29 Shows that endoderm enhancer activation correlates with nearby gene activation.

FIG. 30 Shows that endoderm-specific active enhancers are preserved.

FIG. 31 Shows a comparative list of contemporary endoderm enhancers.

FIG. 32 Shows transcription factor motifs that are enriched within endoderm-specific active enhancers.

FIG. 33 Shows joint transcription factor co-occupancy associated with endoderm enhancer activity.

FIG. 34 Shows that transcription factors and signaling effectors co-occupy active endodermal enhancers.

FIG. 35 Shows, that endodermal enhancers reside in a multiplicity of distinct “pre-enhancer” states in pluripotent cells.

FIG. 36 Shows that the frequency of endoderm pre-enhancer labeling by given chromatin factors in hESCs.

FIG. 37 Shows H2Az-only pre-enhancers attract endoderm transcription factors more readily upon differentiation.

FIG. 38 is a graphic representation of the multitude of “pre-enhancer” states in uncommitted cells.

FIG. 39 Shows the expression of both mesodermal and endodermal regulatory genes in hESC cells differentiated in AFBLy conditions.

FIG. 40 Shows that AFBLy preferentially upregulates mesoderm transcription factors.

FIG. 41 shows that the efficient endoderm formation from the primitive streak requires inhibiting endogenous BMP signaling.

FIG. 42 Shows that the late BMP blockade in the independent H1 hESC line redacts mesoderm formation.

FIG. 43 Shows that BMP inhibition expands endoderm.

FIG. 44 represents a summary of early BMP signaling dynamics.

FIG. 45 Shows that 3 independent Wnt antagonists redact mesoderm formation from the primitive streak.

FIG. 46 Shows that double BMP and Wnt inhibition is redundant to repress mesoderm formation.

FIG. 47 Shows the upregulation of endogenous signaling during differentiation.

FIG. 48 Shows that FGF is permissive for anterior and posterior PPS specification.

FIG. 49 Shows that FGF and PI-103expression levels alter from PS induction.

FIG. 50 Shows the chemical structure, specificity and efficacy of chemical PI3K inhibitors.

FIG. 51 Shows the comparative efficacy of PI3K inhibitorsLY294002, PIK-90 and PI-103 in PS differentiation.

FIG. 52 Shows the adaptation of ethnically diverse hESC lines to undifferentiated propagation in feeder-free conditions that are karotypically normal.

FIG. 53 Shows the endoderm induction on fibronectin.

FIG. 54 Shows that TGFβ and Wnt signaling and PI3K/mTOR inhibition efficiently specifies the anterior streak.

FIG. 55 Shows the FACS of HES2 and HESS differentiation by SR1.

FIG. 56 Shows the relinquishing of CD90 and Pdgfra in endoderm.

FIG. 57 Shows the gating strategy for FACS analysis.

FIG. 58 Shows that SR1 efficiently specifies definitive endoderm, absent mesoderm or other extraneous lineages from diverse hESC lines.

FIG. 59 Shows a FACS comparison between different methods.

FIG. 60 Shows the efficient induction of Sox17⁺Foxa2⁺ definitive endoderm by SR1.

FIG. 61 Shows that hESC and hiPSC are differentiated equally efficiently into endoderm by SR1.

FIG. 62 Shows the provisional signaling requirements for the anteroposterior patterning of hESC-derived definitive endoderm.

FIG. 63 Shows BMP, FGF/MAPK, Wnt and Hedgehog signaling cooperatively represses pancreatic specification.

FIG. 64 Shows the exclusion of pancreas during hepatic specification.

FIG. 65 Shows the comparison′ liver differentiation strategies from hESC.

FIG. 66 Shows the induction of albumin during hepatic maturation of hESC differentiation after a 6 day period.

FIG. 67 Shows a late, not early, hESC-derived progeny engraft.

FIG. 68 Shows the coexpression of HepPar1 and albumin by a hESC-derived engrafted progeny.

FIG. 69 Shows that engrafted hESC-derived liver cells do not express detectable levels of Afp.

FIG. 70 Shows a statistical analysis of endoderm differentiation.

FIG. 71 Shows the unilateral H3K27ac activation and accompanying PRC2 depression at CXCR4 enhancer.

FIG. 72 Shows a cell-type specific enhancer usage during anteroposterior patterning.

FIG. 73 Shows that Eomes, mad2/3, Smad 4 & Foxh1 co-occupy endoderm enhancers.

FIG. 74 Shows that other lineage enhancers are frequently inactive in SR1-induced endoderm.

FIG. 75 Shows that neural-association enhancers are active in previous hESC-derived endoderm populations.

FIG. 76 is a graphical representation of the prevalence of DE pre-enhancer classes in hESC.

FIG. 77 Shows the genomic locations of DE pre-enhancer classes.

FIG. 78 Shows the mesoderm pre-enhancer chromatin states.

FIG. 79 Shows the active enhancers that are exclusive to the anterior foregut.

FIG. 80 Shows the chromatin structures of the Hoxa locus during anterioposterior patterning.

### EXPERIMENTAL SECTION

### Materials and Methods

Undifferentiated Propagation of hESCs and hiPSCs

1. Initial Adaption from MEF Co-Culture to Defined Culture Conditions

Most hESC lines employed in this study were originally cultured on irradiated mouse embryonic fibroblast (MEF) feeder layers. To adapt hESC lines to feeder-free culture, MEF-grown hESCs were serially passaged onto Matrigel-coated plates and propagated in MEF-conditioned medium (CM).

To generate MEF-CM, confluent MEF cultures were treated with KOSR medium (DMEM/F12 supplemented with 20% KOSR (Gibco, v/v), L-glutamine, non-essential amino acids (NEAA), α-mercaptoethanol, penicillin, streptomycin and 4 ng/mL FGF2 (to stimulate MEF cytokine production)) and after 24 hours, conditioned KOSR medium was retrieved, filtered, and supplemented with additional 15 ng/mL FGF2 before being added to hESC cultures.

2. Long-Term Undifferentiated Propagation in Defined Conditions (mTeSR1)

Once hESC lines were adapted to MEF-CM culture conditions, they were then adapted to growth in mTeSR1 (StemCell Technologies). To effectuate this, two days after hESCs were plated in MEF-CM, they were transferred into mTeSR1. An initial slight impediment in hESC growth was noted upon initial transfer from MEF-CM into mTeSR1 and generally, some differentiation resulted as well. hESCs were serially passaged in mTeSR1 and overtly differentiated cells were mechanically scraped until finally undifferentiated hESCs could be stably propagated in mTeSR1 (FIG. 52). Only after hESCs were adapted to mTeSR1 in high quality (that is, spontaneous differentiation was fully eliminated) were they subsequently used for differentiation experiments. This was conducted to prevent exposure of hESCs to animal feeders or undefined media components in the undifferentiated state from confounding downstream differentiation. Eventually, the H1, H7, H9, HES2 and HES3 hESC lines were finally adapted to undifferentiated propagation in mTeSR1 and were karyotypically normal (FIG. 52).

hiPSC lines BJC1 and BJC3 were derived by transfecting the human BJ foreskin fibroblast line with mRNAs encoding the obligatory reprogramming factors (J Durruthy-Durruthy, V Sebastiano, unpublished work) and they were subsequently propagated in an undifferentiated state with mTeSR1 by techniques identical to those used to propagate and passage hESC (FIG. 52).

### Coating Cell Culture Plastics for Differentiation

Cell culture plastics were pre-coated with either human fibronectin (Millipore, FC010) or Matrigel (BD Biosciences) before plating hPSC atop for SR1 differentiation. For a single well in a 12-well plate, a well was briefly wetted with 100 μL sterile PBS to cover the entire surface area of the well, and then excess PBS was removed. Then, 200 μL of human fibronectin (diluted to 10 μg/mL in PBS) was added to the well, and left to adsorb to the surface of the well for 1 hour at 37° C. After fibronectin coating was complete, all fibronectin solution was removed and hPSC were subsequently plated. For Matrigel coating, Matrigel was first diluted 1:15 in DMEM/F12 (Gibco). Wells were briefly coated with sufficient diluted Matrigel to cover the entire surface area, and then subsequently, Matrigel was retrieved and saved for future use. Plates were then left to incubate for 15 minutes at 37° C. to enable Matrigel layer assembly. This was repeated a second time—the well was briefly covered with diluted Matrigel a second time and then left to incubate for 15 minutes at 37° C. Afterwards, residual Matrigel was aspirated and hPSC were subsequently plated.

### Defined Definitive Endoderm Specification in SR1

All hESC and hiPSC lines were propagated feeder-free in mTeSR1 (FIG. 52). Differentiation was conducted feeder-free in fully-defined, serum-free CDM2 basal medium. Prefacing differentiation, confluent hPSC cultures were passaged as small clumps with collagenase IV (typically 1:3 split ratio) onto new plates coated with either human fibronectin or Matrigel. After 1-2 days of recovery in mTeSR1, hPSCs were washed with F12 (Gibco) to evacuate all mTeSR1 and then were treated for 24 hours with Activin A (100 ng/mL, R&D Systems), CHIR99021 (2 μM, Stemgent), and PI-103 (50 nM, Tocris) in CDM2 to specify APS. Afterwards, cells were washed (F12), then treated for 48 hours with Activin A (100 ng/mL) and LDN-193189/DM3189 (250 nM, Stemgent) in CDM2 to generate DE by day 3. Media was refreshed every 24 hours.

DE was anteroposteriorly patterned into either AFG (A-83-01, 1 μM and DM3189, 250 nM), PFG (RA, 2 μM and DM3189, 250 nM), or MHG (BMP4, 10 ng/mL; CHIR99021, 3 μM; and FGF2, 100 ng/mL) for 4 subsequent days until day 7.

### Defined Anterioposterior Patterning of Definitive Endoderm in SR1

Day 3 DE was patterned into AFG, PFG, or MHG by 4 days of continued′ differentiation in CDM2. DE was washed (F12), then differentiated′ as follows: AFG, A-83-01 (1 μM, Tocris) and DM3189 (250 nM); PFG, RA (2 μM, Sigma) and DM3189 (250 nM); MHG, BMP4 (10 ng/mL, R&D Systems), CHIR99021 (3 μM), and FGF2 (100 ng/mL), yielding day 7 anteroposterior domains.

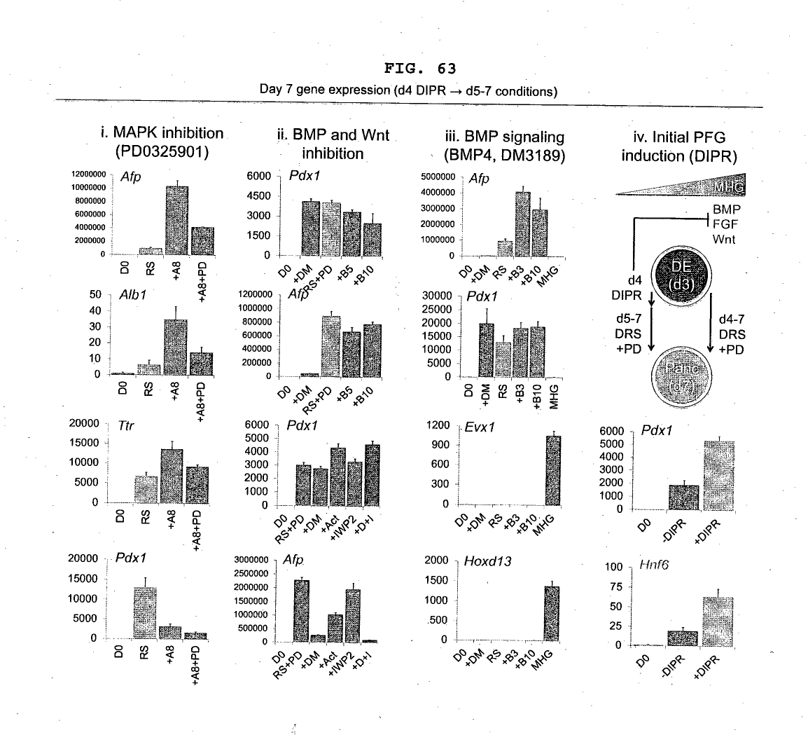

To derive hESC-derived hepatic progenitors (in CDM2+KnockOut Serum Replacement (KOSR, 10% v/v, Gibco)), day 3 DE was washed, treated with DM3189 (250 nM), IWP2 (4 μM, Stemgent), PD0325901 (500 nM, Tocris), and RA (2 μM) for 1 day towards early PFG (altogether known as “DIPR”; day 4). Subsequently, cells were washed (F12) and then differentiated 3 further days with A-83-01 (1 μM), BMP4 (10 ng/mL), IWP2 (4 μM), and RA (2 μM) to yield hepatic progenitor-containing populations on day 7 of differentiation. As detailed in FIG. 25, the rationale for a transient 1 day of DIPR treatment from DE was to use (i) RA to regionalize the PFG domain (Stafford and Prince, 2002) in conjunction with (ii) inhibition of BMP, FGF/MAPK and Wnt signaling (with DM3189, PD0325901 and IWP2, respectively) to suppress MHG formation and prevent excess posteriorization. As shown in FIG. 63iv, an initial day of DIPR treatment to provisionally specify PFG enhances subsequent pancreatic emergence.

### Preparation of CDM2 Basal Differentiation Medium

CDM2 comprising 50% IMDM (Gibco) and 50% F12 (Gibco), supplemented with 1 mg/mL polyvinyl alcohol (Sigma, A1470 or Europa Bioproducts, EQBAC62), 1% v/v chemically-defined lipid concentrate (Gibco, 11905-031), 450 DM monothioglycerol (Sigma, M6145), 0.7 μg/mL insulin (Roche, 1376497) and 15 μg/mL transferrin (Roche, 652202) was sterilely filtered (22 μm filter, Millipore) and used for differentiation within 2 weeks' time. Chemical compounds and recombinant growth factors were added to elicit different steps of differentiation as described above. hESC differentiation to APS, DE, AFG, PFG, and MHG was conducted in CDM2 alone. For hESC differentiation to early PFG (DIPR) as well as subsequent liver progenitor differentiation, CDM2 supplemented with 10% v/v KOSR was used to aid cell survival.

### Differentiation to Alternative Lineages

hESC differentiation towards DE using AFBLy (Touboul et al., 2010) or serum (D'Amour et al., 2005) was executed as previously described. For AFBLy, hESCs were briefly washed (F12) and then concomitantly treated with Activin A (100 ng/mL), FGF2 (20 ng/mL), BMP4 (10 ng/mL), and LY294002 (10 μM) for 3 consecutive days. For serum differentiation, hESCs were briefly washed (F12) and then were persistently treated with Activin A (100 ng/mL) for 3 consecutive days, combined with increasing amounts of FBS (Hyclone)-0% (day 1), 0.2% (day 2), and 2% (day 3) v/v, respectively. For purposes of direct comparison to SR1, differentiation in either AFBLy or serum was conducted in CDM2 basal medium.

### Fate Inter-Conversion Differentiation Experiments

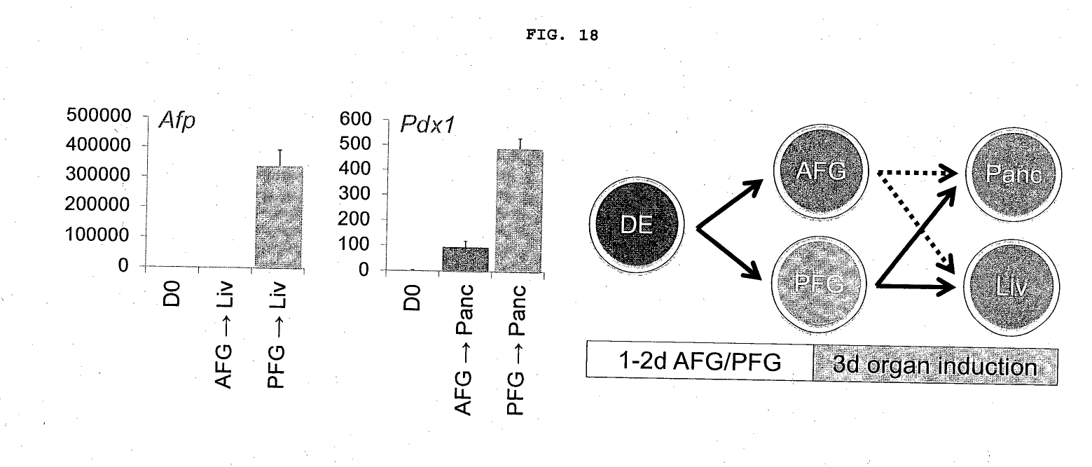

For foregut competency experiments (FIG. 18), Day 3 DE was washed (F12), transiently differentiated into AFG (A-83-01+DM3189) or PFG (RA+DM3189) for 1-2 days, and then washed again (F12) and subsequently differentiated to either pancreas or liver lineages for 3 further days (as described above).

### RNA Extraction, Reverse Transcription, and Quantitative PCR

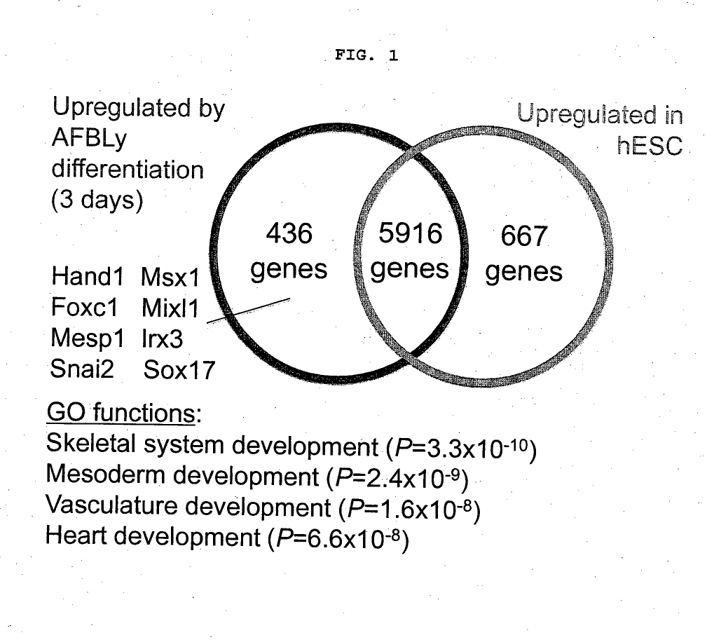

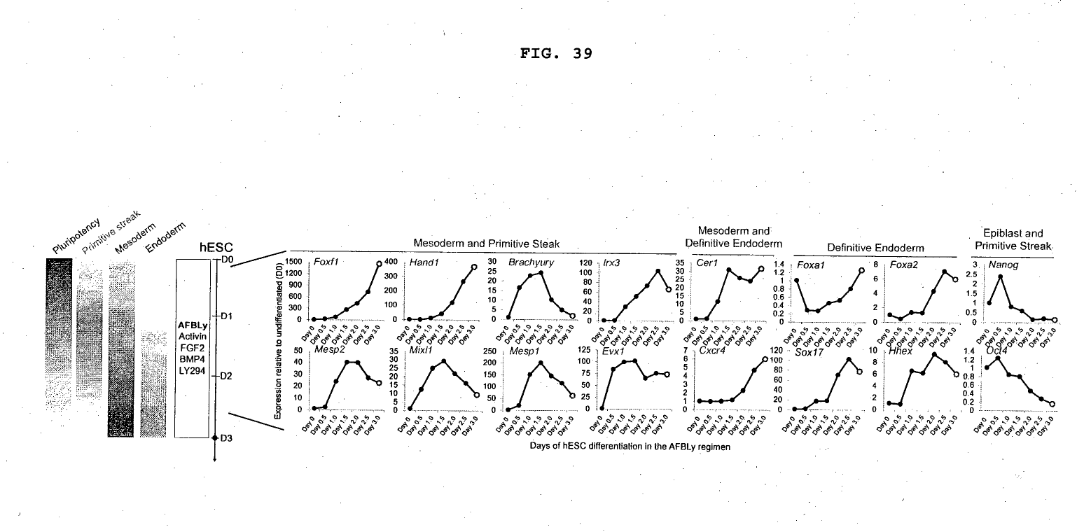

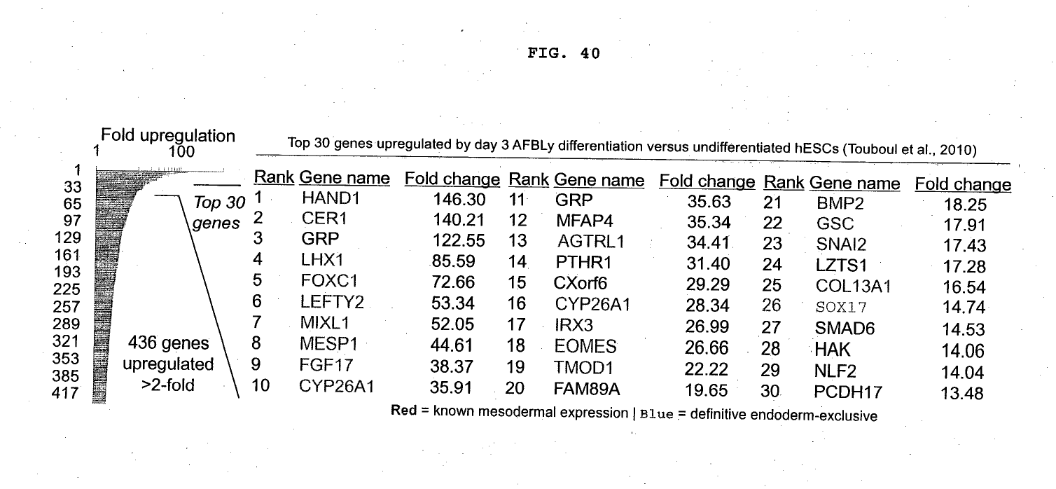

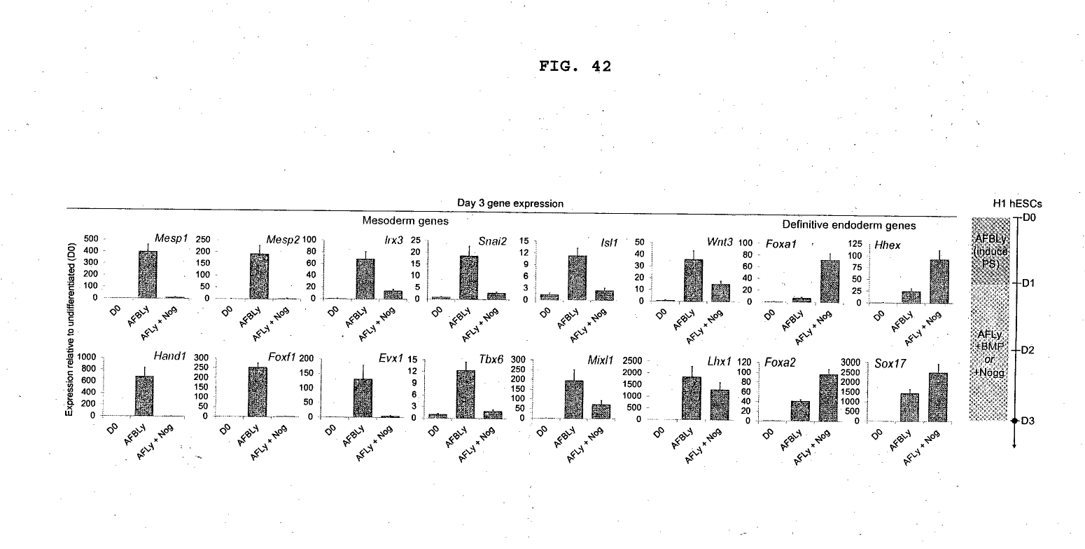

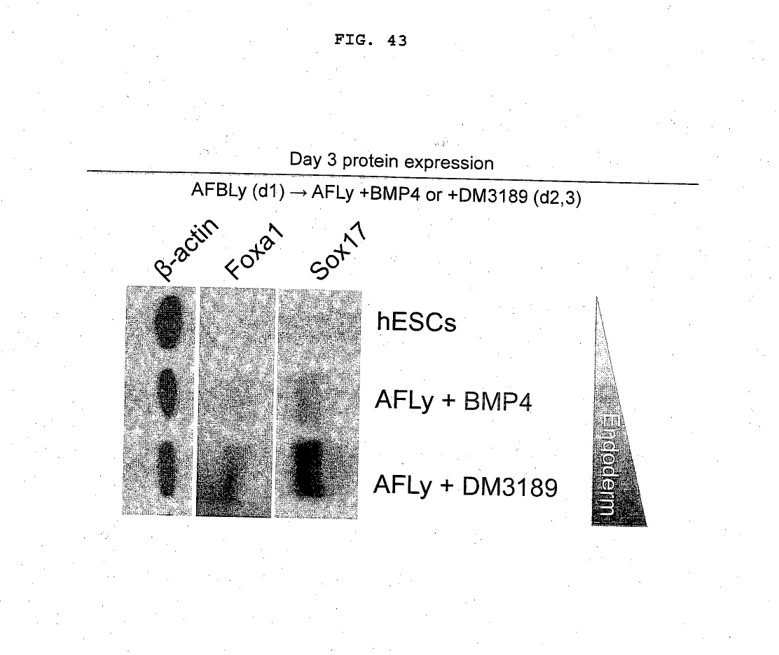

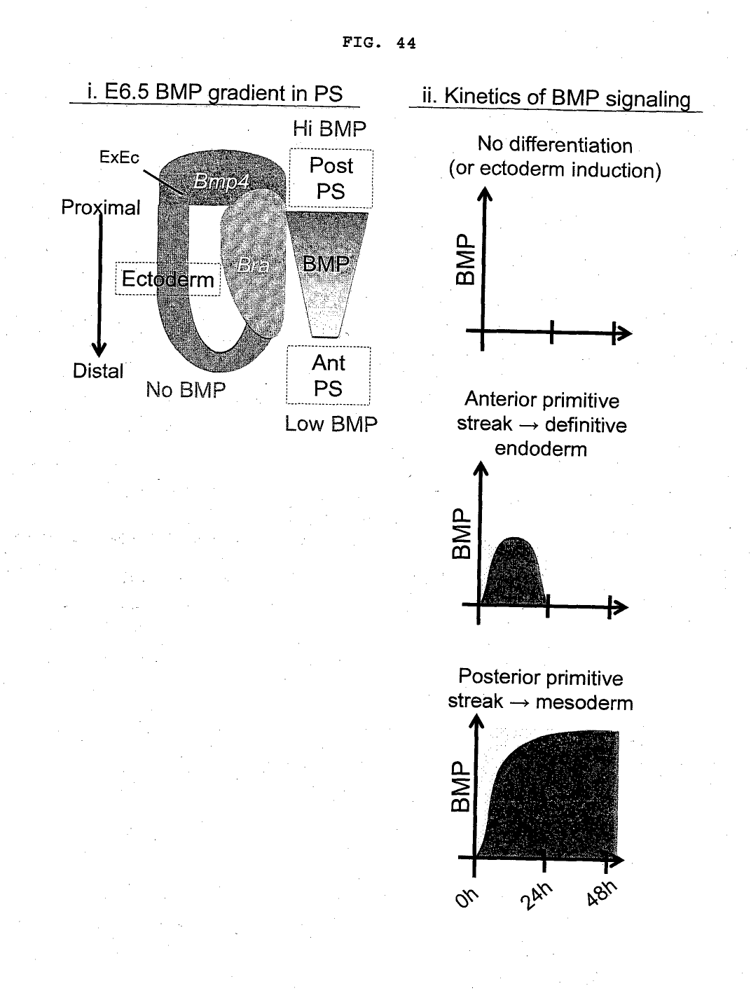

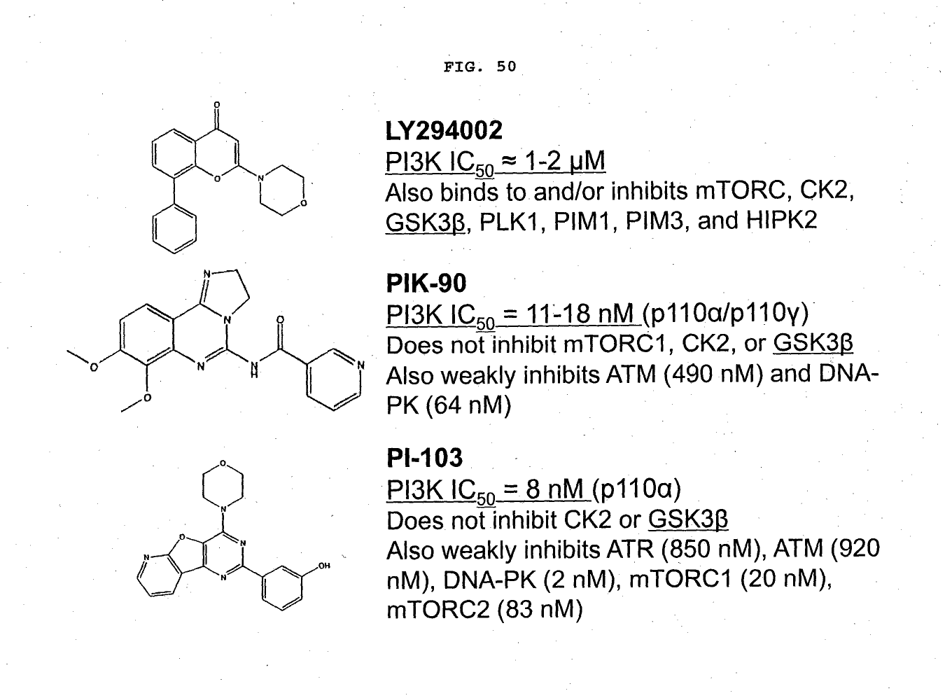

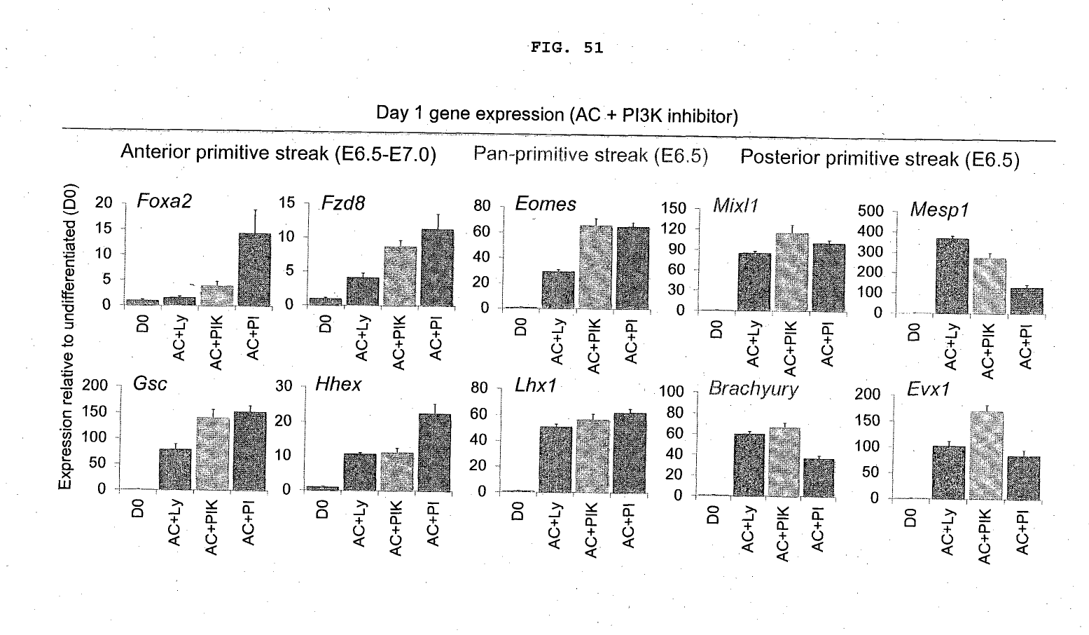

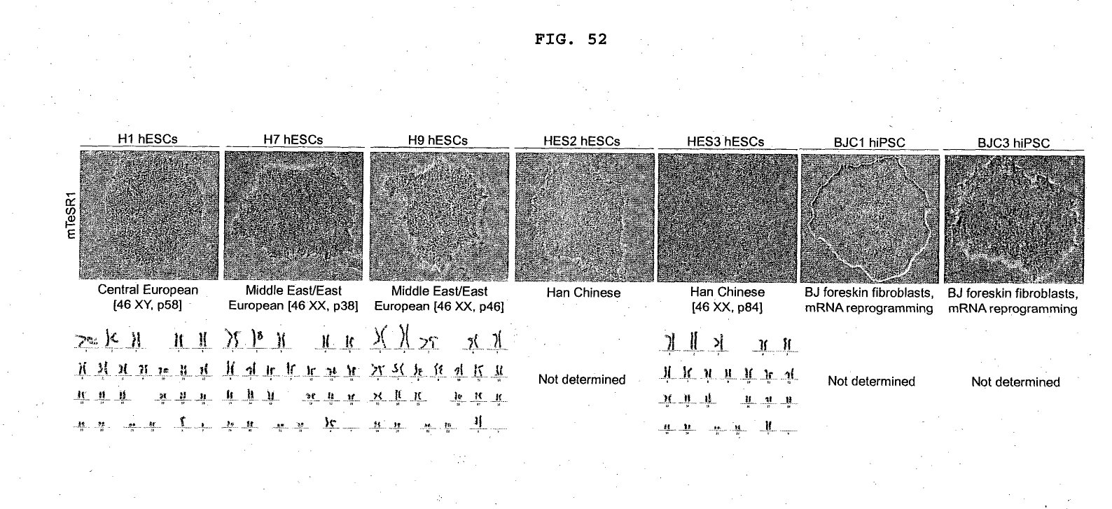

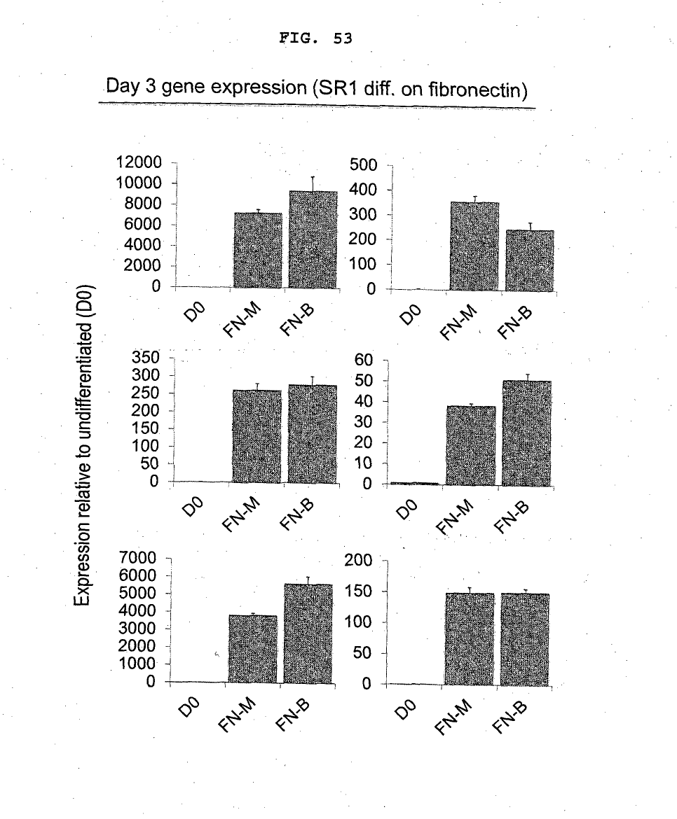

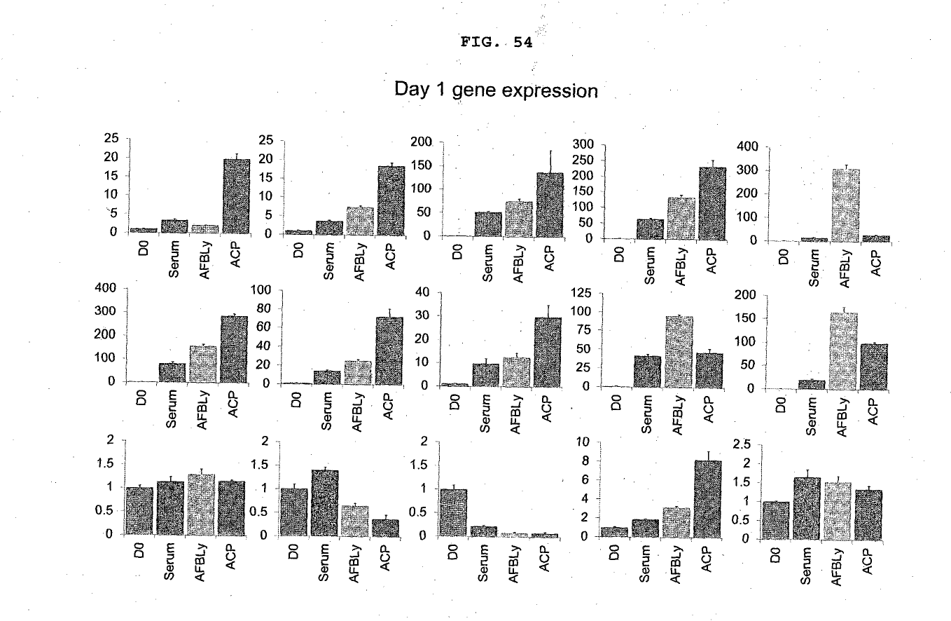

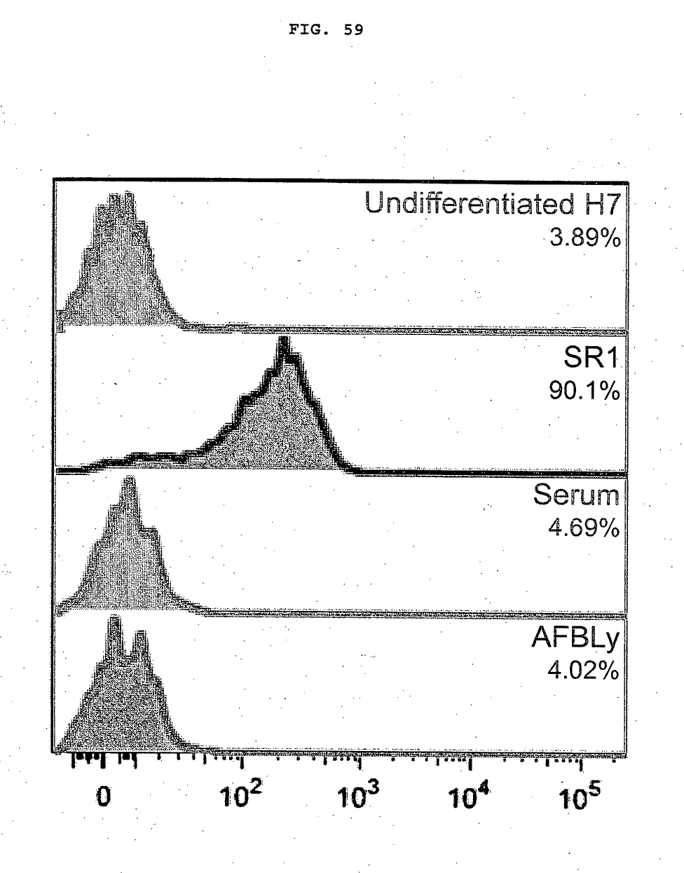

RNA was harvested from adherent cells grown in individual wells of a 12-well plate by the addition of 350 μL of RLT Buffer for several minutes. RNA could be indefinitely frozen at −80° C. or could be directly extracted. RNA extraction was conducted with the RNeasy Micro Kit (Qiagen) generally as per the manufacturer's recommendations with an intermediate 1 hour on-column DNase digestion to eliminate residual genomic DNA, and RNA was finally eluted from the column in 30 μL H20. After assessment of total RNA concentration, generally 100-500 ng of total RNA was used for reverse transcription (Superscript Reverse Transcriptase, Invitrogen) as per the manufacturer's instructions. Finally, cDNA was diluted 1:30 in H₂O and was used for qPCR in 384-well high-throughput format. For each individual qPCR reaction per′ well (10 μL), 5 μL of 2×SYBR Green Master Mix (Applied Biosystems) was used and combined with 0.4 μL of combined forward and reverse primer mix (at 10 μM of forward+reverse primers in the combined primer mix). qPCR was conducted for 40 cycles at Tm=60° C., and a dissociation curve was generated at the end of the reaction to ensure only one product was specifically amplified per primer pair. qPCR analysis was conducted by the ddCt method: for each cDNA sample, the expression of experimental genes was internally normalized to the expression of a human housekeeping gene (Pbgd) for that same cDNA sample, and afterwards, expression of experimental genes could be determined between different cDNA samples. For all differentiated populations, expression of experimental genes was compared to undifferentiated hESCs plated for the same experimental set to ensure that any perceived increase or decrease of gene expression was significant relative to the ab initio expression of that gene in undifferentiated hESCs. Thus, for all qPCR data both in matrices (FIG. 1-4) and histograms (FIG. 39-65) all gene expression is normalized such that the level of gene expression (e.g., for SOX17) in hESCs=1. For each experiment, at least two distinct wells per, condition were harvested, and for each well, 2 or 3 technical replicates were performed for each gene whose expression was analyzed by qPCR.

“Undetermined” values were assigned a CT value of 40, thus providing a conservative overestimation of the expression of that gene and thus conservatively underestimating the fold chance between undetermined values and samples that reached a determined value. All qPCR primer pairs (sequences provided in Table 1) were extensively validated to ensure linearity of qPCR product amplification.

TABLE 1
List of developmental marker genes, embryonic
expression domains and gene−specific qPCR primers

| | Gastrulation | | | AP patterning | | |
|---|---|---|---|---|---|---|
| Gene, Primer Sequence & Notes | PS | DE | Mes | FG | MG | HG |
| Pbgd (Housekeeping) | + | + | + | + | + | + |
| F: GGAGCCATGTCTGGTAACGG | | | | | | |
| R: CCACGCGAATCACTCTCATCT | | | | | | |
| Oct4/Pou5f1(and EPI) | + | − | + | − | − | − |
| F: AGTGAGAGGCAACCTGGAGA | | | | | | |
| R: ACACTCGGACCACATCCTTC | | | | | | |
| Sox2(and EPI, NE) | − | − | − | + | − | − |
| F: TGGACAGTTACGCGCACAT | | | | | | |
| R: CGAGTAGGACATGCTGTAGGT | | | | | | |
| Cripto(and EPI) | + | − | + | | | |
| F: TGACCGCTGTGACCCGAAAGACT | | | | | | |
| R: AGTGCGCAGGGCAGCAAGAGTA | | | | | | |
| Brachyury | + | − | + | − | − | − |
| F: TGCTTCCCTGAGACCCAGTT | | | | | | |
| R: GATCACTTCTTTCCTTTGCATCAAG | | | | | | |
| Eomes | Ant | − | Ant | − | − | − |
| F: CAACATAAACGGACTCAATCCCA | | | | | | |
| R: ACCACCTCTACGAACACATTGT | | | | | | |
| Mixl1 | + | − | + | − | − | − |
| F: GGTACCCCGACATCCACTTG | | | | | | |
| R: TAATCTCCGGCCTAGCCAAA | | | | | | |
| Wnt3 | + | − | + | − | − | |
| F: TGACTTCGGCGTGTTAGTGTC | | | | | | |
| R: ATGTGGTCCAGGATAGTCGTG | | | | | | |
| Lhx1/Lim1 | Ant | − | + | − | − | − |
| F: CATCCTGGACCGCTTTCTCT | | | | | | |
| R: CACATCATGCAGGTGAAGCA | | | | | | |
| Tbx6 | + | − | + | − | − | − |
| F: AAGTACCAACCCCGCATACA | | | | | | |
| R: TAGGCTGTCACGGAGATGAA | | | | | | |
| Mesp2 | + | − | + | − | − | − |
| F: AGCTTGGGTGCCTCCTTATT | | | | | | |
| R: TGCTTCCCTGAAAGACATCA | | | | | | |
| Foxa1 | Ant | + | − | + | + | + |
| F: AAGGCATACGAACAGGCACTG | | | | | | |
| R: TACACACCTTGGTAGTACGCC | | | | | | |
| Foxa2 | Ant | + | + | + | + | + |
| F: GGGAGCGGTGAAGATGGA | | | | | | |
| R: TCATGTTGCTCACGGAGGAGTA | | | | | | |
| Gsc | Ant | − | + | − | − | − |
| F: GAGGAGAAAGTGGAGGTCTGGTT | | | | | | |
| R: CTCTGATGAGGACCGCTTCTG | | | | | | |
| Hhex | Ant | + | − | + | − | − |
| F: CACCCGACGCCCTTTTACAT | | | | | | |
| R: GAAGGCTGGATGGATCGGC | | | | | | |
| FzdB/Frizzled8 | Ant | + | ? | | | |
| F: ATCGGCTACAACTACACCTACA | | | | | | |
| R: GTACATGCTGCACAGGAAGAA | | | | | | |
| Evx1 | Pst | − | + | − | − | + |
| F: AGTGACCAGATGCGTCGTTAC | | | | | | |
| R: TGGTTTCCGGCAGGTTTAG | | | | | | |
| Mesp1 | Pst | − | + | − | − | − |
| F: GAAGTGGTTCCTTGGCAGAC | | | | | | |
| R: TCCTGCTTGCCTCAAAGTGT | | | | | | |
| Cxcr4 | ? | + | + | − | − | − |
| F: CACCGCATCTGGAGAACCA | | | | | | |
| R: GCCCATTTCCTCGGTGTAGTT | | | | | | |
| Cer1 | − | + | + | Ant. | − | − |
| F: TTCTCAGGGGGTCATCTTGC | | | | | | |
| R: ATGAACAGACCCGCATTTCC | | | | | | |
| Sox17(and ExEn) | − | + | − | Pst | + | + |
| F: CGCACGGAATTTGAACAGTA | | | | | | |
| R: GGATCAGGGACCTGTCACAC | | | | | | |
| Isl1 | − | − | + | + | ? | ? |
| F: AGATTATATCAGGTTGTACGGGATCA | | | | | | |
| R: ACACAGCGGAAACACTCGAT | | | | | | |
| Nkx2.5 | − | − | + | Ant. | − | − |
| F: CAAGTGTGCGTCTGCCTTT | | | | | | |
| R: CAGCTCTTTCTTTTCGGCTCTA | | | | | | |
| Foxc1 | − | − | + | | | |
| F: ACTCGGTGCGGGAGATGTTCGAGT | | | | | | |
| R: AAAGCTCCGGACGTGCGGTACAGA | | | | | | |
| Foxf1 | + | − | + | − | − | − |
| F: AGCAGCCGTATCTGCACCAGAA | | | | | | |
| R: CTCCTTTCGGTCACACATGCTG | | | | | | |
| Irx3 | − | − | + | Ant. | − | − |
| F: CTCCGCACCTGCTGGGACTTC | | | | | | |
| R: CTCCACTTCCAAGGCACTACAG | | | | | | |
| Hand1 | − | − | + | − | − | − |
| F: GTGCGTCCTTTAATCCTCTTC | | | | | | |
| R: GTGAGAGCAAGCGGAAAAG | | | | | | |
| Snai2/Slug | − | − | + | − | − | − |
| F: ATCTGCGGCAAGGCGTTTTCCA | | | | | | |
| R: GAGCCCTCAGATTTGACCTGTC | | | | | | |
| Mnx1/Hb1x9/Hb9(Dorsal DE) | − | − | − | + | + | + |
| F: TAAGATGCCCGACTTCAACTCCCAGGC | | | | | | |
| R: TGGGCCGCGACAGGTACTTGTTGA | | | | | | |
| Otx2 | − | − | + | Ant | − | − |
| F: GGAAGCACTGTTTGCCAAGACC | | | | | | |
| R: CTGTTGTTGGCGGCACTTAGCT | | | | | | |
| Pax9 | − | − | + | Ant | − | + |
| F: TGGTTATGTTGCTGGACATGGGTG | | | | | | |
| R: GGAAGCCGTGACAGAATGACTACCT | | | | | | |
| Tbx1 | − | − | + | Ant | − | − |
| F: CGGCTCCTACGACTATTGCCC | | | | | | |
| R: GGAACGTATTCCTTGCTTGCCCT | | | | | | |
| Odd1 | − | − | + | Pst | − | − |
| F: CAGCTCACCAACTACTCCTTCCTTCA | | | | | | |
| R: TGCAACGCGCTGAAACCATACA | | | | | | |
| Hnf6/Onecut1 | − | − | + | Pst | − | Tns |
| F: CCCACCGACAAGATGCTCAC | | | | | | |
| R: GCCCTGAATTACTTCCATTGCTG | | | | | | |
| Hnf1b/vHnf1/Tcf2 | − | − | − | Pst | + | + |
| F: AGGCCACAATCTCCTCTCAC | | | | | | |
| R: TTGCTGGGGATTATGGTGGGA | | | | | | |
| Hnf4α | − | − | − | Pst | + | Low |
| F: CATGGCCAAGATTGACAACCT | | | | | | |
| R: TTCCCATATGTTCCTGCATCAG | | | | | | |
| Afp | − | − | − | Pst | + | ? |
| F: CTTTGGGCTGCTCGCTATGA | | | | | | |
| R: GCATGTTGATTTAACAAGCTGCT | | | | | | |
| Alb1/Albumin | − | − | − | Pst | − | − |
| F: ACCCCACACGCCTTTGGCACAA | | | | | | |
| R: CACACCCCTGGAATAAGCCGAGCT | | | | | | |
| Transthyretin/Ttr | − | − | − | Pst | − | − |
| F: GCTGGGAGCAGCCATCACAGAAGT | | | | | | |
| R: CACTTGGATTCACCGGTGCCCGTA | | | | | | |
| Hoxa1/Hox1.6 | ? | − | + | Pst | ? | ? |
| F: CGTGAGAAGGAGGGTCTCTTG | | | | | | |
| R: GTGGGAGGTAGTCAGAGTGTC | | | | | | |
| Hoxa3/Hox1.5 | + | − | + | Pst | − | − |
| F: AGCAGCTCCAGCTCAGGCGAAA | | | | | | |
| R: TGGCGCTCAGTGAGGTTCAG | | | | | | |
| Hoxb4 | − | − | + | + | − | − |
| F: GTTCCCTCCATGCGAGGAATA | | | | | | |
| R: GCTGGGTAGGTAATCGCTCTG | | | | | | |
| Hoxc5 | − | − | ? | Pst | + | − |
| F: GCAGAGCCCCAATATCCCTG | | | | | | |
| R: CCGATCCATAGTTCCCACAAGTT | | | | | | |
| Hoxb6 | − | − | + | − | + | − |
| F: TCCTATTTCGTGAACTCCACCT | | | | | | |
| R: CGCGGGGTAATGTCTCAGC | | | | | | |
| Hoxc6 | − | − | | − | + | − |
| F: ACCCCTGGATGCAGCGAATGAATTCG | | | | | | |
| R: GTTCCAGGGTCTGGTACCGCGAGTA | | | | | | |
| Hoxb8 | − | − | + | − | + | + |
| F: GACCCCGGCAATTTCTACGG | | | | | | |
| R: CGCACCGAATAGGCTCTGG | | | | | | |
| Hoxd13/Hoxd4.8 | − | − | + | − | − | Pst |
| F: ACCAGCCACAGGGGTCCCACTTTT | | | | | | |
| R: ACGCCGCCGCTTGTCCTTGTTA | | | | | | |
| Cdx2 | − | − | + | Pst | + | + |
| F: GGGCTCTCTGAGAGGCAGGT | | | | | | |
| R: CCTTTGCTCTGCGGTTCTG | | | | | | |
| Pdgfrα (and ExEn) | ? | − | + | | | |
| F: CCGTGGGCACGCTCTTTACTCCATGT | | | | | | |
| R: GGATTAGGCTCAGCCCTGTGAGAAGAC | | | | | | |
| Snail/Slug | + | − | + | | | |
| F: CCGACCCCAATCGGAAGCCTAACT | | | | | | |
| R: AGTCCCAGATGAGCATTGGCAGCGAG | | | | | | |
| Pdx1 | − | − | − | Pst | − | − |
| F: GCGTTGTTTGTGGCTGTTGCGCA | | | | | | |
| R: AGCTTCCCCGCTGTGTGTGTTAGG | | | | | | |
| Pax6(and NE) | - | − | − | Pst | − | − |
| F: GCAGATGCAAAAGTCCAGGTG | | | | | | |
| R: CAGGTTGCGAAGAACTCTGTTT | | | | | | |
| Oct6/Pou3f1(and NE) | − | − | − | − | − | − |
| F: CAGAAGGAGAAGCGCATGACCC | | | | | | |
| R: CTAGCTCCCCAGGCGCGTA | | | | | | |
| Sox7 (and ExEn) | − | − | − | | | |
| F: ACGCCGAGCTCAGCAAGAT | | | | | | |
| R: TCCACGTACGGCCTCTTCTG | | | | | | |
| Onecut2 | − | − | − | − | Pst | − |
| F: CGATCTTTGCGCAGAGGGTGCTGT | | | | | | |
| R: TTTGCACGCTGCCAGGCGTAAG | | | | | | |
| E−cadherin/Cdh1(and EPI) | − | + | − | | | |
| F: AGCCCTTACTGCCCCCAGAG | | | | | | |
| R: GGGAAGATACCGGGGGACAC | | | | | | |
| N−cadherin/Cdh2 | + | − | + | | | |
| F: CAACGGGGACTGCACAGATG | | | | | | |
| R: TGTTTGGCCTGGCGTTCTTT | | | | | | |

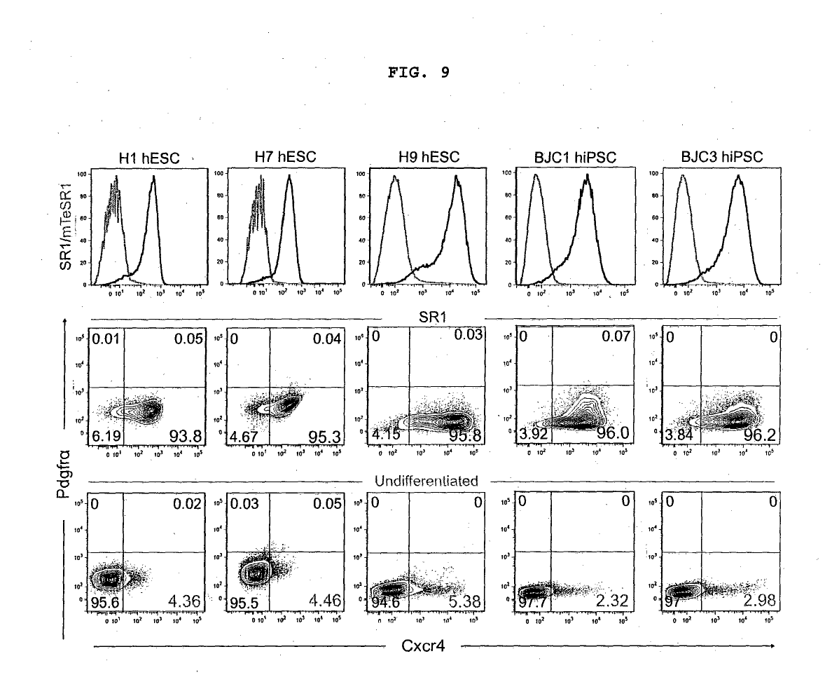

To deduce the developmental signaling logic underlying cell-fate bifurcations, signaling-perturbation matrices (FIG. 1-23) were generated to visually represent qPCR data of developmental gene expression (rows) in response to various signaling-perturbations (columns). Signaling-perturbation matrices were generated using GenePattern's HeatMapViewer module (http://genepattern.broadinstitute.org), using as input data matrices of signaling-perturbation qPCR responses that were normalized to levels of developmental gene expression in undifferentiated hESC as described above. In HeatMapViewer, gene expression values are linearly transformed into colors (as indicated by the color legend below each matrix) in which no color represents low gene expression, stronger color represents higher gene expression and the strongest shade of color is equivalent to the highest level of the gene that was expressed in all signaling-perturbations tested in that matrix.

Single-Cell qPCR

Individual undifferentiated H7 hESC or those differentiated by SR1, AFBLy or serum regimens for 48 hours were manually picked using a mouth pipette (20 cells per condition, for a total of 80 cells overall). They were then lysed and RNA from individual cells was subject to reverse transcription and targeted preamplification using pooled specific primer pairs (for Actb, Yuhazi, Pbgd, Blimp1, Foxa2, Gata6, Sox17, Shisa2, Mixl1, Gata4, Mesp2, Pdgfrα, Oct4, Sox2, Nanog and Prdm14; Table 2) using the CellsDirect One-Step qRT-PCR Kit (Life Technologies, 11753-500).

TABLE 2
List of primers for single−cell qPCR

| Gene name | Primer sequence |
|---|---|
| β−Actin/Actb | F: TTT GAA TGA TGA GCC TTC GTG CCC |
| | R: GGT CTC AAG TCA GTG TAC AGG TAA GC |
| Pbgd | F: GGAGCCATGTCTGGTAACGG |
| | R: CCACGCGAATCACTCTCATCT |
| Yuhazi | F: TGCAAAGACAGCTTTTGATGAAGCC |
| | R: AGAATGAGGCAGACAAAAGTTGGAA |
| Blimp1/Prdm1 | F: TCTCCAATCTGAAGGTCCACCTG |
| | R: GATTGCTGGTGCTGCTAAATCTCTT |
| Foxa2 | F: GGGAGCGGTGAAGATGGA |
| | R: TCATGTTGCTCACGGAGGAGTA |
| Gata6 | F: ATGCTTGTGGACTCTACATGAAACT |
| | R: TGCTATTACCAGAGCAAGTCTTTGA |
| Shisa2 | F: TTCCTTTACTGAAGGGAGACGAAGG |
| | R: CCATCCAAAGGAATCGTGCCATAAA |
| Sox17 | F: CGCACGGAATTTGAACAGTA |
| | R: GGATCAGGGACCTGTCACAC |
| Mixl1 | F: TACCCCGACATCCACTTGCG |
| | R: GGTTGGAAGGATTTCCCACTCTGA |
| Gata4 | F: CGGAAGCCCAAGAACCTGAATAAAT |
| | R: ACTGAGAACGTCTGGGACACG |
| Mesp2 | F: AGCTTGGGTGCCTCCTTATT |
| | R: TGCTTCCCTGAAAGACATCA |
| Pdgfrα | F: CCGTGGGCACGCTCTTTACTCCATGT |
| | R: GGATTAGGCTCAGCCCTGTGAGAAGAC |
| Prdm14 | F: GCTTCGGATCCACATTCTTCATGTT |
| | R: TGGAGGCTGTGAACCTCTTAGTACA |
| Sox2 | F: AGTGTTTGCAAAAGGGGGAAAGTAG |
| | R: CCGCCGCCGATGATTGTTATTATT |
| Nanog | F: AGAACTCTCCAACATCCTGAACCTC |
| | R: CTGAGGCCTTCTGCGTCACA |
| Oct4 | F: AGTGAGAGGCAACCTGGAGA |
| | R: ACACTCGGACCACATCCTTC |

Prior to this assay, primer pairs were rigorously validated for linear amplification and for their lack of signal in a no template control (NTC). After preamplification, unused primers were removed in a cleanup step using Exonuclease I (New England BioLabs, PN M0293) and resultant cDNA from individual cells was prepared for high-throughput qPCR in a Biomark 96.96 Dynamic Array (Fluidigm) on a Biomark HD System (Fluidigm) using the indicated primer pairs and SsoFast, EvaGreen Supermix with Low ROX (Bio-Rad). Subsequently, Ct values were internally normalized to Yuhazi expression for each single cell and individual clones displaying deviant housekeeping gene expression were typically excluded from downstream analyses. Single-cell qPCR data were visualized as a gene expression heatmap using GenePattern's HeatMapViewer module (http://genepattern.broadinstitute.org). To determine cells expressing significant Foxa2 levels, after all Ct values were internally normalized to Yuhazi (such that dCtYuhazi=0 for all cells), any cells with dCtFoxa2<6.5 were regarded Foxa2+. At this cutoff, no hESC (20/20) expressed Foxa2, whereas all SR1-differentiated cells (20/20) expressed Foxa2 and few AFBLy- or serum-induced cells (1/20 and 2/20, respectively) expressed Foxa2.

### Fluorescence-Activated Cell Sorting (FACS) Analysis

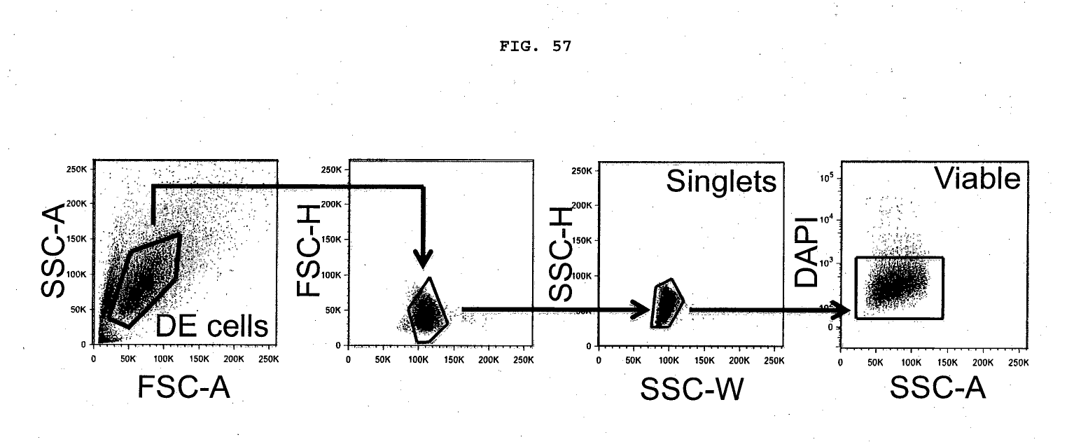

SR1-differentiated or undifferentiated hPSC in 6-well format were washed (DMEM/F12), briefly treated with TrypLE Express (Gibco, 0.75 mL/well in a 6-well plate) and vigorously tapped to detach cells. Cells in TrypLE were collected and subsequently, wells were washed multiple times with FACS buffer (PBS+0.5% BSA+5 mM EDTA) to collect residual cells and, thoroughly triturated to yield a single-cell suspension. The cell suspension was centrifuged (5 mins), resuspended in FACS buffer (30-50 μL/individual stain), and stained with anti-Cxcr4 PE Cy7 (BD Biosciences, 560669, diluted 1:5) and/or anti-Pdgfrα PE (BD Biosciences, 556005, diluted 1:50) for 30 minutes on ice in the dark. Subsequently, cells were washed twice in FACS buffer (1.5 mL/individual stain) and collected by centrifugation (5 mins). Finally, washed cells were resuspended in FACS buffer (300 μL/individual stain), filtered (40 μm filter, BD Biosciences), stained for several minutes with DAPI (to assess cell viability) and were analyzed on a FACSAria II (Stanford Stem Cell Institute FACS Core Facility). Digital compensation was performed to control for channel bleedthrough and gates were rigorously set based on fluorescence minus one (FMO) controls. Undifferentiated hPSC and SR1-differentiated cells were always identically stained and analyzed in parallel in the same experiment to ensure specificity of antibody staining. A minimum of 10,000 events were analyzed for each individual stain, and subsequently, events were parsed by virtue of FSC-A/SSC-A analysis; cell singlets were selected by gating on FSC-W/FSC-H followed by SSC-H/SSC-W; and finally dead cells were excluded by gating only on DAPI-cells (gating strategy represented in FIG. 57). Optionally, cells were costained with anti-CD90 FITC (BD Biosciences, 555595, diluted 1:50) as per above (for FIG. 56), as CD90 identifies undifferentiated hPSC (e.g., Drukker et al., 2012; Tang et al., 2011).

hPSC-derived DE was defined as Cxcr4+Pdgfrα− on the basis of the respective embryological expression domains of these cell-surface markers. Although Cxcr4+ alone is typically used to assign DE during hPSC differentiation (D'Amour et al., 2005), Cxcr4 is expressed also in extraembryonic endoderm as well as subtypes of mesoderm in vivo in the vertebrate gastrula, including extraembryonic and intraembryonic mesoderm (Drukker et al., 2012; McGrath et al., 1999). Thus, Cxcr4+ alone is not suitable to precisely define DE during hPSC differentiation (as argued by Drukker et al., 2012). However, Pdgfrα is expressed in extraembryonic endoderm (both pre-implantation and post-implantation), including both visceral and parietal endoderm and additionally Pdgfrα is broadly expressed in early intraembryonic and extraembryonic mesoderm in vivo (Orr-Urtreger et al., 1992; Plusa et al., 2008). Thus, together Cxcr4+Pdgfrα− more accurately delineates DE by excluding potential mesoderm or extraembryonic endoderm.

To precisely quantify APS and DE differentiation efficiencies MIXL1-GFP HES3 (Davis et al., 2008) and SOX17-mCHERRY H9 knock-in reporter lines (described below) were respectively employed in which fluorescent reporters had been introduced into the indicated loci through homologous recombination. After 24 hours of differentiation in SR1 (APS) or 48 hours of differentiation in SR1 (DE), differentiated and undifferentiated reporter hESC were dissociated into single cells and analyzed by flow cytometry as per above. To determine the number of MIXL1-GFP+ or SOX17-mCHERRY+ cells after respective differentiation treatments, gating was rigorously set based on expression of these reporters in undifferentiated hESC that were analyzed in parallel: in all instances, gates were set such that less than 1-2% of undifferentiated hESC were MIXL1-GFP+ or SOX17-mCHERRY+.

Generation of the Sox17^{mCHERRY/w} hESC Reporter Line

The SOX17-mCHERRY targeting vector comprised an 8.3kb 5′ homology arm that encompassed genomic sequences located immediately upstream of the Sox17 translational start site, sequences encoding mCHERRY (Shaner et al., 2004), a loxP-flanked PGK-Neo antibiotic resistance cassette and a 3.6kb 3′ SOX17 homology arm (L Azolla, EG Stanley and AG Elefanty, unpublished results). The H9 hESC line was electroporated with the linearized vector and correctly targeted clones identified using a PCR based screening strategy (Costa et al., 2007). The antibiotic resistance cassette was excised using Cre recombinase. The SOX17^{mCHERRY/w} hESC reporter line used (referred to as SOX17-mCHERRY throughout this paper) was validated by demonstrating the correlation between SOX17 RNA and protein and mCHERRY expression on populations of FACS-sorted cells (L Azolla, ES Ng, EG Stanley and AG Elefanty, manuscript in preparation).

### Deep Transcriptome Sequencing (RNA-Seq)

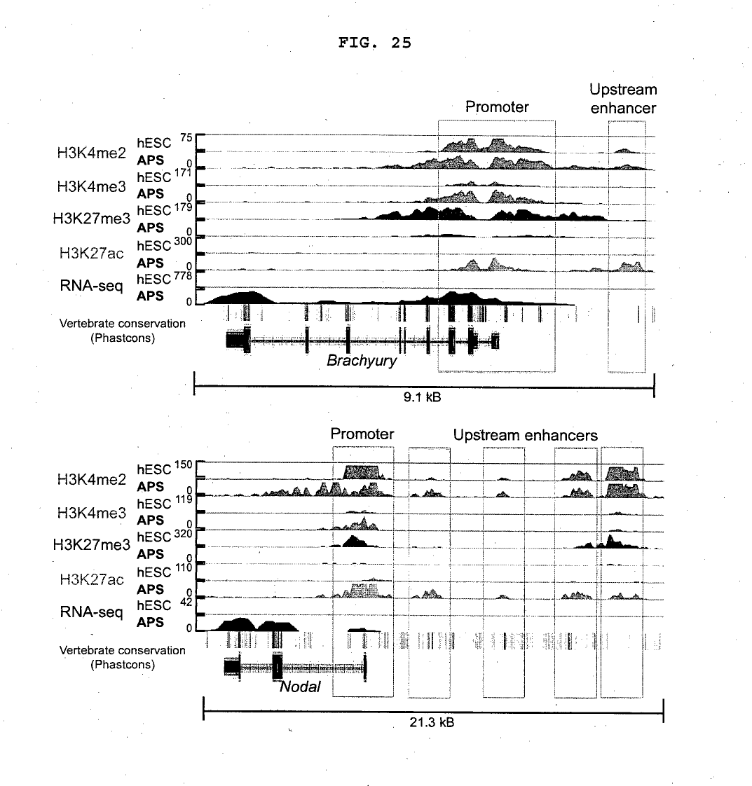

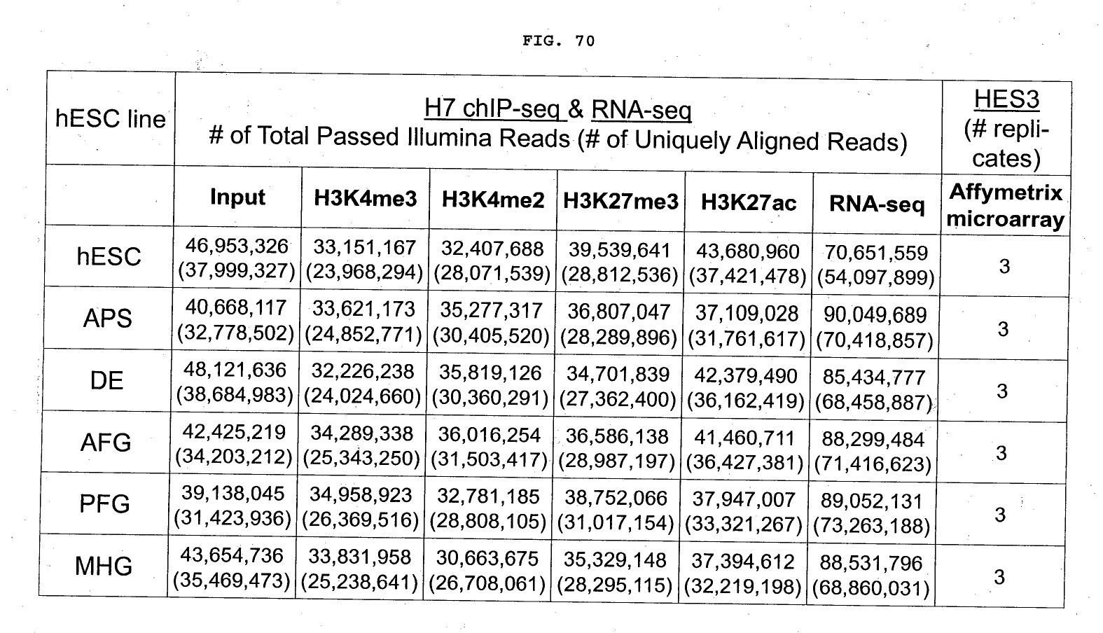

Total cellular RNA for each lineage was extracted as described above (RNeasy Micro Kit, Qiagen) and 1 μg of total RNA was used to prepare each individual RNA-seq library. RNA-seq library construction was conducted with the TruSeq RNA Library Preparation. Kit (Illumina) as per the manufacturer's instructions. In brief, total RNA was poly-A selected twice, fragmented to 300-500 bp by chemical- and heat-induced scission, end-repaired and 3′ adenylated. Thereafter, adapter ligation was performed and libraries were PCR amplified by primers directed against the adapters (15 cycles). After library construction, insert size was assessed by on-chip electrophoresis (Agilent Bioanalyzer) and readable fragments were quantified by qPCR with primers directed against, the adapters. Libraries were multiplexed such that two RNA-seq libraries were assessed per individual Hi-Seq lane. High-throughput sequencing was conducted on the Hi-Seq 2000 (Illumina) by the Genome Institute of Singapore's Solexa Group for 1×36+7 cycles (single read, 36 bp of insert of a multiplexed library, 7 bp for adapter barcode identification). RNA-seq reads were mapped to the hg19 human reference genome using TopHat (Trapnell et al., 2009). Aligned reads were assembled and FPKM (fragments per kilobase of exon per million mapped reads) calculated using Cufflinks. Genes with expression values of FPKM>1 were selected for subsequent analyses. FPKM values were log transformed [log 2(FPKM+1)] and lineage-specific genes were defined as log 2(FPKM+1)>2 across all lineages (FIG. 24). Library sequencing statistics are provided in FIG. 70.

### Microarray Analysis

For each biological condition, four biological replicates were produced by hESC differentiation (HES3 hESC line), RNA was extracted (RNeasy Micro Kit, Qiagen as per above), and RNA quality was assessed by Bioanalyzer on-chip electrophoresis (Agilent). Only samples with an RNA integrity (RIN) value>9.5 were used for microarray analysis and eventually the three biological replicates with the highest RNA quality were chosen for microarray analysis, which was conducted by the Stanford PAN Microarray Core (Elizabeth Guo) by hybridization to the Affymetrix Human Genome U133 Plus 2.0 Array. Raw data (.cel files) were exported and uploaded to the Broad Institute's GenePattern online platform (http://genepattern.broadinstitute.org), converted (ExpressionFileCreator module), preprocessed (PreprocessDataset module, floor threshold=20, ceiling threshold=20,000, minimum fold change between datasets examined=3), and heat maps were created thereof (HeatMapViewer module).

For analysis of AFBLy differentiation in the H9 hESC line conducted by an independent laboratory (Touboul et al., 2010), raw microarray data from that study were downloaded from the ArrayExpress repository (http://www.ebi.ac.uk/microarray-as/ae/, accession number E-MEXP-2373) and analyzed using GeneSpring GX software. Raw microarray data were normalized and processed as per standard procedure, and finally, of all genes detected by microarray to be minimally expressed in at least one population, expression data of undifferentiated H9 hESCs and AFBLy-differentiated hESCs were compared. Genes differentially expressed (>2.0-fold change) between AFBLy-differentiated and undifferentiated hESCs were enumerated and the function of AFBLy-upregulated genes was unbiasedly ascertained by DAVID/EASE assignment of gene ontology terms (http://david.abcc.ncifcrf.gov/) under the background “HumanRef-8_V3_{—}0_R2_{—}11282963_A”.

### Immunochemistry

Adherent cells were washed once with PBS (Gibco), fixed in 4% paraformaldehyde (in PBS) for 15 minutes at room temperature, and washed twice (with PBS). Fixed cells were simultaneously blocked and permeabilized in blocking solution (5% donkey serum+0.1% Triton X100 in PBS) for 1 hour at 4° C. and washed twice (PBS). Primary antibody staining was conducted with primary antibody diluted in blocking buffer overnight at 4° C. Afterwards, cells were washed twice (PBS). Secondary antibody staining was conducted in blocking buffer for 1 hour at 4° C. Afterwards, the secondary antibody was removed and nuclear counterstaining was conducted with DAPI (Invitrogen Molecular Probes, diluted in PBS) for 5 minutes at room temperature. Cells were washed three times in PBS to remove excess antibody and DAPI, and fluorescence microscopy was conducted with a Zeiss Observer D1. Antibodies and effective concentrations are provided in Table 3.

TABLE 3
List of antibodies for inununocytochemistry

| Antibody | Supplier/Catalog No. | Effective Dilution |
|---|---|---|
| Rabbit α-Eomes | Abcam, ab23345 | 1:300 |
| Rabbit α-Foxa2 | Upstate, 07-633 | 1:200 |
| Goat α-Sox17 | R&D Systems, AF1924 | 1:1000 (0.2 μg/mL) |
| Goat α-Foxa2 | R&D Systems, AF2400 | 1:500 (0.4 μg/mL) |
| Goat α-Brachyury | R&D Systems, AF2085 | 1:250 (0.4 μg/mL) |
| Mouse α-Lhx1 | R&D Systems, MAB2725 | 1:500 (0.4 μg/mL) |
| Goat α-Cdx2 | R&D Systems, AF3665 | 1:100 |
| Rabbit α-Afp | Dako, A000829 | 1:100 |
| Goat α-Otx2 | R&D Systems, AF1979 | 1:100 |

### Western Blotting

Samples were separated by SDS-PAGE and transferred on a PVDF membrane (100V at 4° C., for 1 hour). Membranes were blocked in TEST+575 milk for 1 hour at room temperature followed by incubation with goat anti-Sox17 (R&D Systems, AF1924) or mouse anti-Foxa1 (Abcam, ab55178) primary antibodies (1:1000) or anti-β-Actin (Santa Cruz, 1:5000) primary antibody for 1 hour at room temperature. β-Actin was used as an internal loading control. Membranes were washed 5× in TEST and incubated for 1 hour with goat anti-mouse (Jackson ImmunoResearch, 1:5000) or donkey anti-goat (Santa Cruz, 1:2000) HRP-conjugated IgG secondary antibodies. After washing in TBST, proteins were detected using ECL Prime (GE Healthcare).

Transplantation of hESC-Derived Hepatic Progeny and Subsequent Analysis

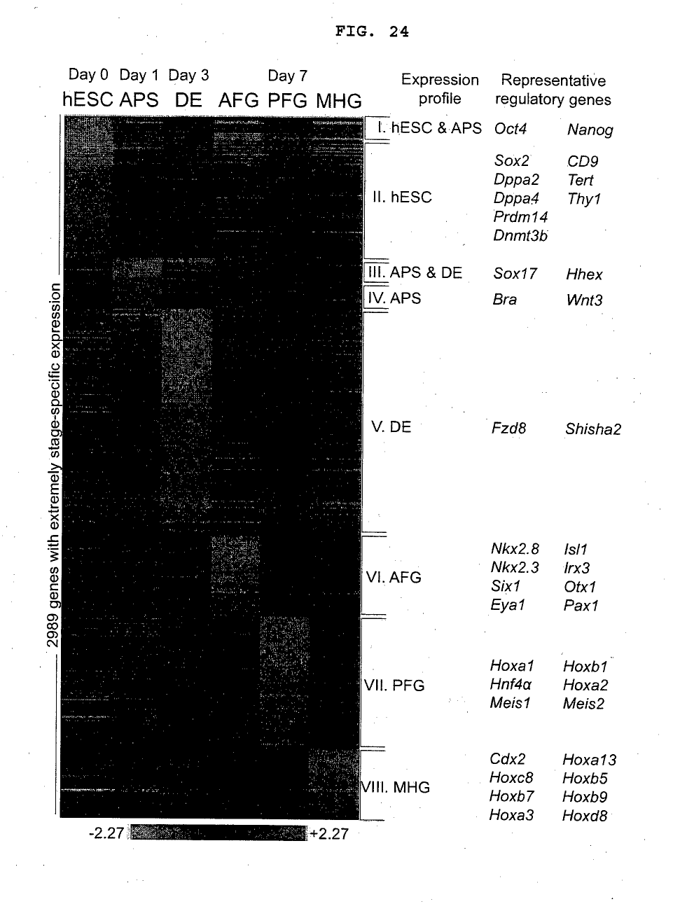

H7 hESC were stably transfected with a constitutively active CAG-GFP vector to indelibly label them and their progeny with GFP. Using SR1, they were differentiated into early Afp+ hepatic progenitors as described above (day 6-7 of differentiation) or were subsequently differentiated into later hepatic progeny using 12 days of further empirical differentiation: 2 days of BMP4 (10 ng/mL) followed by 10 further days of dexamethasone (Sigma, 10 μM) and oncostatin M (10 ng/mL, R&D Systems). Early hESC-differentiated progenitors or later hepatic progeny were dissociated into single cells and 50,000-100,000 cells were transplanted into the liver of a neonatal mouse as previously described (Chen et al., 2013). In brief, newborn immunodeficient NOD-SCID Il2γr−/− mice (but not otherwise genetically conditioned) were sublethally irradiated (100 rads) and hepatic cells were directly transplanted into the liver within 24 hours of birth. 2-3 months later, sera were analyzed by ELISA for presence of human albumin (as described by Chen et al., 2013) and mice were sacrificed. Recipient livers were fixed (formalin), embedded (paraffin), and then sectioned and stained with rabbit anti-human albumin (Abcam, ab2406), mouse anti-GFP (Santa Cruz Biotechnology, sc-9996), mouse anti-HepPar1 (Abcam, ab720) or rabbit anti-Afp (Sigma, HPA010607) to detect hESC-derived hepatic progeny in recipient liver parenchyma. Statistical significance between human albumin serum concentrations in mice transplanted with hESC-derived early hepatic progenitors or later differentiated hepatic cells was assessed by a two-sided Whitney-Mann test (FIG. 23).

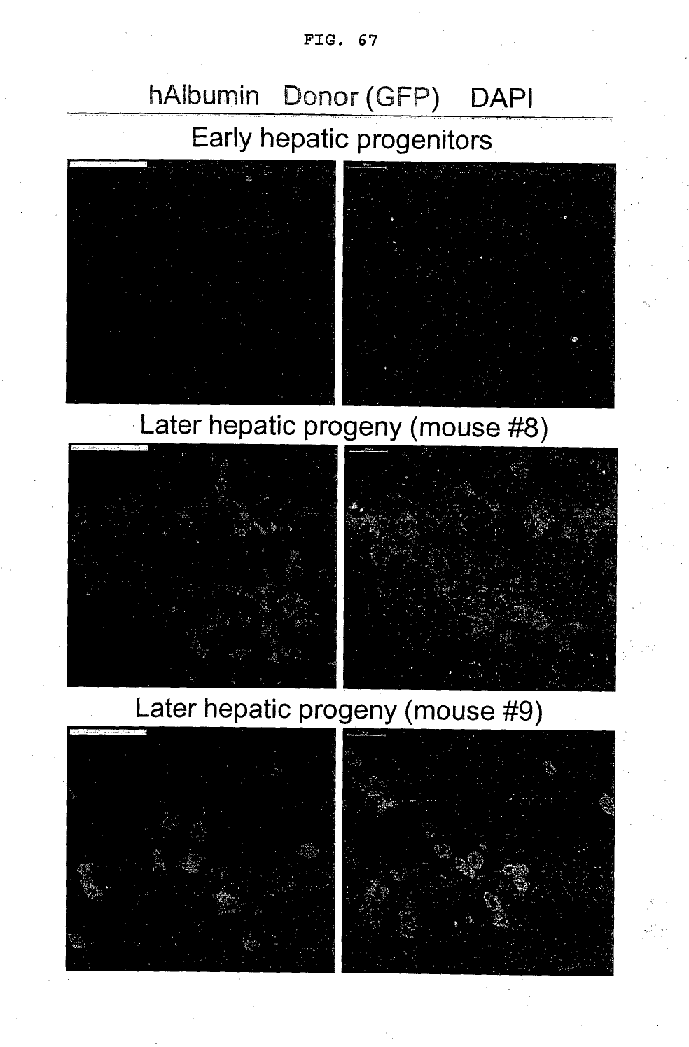

However, for FIG. 67, anti-GFP staining was conducted with rabbit anti-GFP (Abcam, ab290): because the anti-human albumin antibody was also raised in a rabbit background, costaining for both markers could not be performed simultaneously-rather, serial sections were stained with each respective antibody.

### Low-Density Lipoprotein (LDL) Uptake Assay

hESC, HepG2 cells or hESC-derived hepatic progeny were incubated in their respective basal media with the addition of HGF (20 ng/mL) for 24 hours and then their capacity to uptake LDL was assessed using the LDL Uptake Cell-Based Assay Kit (Cayman Chemical, 10011125). In brief, 1:100 LDL-DyLight 594 was added to the respective basal media of all three cell populations for 3 hours at 37° C. Negative controls with no LDL staining were treated in the same way but without the addition of LDL-DyLight 594. Afterwards, cells were fixed and stained for LDLR according to the manufacturers' instructions (Cayman Chemical), with the exception that the anti-LDLR antibody was incubated overnight at 4° C. Cells were visualized by fluorescent microscopy to assess uptake of fluorescent LDL-DyLight 594 and also LDLR expression by immunofluoresecence.

### Cyp3a4 Metabolic Assay

To determine Cyp3a4 enzymatic activity in a luminescent assay, hESC, HepG2 cells or hESC-derived hepatic progeny were briefly washed (PBS) and then treated with their respective basal media containing 3 μM of the bioluminescent Cyp3a4 substrate luciferin-IPA (Promega) for 30-60 minutes at 37° C. Subsequently, 25 μL of medium was transferred to a separate well of a 96-well opaque white luminometer plate, 25 μL of Luciferin Detection Reagent (Promega) was added per well and the plate was incubated for 20 minutes in the dark. A luminometer (Promega GloMax, E9031) was used to record luminescence. Negative control wells containing only basal medium with luciferin-IPA substrate were also recorded to determine technical background.

Cyp3a4 luminescence signals were then normalized to the number of viable cells used in each assay, which was determined using the CellTiter-Glo kit (Promega). Briefly, after hESC, HepG2 cells or hESC-derived progeny were treated with basal medium containing luciferin-IPA, 25 μL of medium was transferred to a separate well of a 96-well opaque white luminometer plate and 25 μL of CellTiter-Glo Reagent was added to each well. After incubation for 2 minutes, luminescence was measured with a luminometer as per above, and Cyp3a4 luminescence assay values (above) were divided by CellTiter-Glo assay values in order to obtain normalized Cyp3a4 activity results. Normalized Cyp3a4 activity results are presented relative to those obtained from undifferentiated hESC.

### Chromatin Immunoprecipitation and Sequencing (ChIP-Seq)

Adherent cells were washed (PBS), fixed in 1% formaldehyde in PBS (10 mins), neutralized with 0.2M glycine (5 mins), collected by scraping, washed (cold PBS supplemented with Complete Protease Inhibitor (Roche)), pelleted, flash frozen (liquid N2), and stored (−80° C.). Prior to immunoprecipitation, fixed cell pellets were thawed, lysed in 1% SDS lysis buffer (50 mM HEPES-KOH pH 7.5, 150 mM NaCl, 1 mM EDTA, 1% Triton X-100, 0.1% Na deoxycholate, 1% SDS with 1× Complete Protease Inhibitor) twice for 30 minutes each time to extract nuclei, and sonicated for 10 cycles at high intensity (30 seconds on, 60 seconds off) in 1% SDS lysis buffer with a pre-cooled Next-Gen Bioruptor (Diagenode). To assess sonication efficiency, a small amount of sonicated chromatin was digested with Proteinase K (1 hour, 50° C.), column-purified, and electrophoresed to confirm that sonication was successful (fragments 100-300 bp in size). Sonicated chromatin was diluted ten times in chIP dilution buffer (0.01% SDS, 1.1% Triton X-100, 1.2 mM EDTA, 16.7 mM Tris-HCl pH 8.1, and 167 mM NaCl) to yield an effective ˜0.1% SDS concentration for immunoprecipitation, centrifuged (13,200 rpm, 10 mins) to remove cellular debris, and pre-cleared overnight with Protein G Dynabeads (Invitrogen).

Concurrently, for each individual chIP, 100 μL of Protein G Dynabeads was washed twice (PBS+0.1% Triton X-100), complexed with ChIP-qualified antibody (Table 4) overnight at 4° C., and washed thrice more to yield antibody-bead complexes. Antibody-bead complexes were added to pre-cleared chromatin.

TABLE 4
List of antibodies for chromatin immunoprecipitation.

| Antibody | Supplier/Catalog No. | Species | Amount per IP | |
|---|---|---|---|---|
| α-H3K4me2 | Abcam, ab32356 (100 ul) | Rabbit | 8 | μL |
| | | IgG | | |
| α-H3K27ac | Abcam, ab4729 (100 μg) | Rabbit | 10 | μg |
| | | IgG | | |
| α-H3K4me3 | Abcam, ab8580 (50 μg) | Rabbit | 10 | μg |
| | | IgG | | |
| α-H3K27me3 | Millipore/Upstate, 07-449 | Rabbit | 10 | ug |
| | (200 μg) | IgG | | |

After overnight immunoprecipitation (4° C.), antigen-antibody-bead complexes were washed twice respectively in low salt wash buffer (0.1% SDS, 1% Triton X-100, 2 mM EDTA, 20 mM Tris pH 8.0, 150 mM NaCl), high salt wash buffer (0.1% SDS, 1% Triton X-100, 2 mM EDTA, 20 mM Tris pH 8.0, 500 mM NaCl), LiCl wash buffer (10 mM Tris pH 8.0, 1 mM EDTA, 0.25M LiCl, 1% Nonidet P-40), and finally, TE buffer. Antibodies were eluted from beads and formaldehyde cross-linking was reversed overnight by mild heating (65 □C), and chromatin was sequentially treated with RNase and Proteinase K before final column purification. The final concentration of immunoprecipitated chromatin was quantified by PicoGreen (Invitrogen).

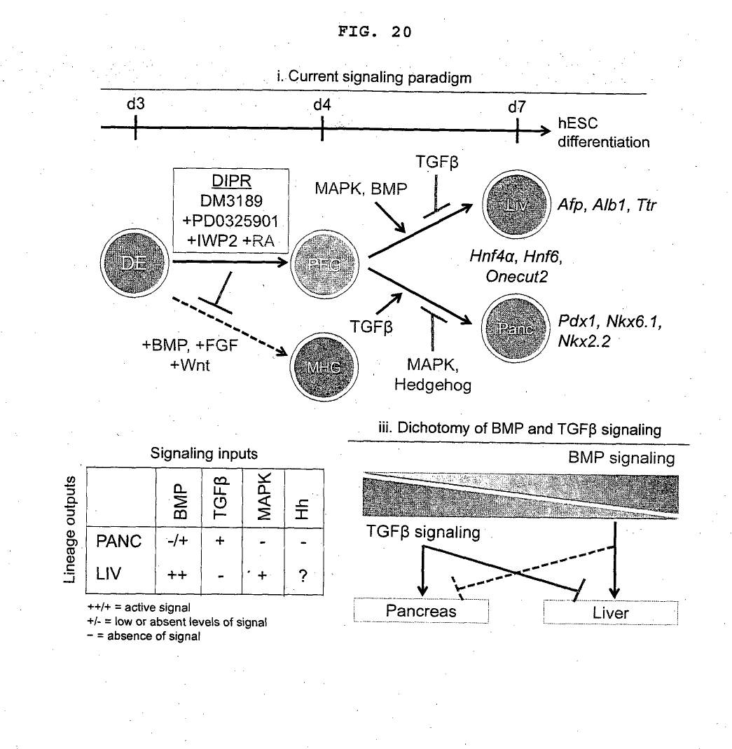

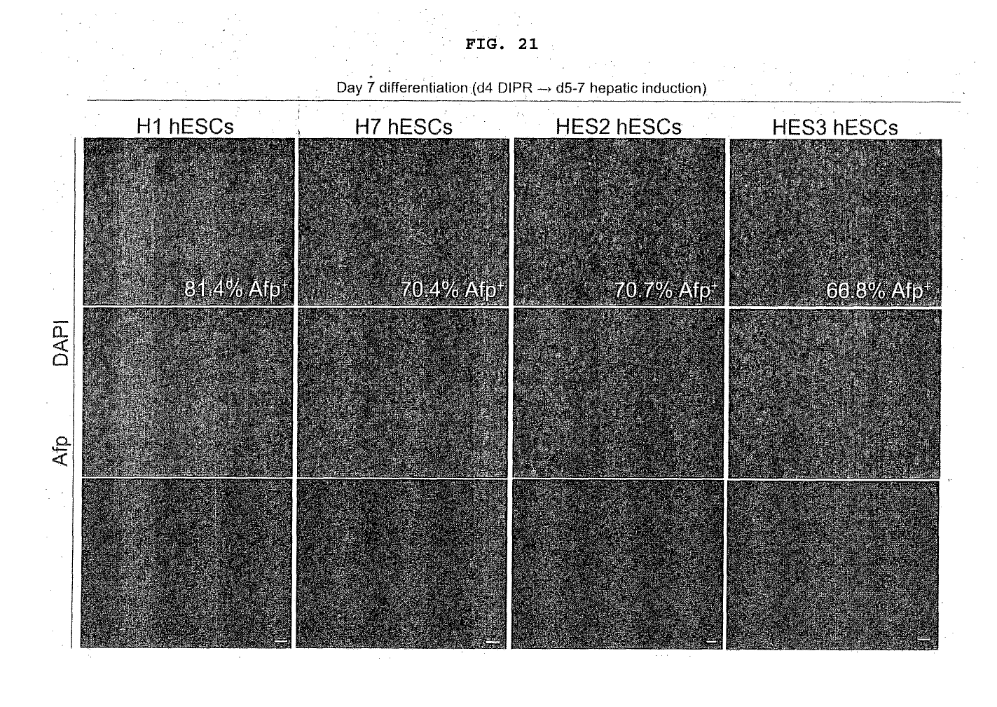

Illumina sequencing libraries were generated using the TruSeq ChIP Sample Preparation Kit (Illumina). Briefly, 10 ng of ChIP-enriched DNA was end-repaired, 3′ adenylated, ligated with Illumina adapters, and amplified through 15 cycles of PCR amplification with Phusion High Fidelity DNA polymerase (Finnzymes) with primers directed against the adapters. After library constructed was completed, insert size was re-verified by on-chip electrophoresis (Agilent Bioanalyzer) and readable fragments were quantified by qPCR with primers directed against the adapters. High-throughput sequencing was conducted on the Hi-Seq 2000 (Illumina) by the Genome Institute of Singapore's Solexa Group for 1×36+7 cycles (single read, 36 bp of insert of a multiplexed library, 7 bp for adapter barcode identification). Sequenced reads were mapped to the hg19 human reference genome using Bowtie (Langmead et al., 2009), allowing up to 3 by mismatches and discarding reads mapping to more than 1 genomic locus. Each aligned fragment was extended by 200 bp and input-normalization was performed using MACS (Zhang et al., 2008). Histone peak visualization was performed using the Integrative Genomics Viewer from the Broad Institute (Thorvaldsdottir et al., 2012). Library sequencing statistics are provided in FIG. 20.

### Assigning and Analyzing Enhancers During ChIP-Seq Analysis

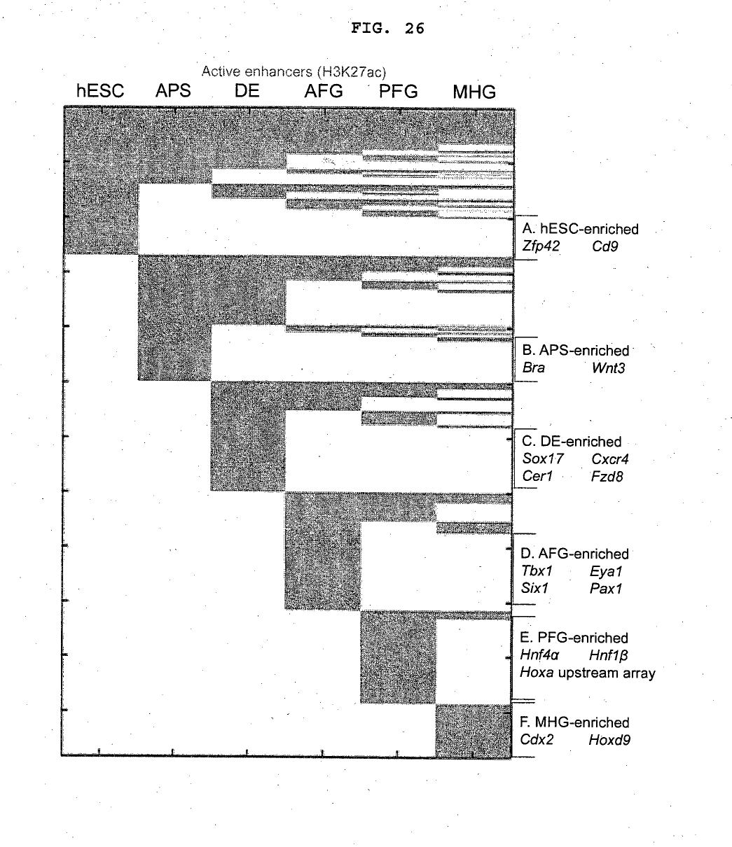

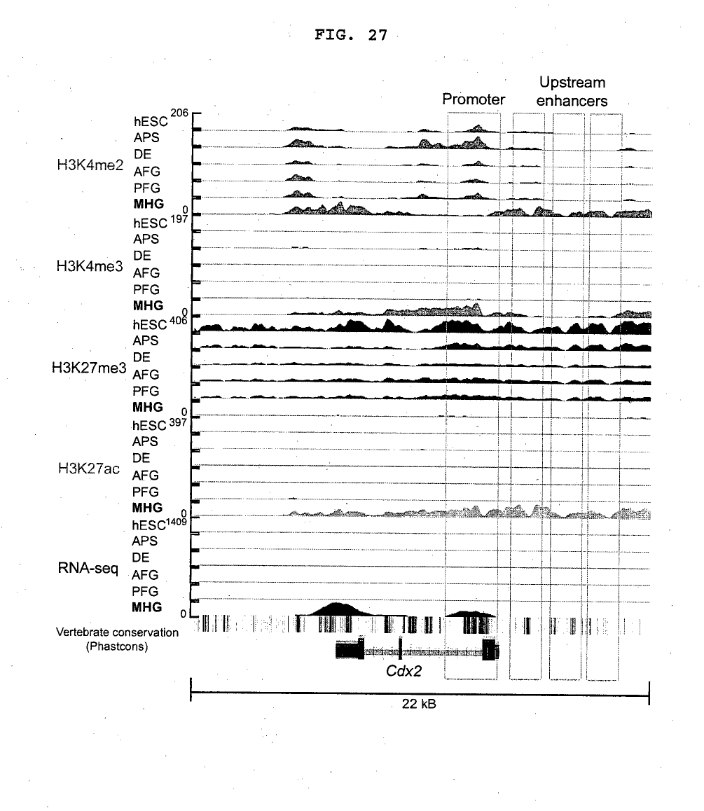

Active enhancers were assigned from aligned and input-normalized H3K27ac ChIP-seq data using DFilter (Kumar et al., 2013). By treating peak-calling from ChIP-seq data as a signal-detection problem, DFilter uses formally optimal solutions from signal-processing theory to identify ChIP-seq peaks of variable width. Briefly, DFilter detects peaks in the ChIP-seq signal by attempting to maximize the receiver area characteristic-area under the curve (ROC-AUC) by employing a linear detection filter (the Hotelling observer) to maximize the ChIP-seq signal difference between “true” positive regions and noise regions. H3K27ac peaks were individually identified by DFilter in each of the six cell types (hESC, APS, DE, AFG, PFG and MHG), using a kernel size of 6 kB and a zero-mean filter, and all peaks were required to have ≧15-fold H3K27ac tags in at least one 100 bp bin than in the corresponding input library bin (control local tag density). Peaks mapping to chr random contigs, segmental duplications, satellite repeats and ribosomal RNA repeats were removed. Thereafter, peaks within 1 kB of any RefSeq TSS or UCSC Known Gene TSS were cropped to yield distal peaks. Overlapping distal H3K27ac peaks from each of the six cell types were then merged, yielding a union of all enhancers active in at least one of the lineages examined. The outcome of this active enhancer union was represented, after binary clustering, in FIG. 26.

To identify “cell type-specific active enhancers” (e.g., DE-specific active enhancers), an enhancer was required to have ≧4-fold more H3K27ac tags within the peak region in the given lineage (e.g., DE) versus undifferentiated hESC (thus identifying enhancers that gain significant amounts of H3K27ac upon differentiation). This cohort of 10,543 “DE-specific active enhancers” was subsequently used for gene-ontology and motif analyses.

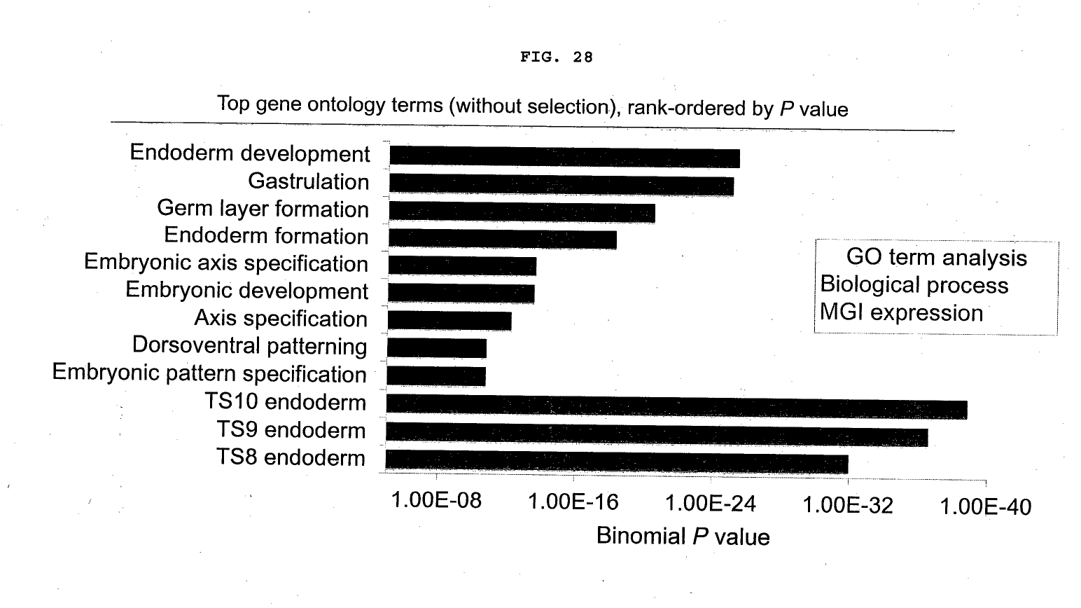

Gene ontology terms associated with endoderm-specific active enhancers were ascertained via GREAT (McLean et al., 2010): for each enhancer, the nearest gene within 100 kB was used (“basal plus extension”, eliminating elements 1 kB upstream or 2 kB downstream from the TSS). In FIG. 28, the most significantly-associated GO terms (Biological Process and MGI Expression) are depicted, rank ordered by P value as displayed on the online GREAT portal (http://bejerano.stanford.edu/great/public/html/) without prior preselection or prefiltering of any terms.

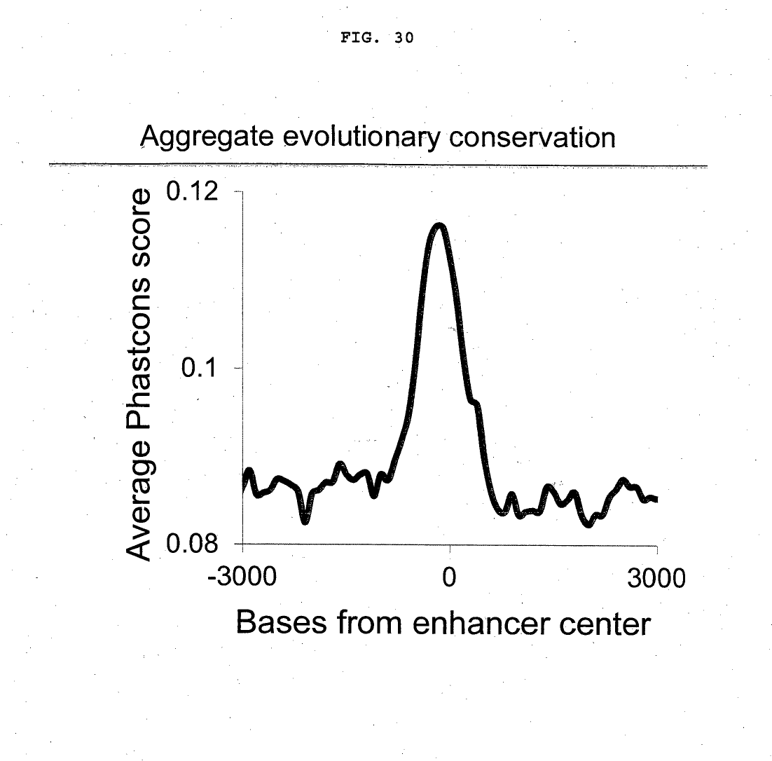

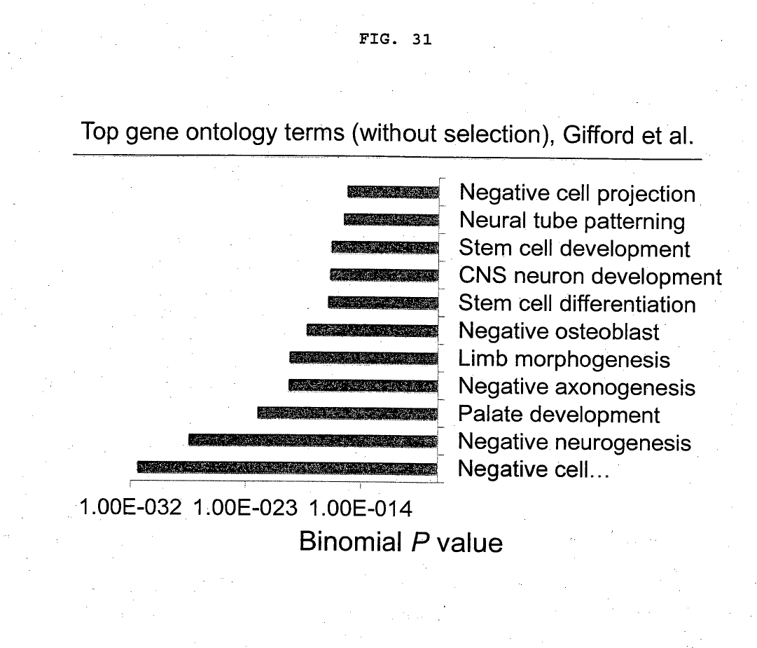

Average evolutionary conservation of endoderm enhancers was assessed using the Conservation Plot function of Cistrome (http://cistrome.org/ap/) within a ±3 kB window surrounding the enhancer center as displayed in FIG. 30.

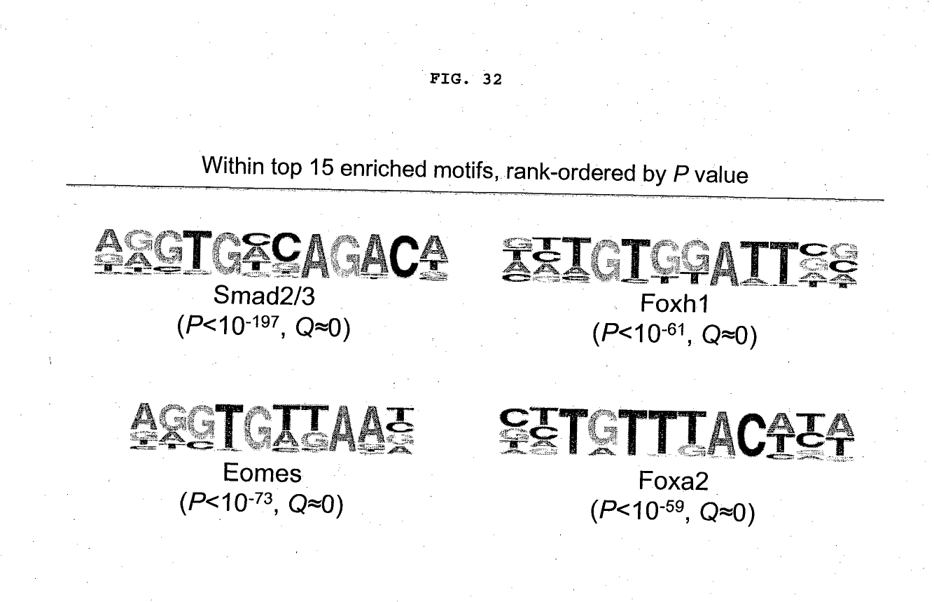

Transcription-factor motifs enriched in DE-specific enhancers were determined using HOMER (Heinz et al., 2010) (http://biowhat.ucsd.edu/homer/chipseq/) and representative transcription-factor motifs within the top 30 hits were displayed in FIG. 32.

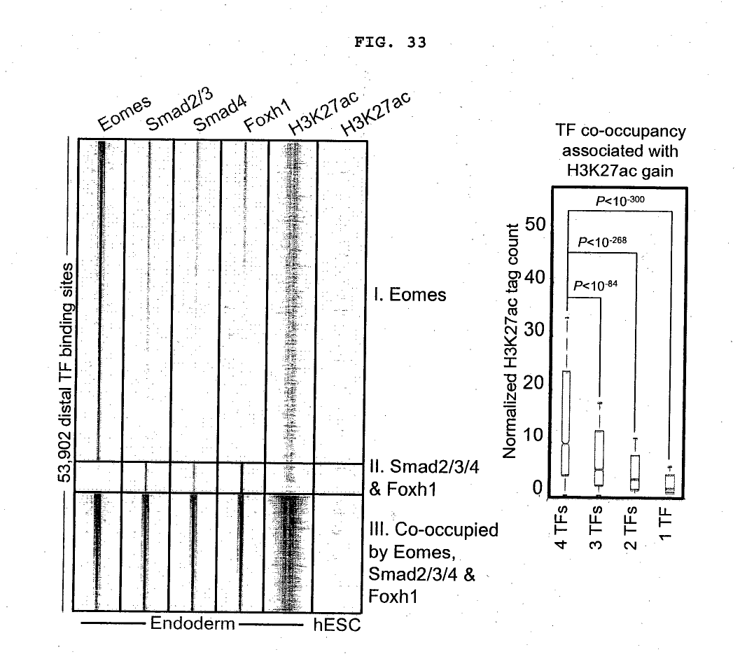

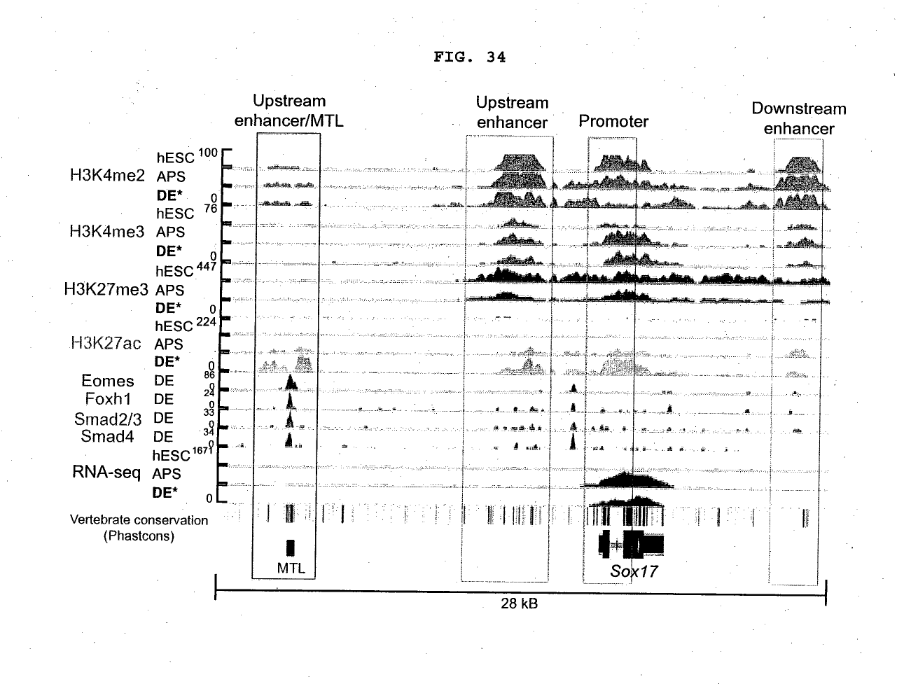

To understand how endodermal TFs converge on active DE enhancers, Eomes, Smad2/3, Smad4 and Foxh1 ChIP-seq data in DE (Kim et al., 2011; Teo et al., 2011) was downloaded from GEO (GSE26097 and GSE29422, respectively), aligned and input-normalized as described above and finally peaks were called using HOMER. The union of all DE TF ChIP-seq peaks was created, overlapping peaks were merged and all peaks within 1 kB of a RefSeq were eliminated to yield all 53,902 distal DE TF-binding sites. Using HOMER, binned tag counts surrounding each DE TF-binding site were extracted and k-means clustering was applied to identify three predominant classes of binding events: (i) Eomes-bound-alone, (ii) Smad2/3/4-and-Foxh1-bound, and (iii) co-bound by Eomes, Smad2/3/4 and Foxh1 and this was visualized in a spatial heatmap together with H3K27ac ChIP-seq data in DE and hESCs in FIG. 33.

### Comparison of SR1-Induced and Previous Endoderm Enhancer Signatures

ChIP-seq data of HUES64-derived DE populations differentiated by Activin A, Wnt3a and 0.5% FBS treatment for 4 days has been previously reported (Gifford et al., 2013) and H3K27ac ChIP-seq data for undifferentiated HUES64 and HUES64-derived Cxcr4+DE was downloaded (http://www.ncbi.nlm.nih.gov/geo/roadmap/epigenomics/?view=mat rix). Thereafter, HUES64 ChIP-seq data was processed identically as described above for SR1 ChIP-seq data: H3K27ac reads were aligned to hg19 and input-normalized to respective control libraries. To identify active enhancers enriched in HUES64-derived DE, H3K27ac peaks were assigned by DFilter (Kumar et al., 2013) and fold-change in H3K2ac tag counts in DE versus undifferentiated HUES64 was calculated. The top 10,000 DE-enriched enhancers (with highest H3K27ac fold-changes in DE vs. undifferentiated HUES64) were called: to provide an unbiased comparison, the top 10,000 DE-enriched enhancers from the SR1 DE dataset was called by comparing SR1 DE H3K27ac tag count fold-change against undifferentiated HUES64. Subsequently, the top 10,000 DE-enriched active enhancers drawn from the SR1 dataset or the Gifford et al. dataset were extracted and enriched GO terms were associated side-by-side using GREAT (McLean et al., 2010) with the following parameters: single nearest gene, 1,000,000 bp max extension and curated regulatory domains included. The results of this side-by-side DE enhancer comparison are presented in FIG. 31.

Identifying Pre-Enhancer Chromatin States in hESC

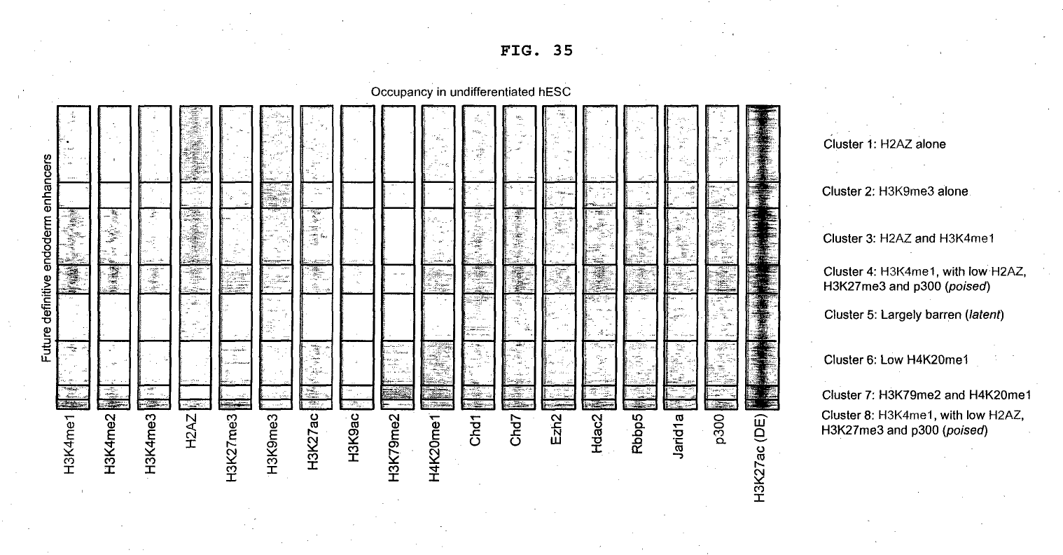

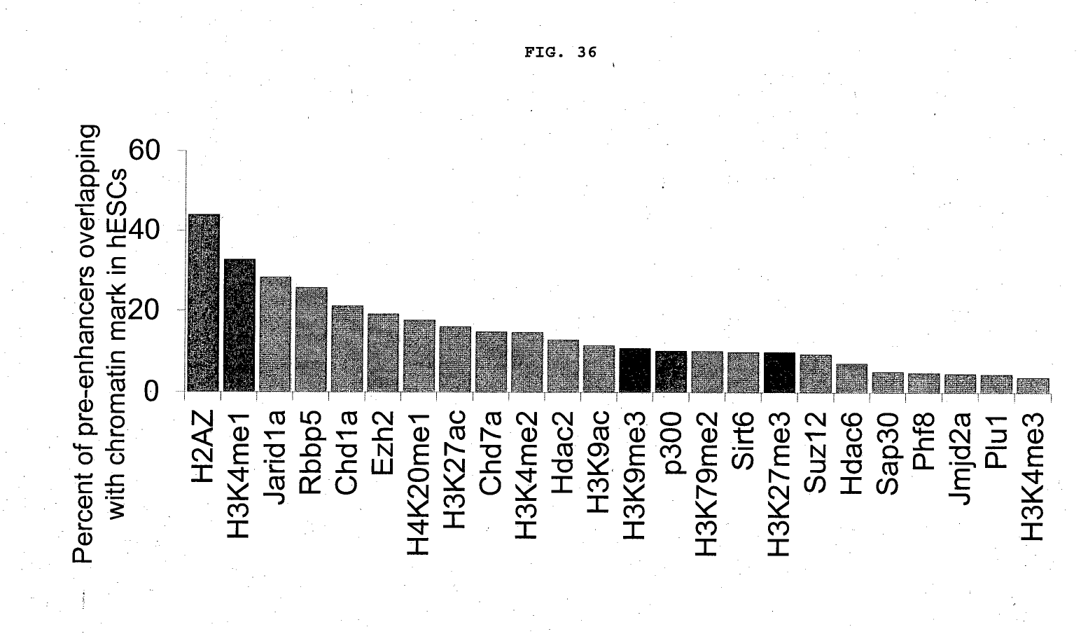

To ascertain how DE enhancers are marked in undifferentiated hESC prior to differentiation, we first pre-filtered the above list of 10,543 DE-specific enhancers to fully discard any peaks ±3 kB of a TSS in order to minimize bleedthrough of promoter signals. We downloaded ChIP-seq data for >24 marks: 10 histone modifications (H3K4me1, H3K4me2, H3K4me3, H3K9me3, H3K36me3, H3K79me2, H4K20me1, H3K9ac, H3K27ac & H2AZ) (Ernst et al., 2011) and 14 chromatin regulators (Chd1, Chd7, Ezh2, Hdac2, Hdac6, Jarid1a, Jmjd2a, p300, Phf8, Plu1, Rbbp5, Sap30, Sirt6, Suz12) (Ram et al., 2011) from GEO (GSE29611) or the UCSC Genome Browser download portal (http://hgdownload.cse.ucsc.edu/goldenPath/hg19/encodeDCC/wgEn codeBroadHistone), respectively. This was done in order to comprehensively assess occupancy of DE enhancers in hESC by virtually most known histone modifications and chromatin regulators, with the goal of systematically identifying all possible “pre-enhancer” states. In order to identify coherent patterns of “pre-enhancer” chromatin states we prepared ChIP-seq data for clustering: to cluster multiple histone modification and chromatin regulator ChIP-seq signals at given enhancers, first each ChIP-seq signal was decomposed into the form of tag-count in 200 bp bins across the enhancer region. The binned tag-count signal was normalized by the mean tag count in the entire library. The log of the normalized tag-count signal was used to make a spatial heatmap (FIG. 35) and for further clustering. For each ChIP-seq library, the maximum binned tag-count within 1 kB of the enhancer center was represented in a column of a 2-dimensional (n×k) matrix, where n is the number of DE enhancers analyzed and k is the total number of ChIP-seq libraries examined. This 2D matrix was used for k-means clustering (Matlab) to learn pre-enhancer classes. After learning pre-enhancer classes, one 2D matrix (n×2w) was made for each ChIP-seq library, taking signals in w bins around each enhancer as a row of the matrix. Then for each ChIP-seq library the calculated 2D matrix (n×2w) was plotted using the imagesc function (Matlab). To assess the relative prevalence of histone modification and chromatin regulator “pre-marking” of DE pre-enhancers in hESC, coverage of all DE pre-enhancers with histone modification and chromatin regulator ENCODE peak-calls (http://hgdownload.cse.ucsc.edu/goldenPath/hg19/encodeDCC/wgEn codeBroadHistone) was ascertained (FIG. 36). For the sake of easy visual representation, only histone modifications or chromatin regulators that marked more than ˜5% of DE enhancers were represented in FIG. 35-36. To identify mesodermal pre-enhancer classes in hESCs, similar procedures were employed as those used to assess endoderm pre-enhancers, with the exception that the list of mesoderm active enhancers was abstracted from previous H3K27ac ChIP-seq profiling of CD56/NCAM1+ hESC-derived mesoderm populations (Gifford et al., 2013).

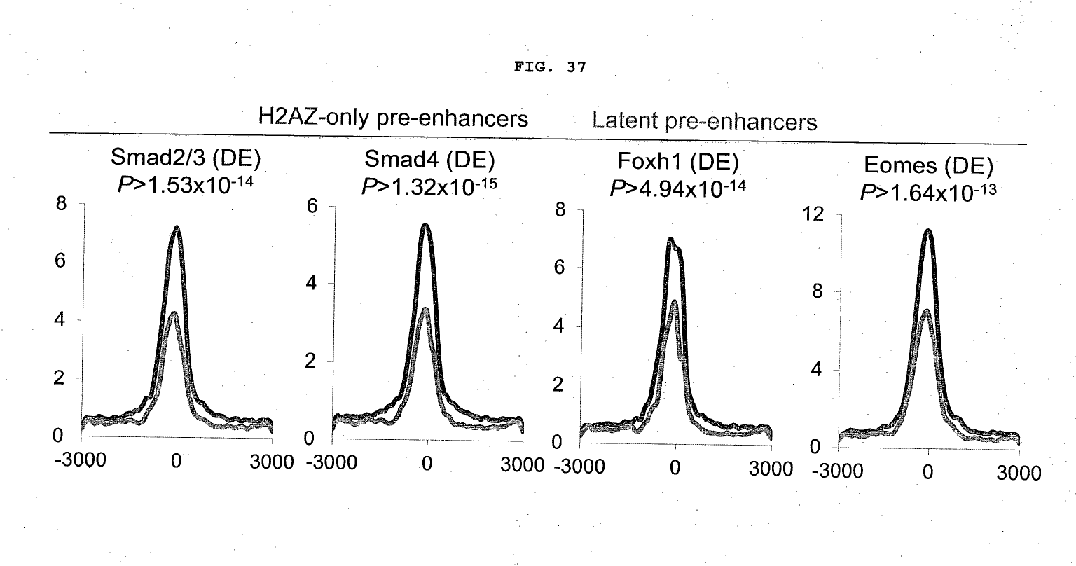

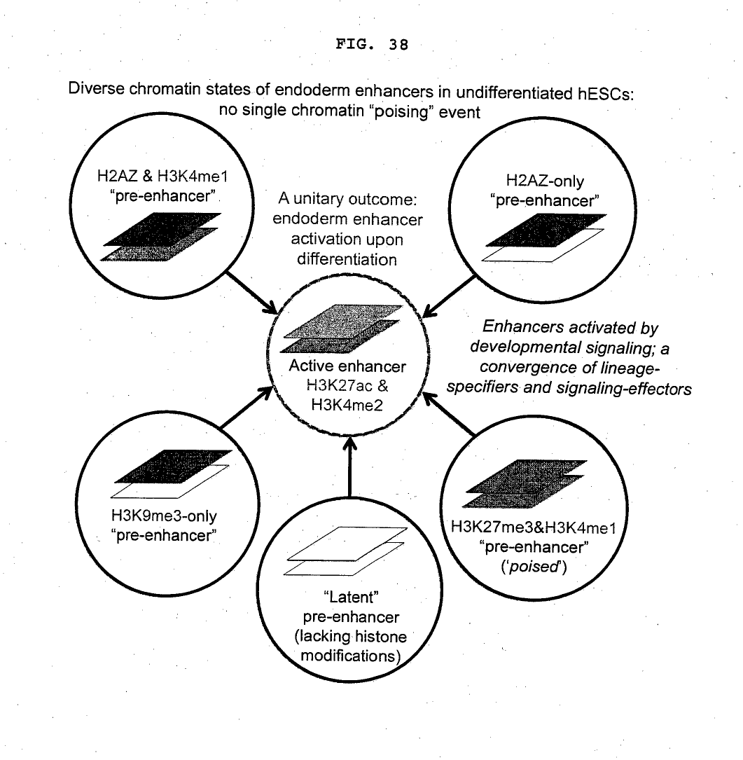

To globally assess occupancy of different pre-enhancer classes by DE TFs upon DE differentiation, average Eomes, Smad2/3, Smad4 and Foxh1 ChIP-seq signals in DE were plotted across all class 1 pre-enhancers (H2AZ-only) and all class 5 pre-enhancers (largely latent) with a 6 kB window size (FIG. 37).

### ChIP-Seq, RNA-Seq, and Microarray Data Deposition

Raw ChIP-seq, RNA-seq, and microarray data for endoderm differentiation (summarized in FIG. 20) have been deposited online at http://collaborations.gis.a-star.edu.sg/˜cmb6/kumarv1/endoderm/under username ‘review123’ and password ‘review’. Raw data will be uploaded to a public online repository upon acceptance.

### Experimental Results

### A Dynamic Switch in BMP and Wnt Signaling Induces Primitive Streak and Subsequently Suppresses Definitive Endoderm Emergence

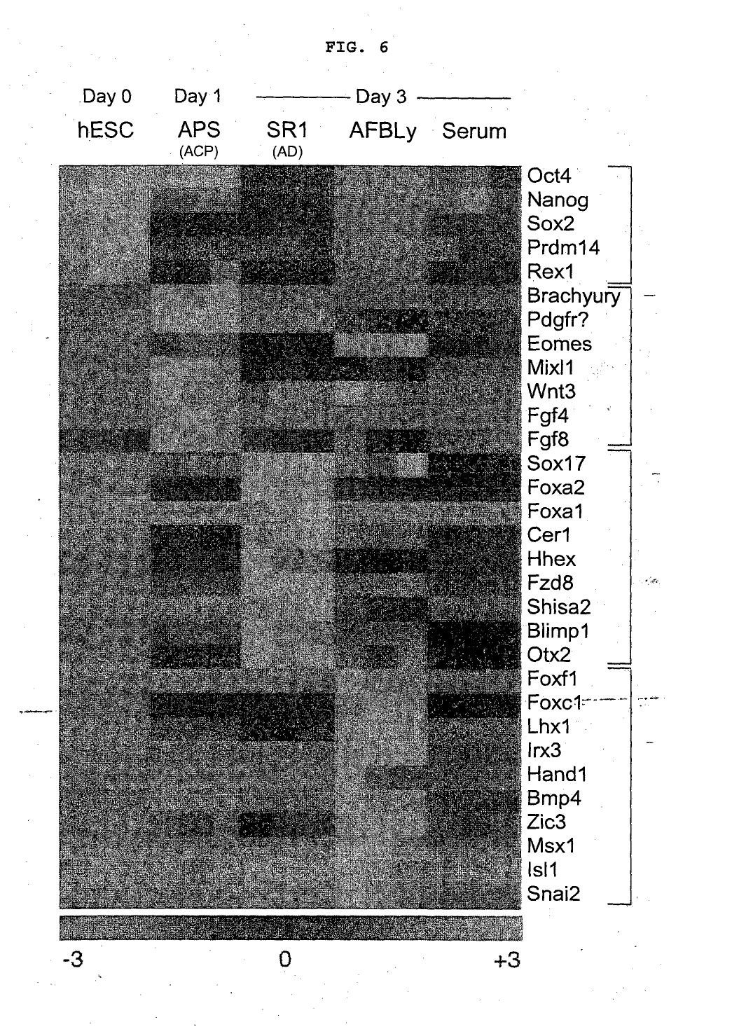

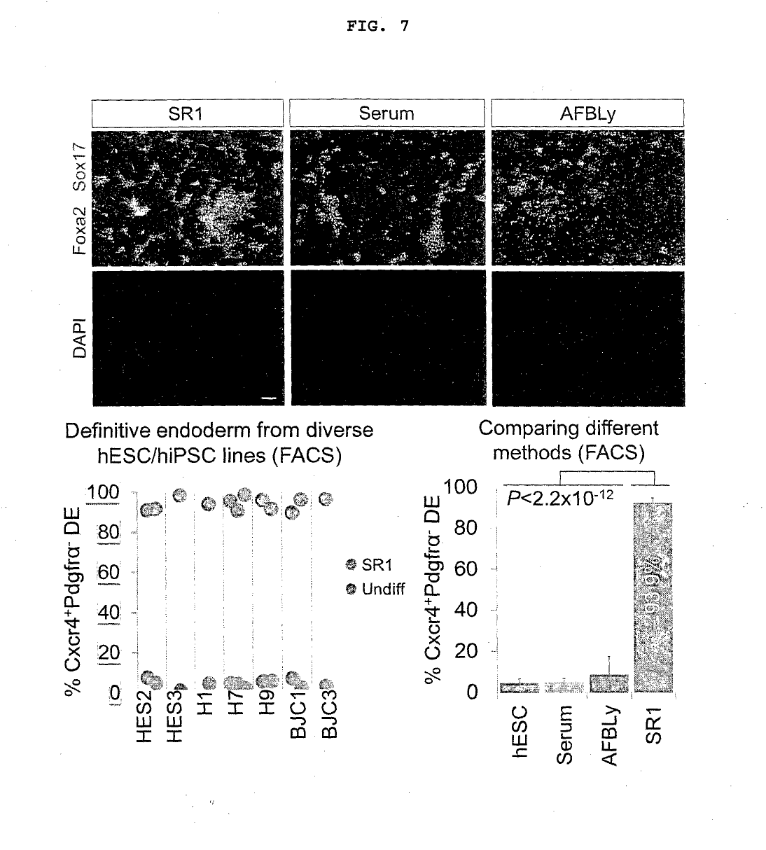

This was preceded by findings that Activin, in conjunction with FGF, BMP and a PI3K inhibitor (“AFBLy”) (Touboul et al., 2010) or together with animal serum (D'Amour et al., 2005), specified DE from hESC. However these methods still yielded mixed lineage outcomes, evident during the differentiation of 5 hESC lines (FIG. 1, FIG. 6-7, FIG. 39-61). For example, AFBLy (Touboul et al., 2010) concurrently generated mesoderm, upregulating skeletal, vascular and cardiac genes (P<10⁻⁸; FIG. 1, FIG. 39-42), whilst Activin and serum treatment (D'Amour et al., 2005) yielded a proportion of undifferentiated cells (FIG. 6-8). Creation of impure early DE populations might explain the emergence of non-endoderm lineages after downstream differentiation (Kroon et al., 2008; Rezania et al., 2012).

Developmental signals were selectively perturbed (individually or in combination, >3,200 signaling conditions) at specific embryonic stages of hPSC differentiation in serum-free conditions and assessed resultant lineage outcomes by qPCR (yielding >16,000 datapoints, FIG. 39-63). These signaling perturbations revealed elements of the signaling logic underlying DE induction (FIG. 1-23).

In vivo, DE arises from the primitive streak (PS, ˜E6.5) (Levak-\check{S}vajger and \check{S}vajger, 1974). The anteriormost PS (APS) generates DE (˜E7.0-E7.5) whereas posterior PS (PPS) forms mesoderm (Lawson et al., 1991; Tam and Beddington, 1987).

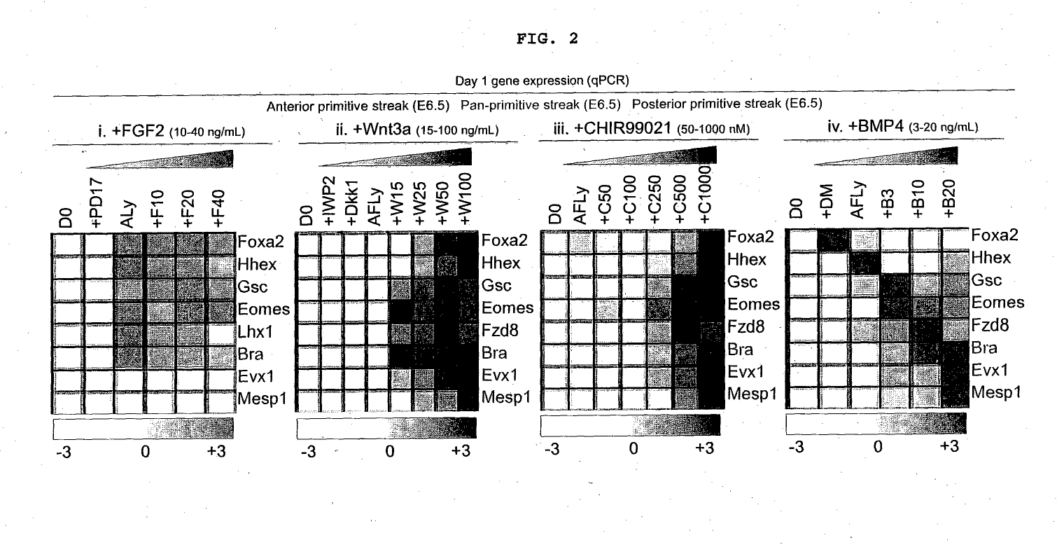

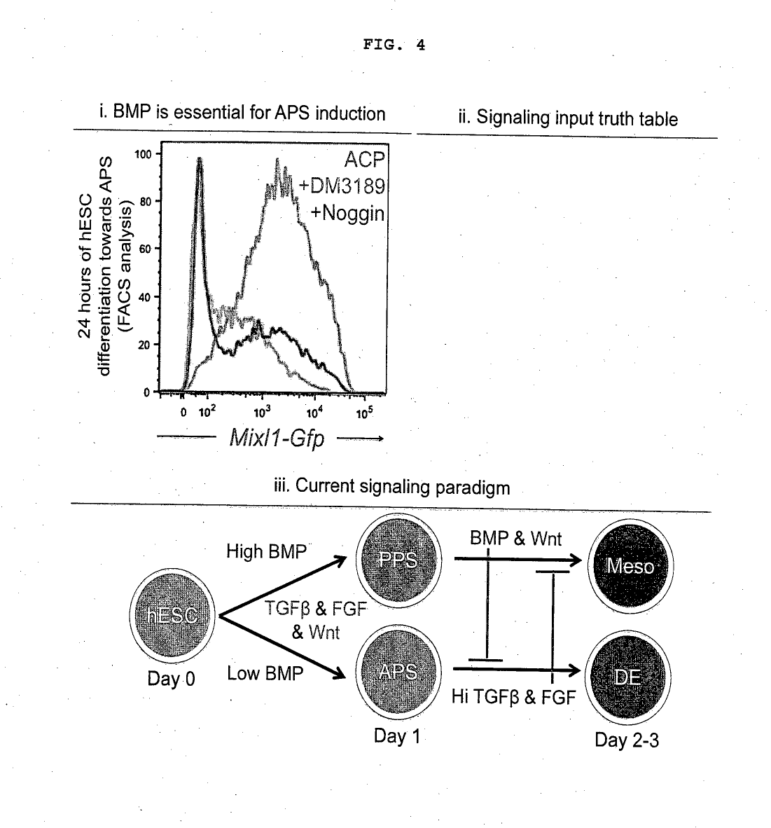

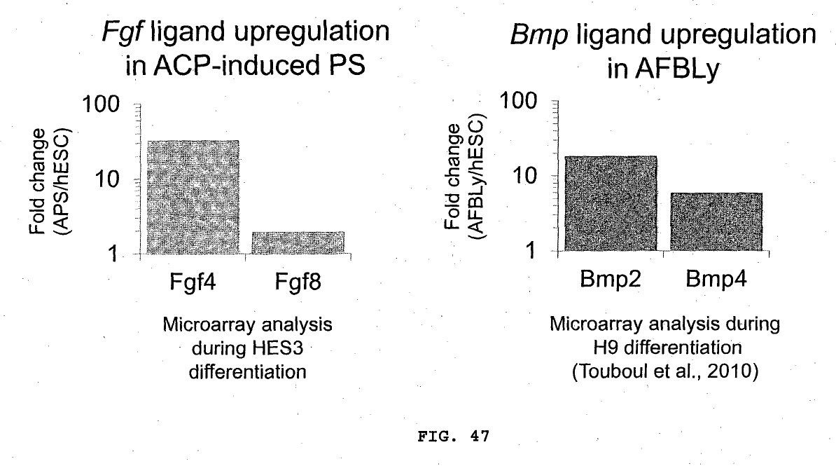

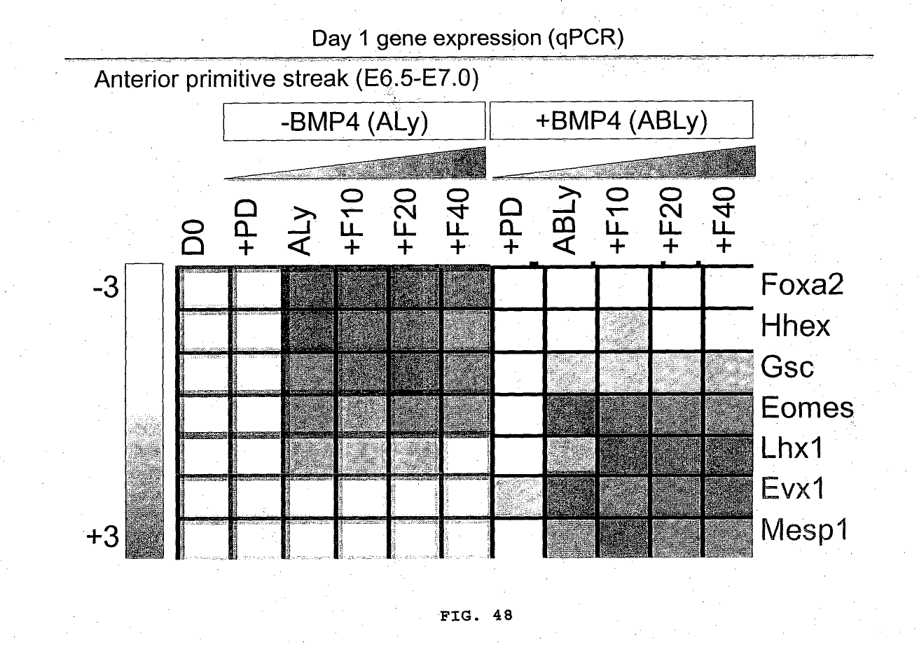

Both APS and PPS were combinatorially induced by BMP, FGF and Wnt on day 1 of hESC differentiation. These signals have been individually implicated in PS induction (Bernardo et al., 2011; Blauwkamp et al., 2012; Gadue et al., 2006) but their roles in PS patterning have not been dissected in detail. If either BMP, FGF or Wnt was inhibited, both APS and PPS formation failed (FIG. 2), corroborating the lack of PS in BMP and Wnt pathway knockout mice (Beppu et al., 2000; Liu et al., 1999; Mishina et al., 1995). FGF signaling was equally permissive for both APS and PPS emergence and endogenous FGF was sufficient to drive either outcome (FIG. 2i, FIG. 47-49). However, exogenous Wnt (either Wnt3a or GSK3 inhibition [CHIR]) was necessary to maximize PS induction and Wnt broadly promoted both APS and PPS (FIG. 2ii-iii). Limited PS formation could occur without exogenous Wnt but was dependent on endogenous Wnt (FIG. 2ii). BMP levels arbitrated between APS and PPS: lower (endogenous) BMP levels elicited APS, whereas higher BMP yielded PPS (FIG. 2iv, FIG. 48). However, the absolute necessity of BMP for MIXL1-GFP⁺ APS induction (FIG. 4i, P<0.025) was unexpected as BMP was typically associated with mesoderm formation (Bernardo et al., 2011). Therefore, FGF, Wnt and low BMP were essential for APS specification.

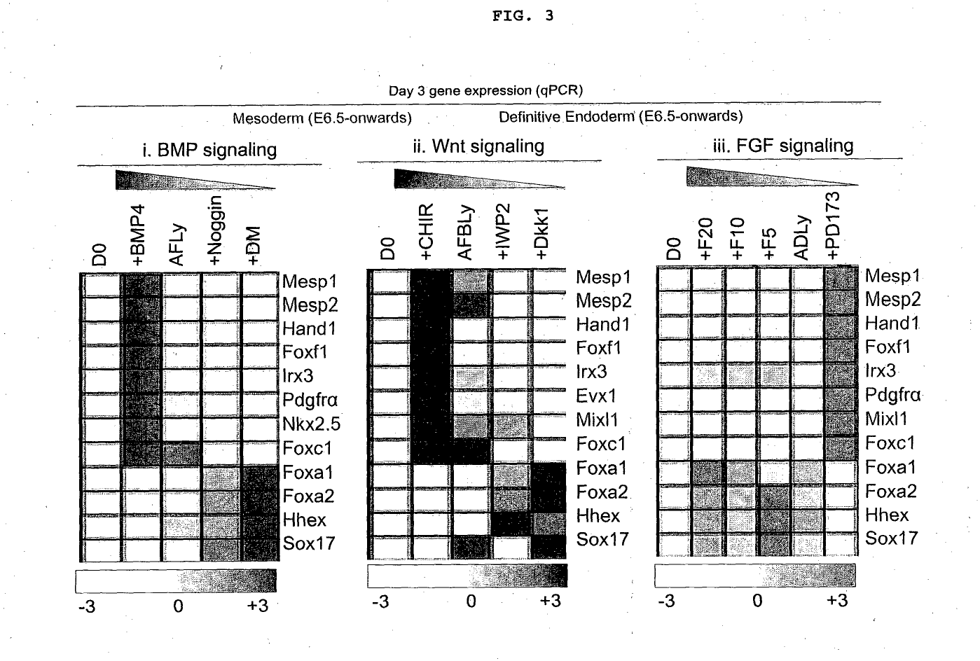

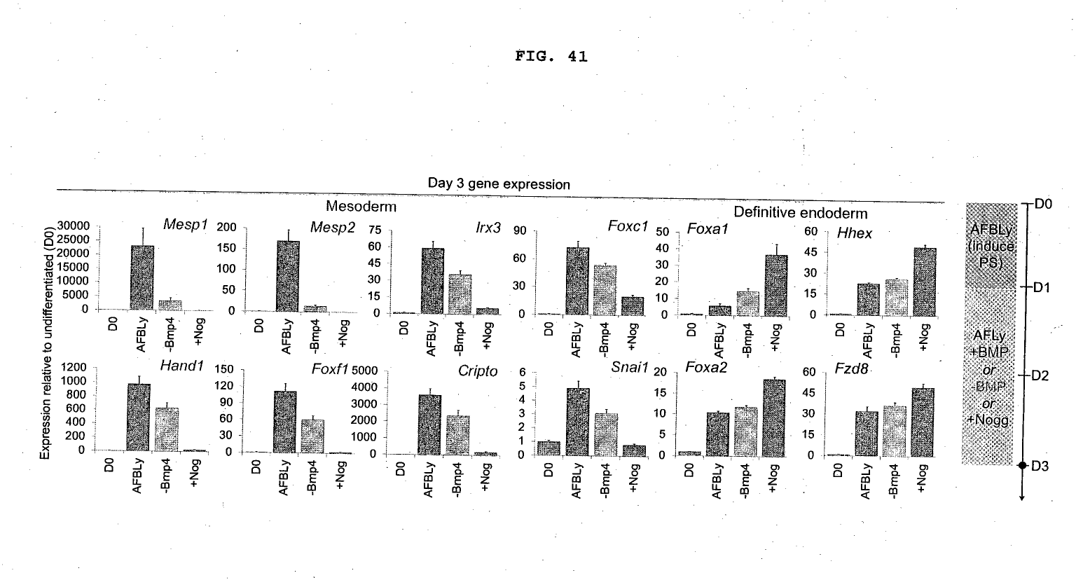

To further differentiate APS towards DE, prior studies used similar factors to induce both lineages over 3-5 days (Nostro et al., 2011; Touboul et al., 2010). Instead APS and DE were sequentially driven by diametrically opposite signals within 24 hours of differentiation. BMP and Wnt initially specified APS from hESC on day 1, but 24 hours later, BMP and Wnt induced mesoderm and reciprocally repressed DE formation from PS on days 2-3 of differentiation (FIG. 3i-ii). Interestingly, not only removing exogenous BMP but neutralizing endogenous BMP (using noggin or DM3189/LDN-193189) was necessary to eliminate mesoderm and to reciprocally divert PS differentiation unilaterally towards DE (FIG. 3i). This was evinced by ˜3000-fold downregulation of MESP1 and concurrent upregulation of SOX17, HHEX, FOXA1 and FOXA2 in 2 separate hESC lines (FIG. 41-43). Given that prolonged BMP and Wnt were known to induce mesoderm (Bernardo et al., 2011; Gadue et al., 2006; Gertow et al., 2013), the results altogether argue against prior sustained BMP treatment to induce DE from hESC (Cheng et al., 2012; Goldman et al., 2013; Nostro et al., 2011; Touboul et al., 2010), which we show abrogated DE and instead specified mesoderm. Timed BMP inhibition also improved DE induction from mESC, although which developmental step(s) BMP inhibition acted at remained unclear (Sherwood et al., 2011).

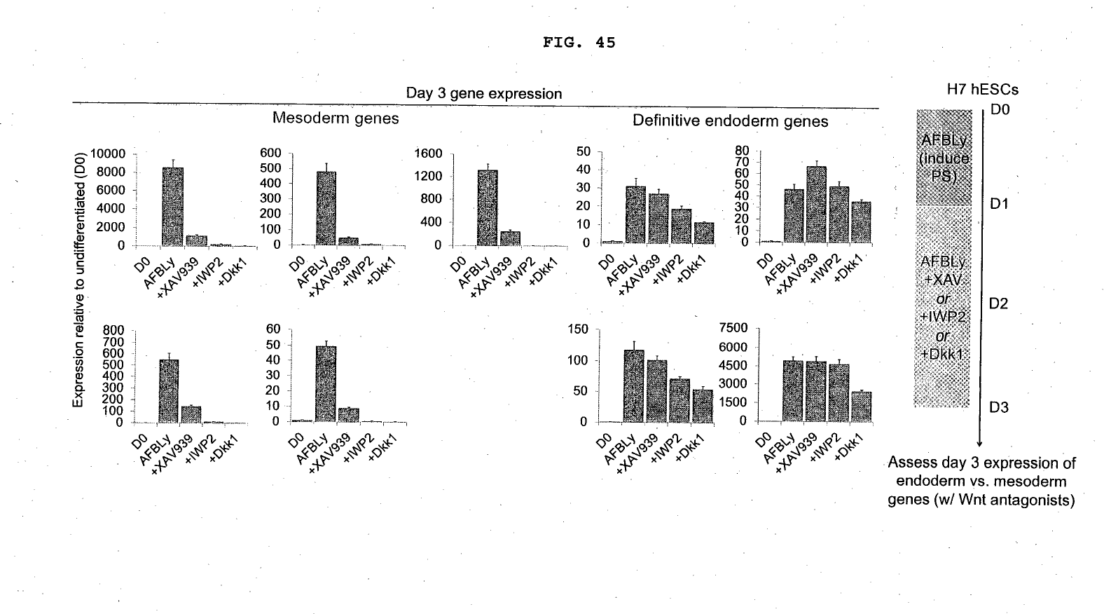

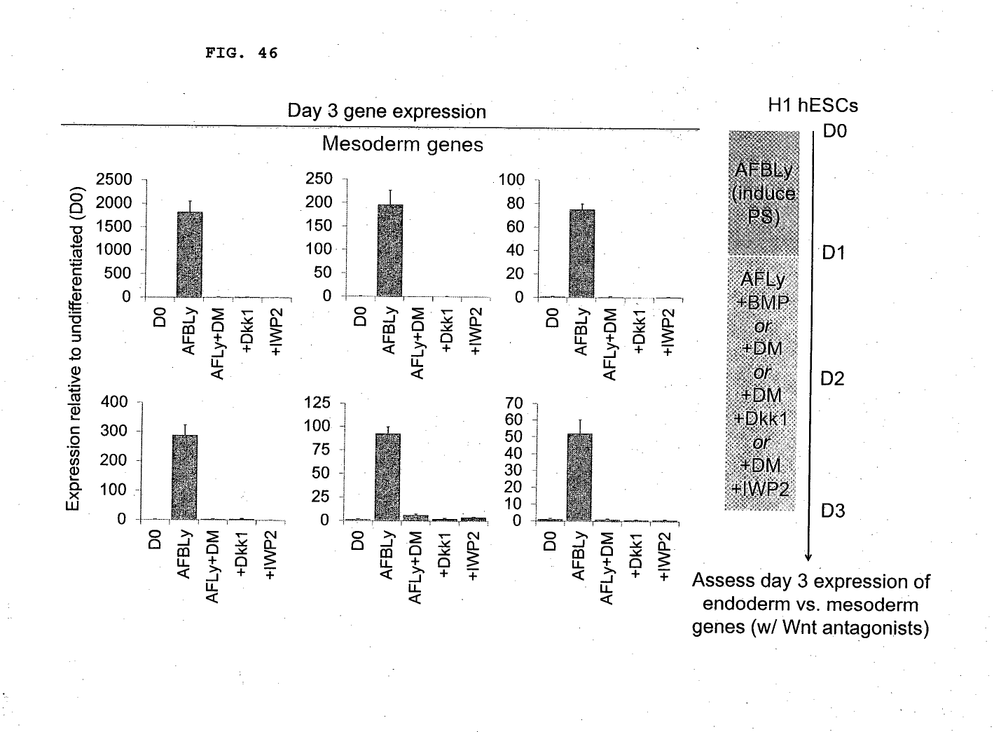

Similarly, endogenous Wnt/β-catenin signals directed PS towards mesoderm, such that inhibiting endogenous Wnt (using IWP2, Dkk1 or XAV939) on days 2-3 blocked mesoderm formation from 0.2 hESC lines (FIG. 3ii, FIG. 45-46). However, individually inhibiting either BMP or Wnt was sufficient to abolish mesoderm indicating that inhibiting both was redundant (FIG. 46). Thus, subsequently only BMP to derive DE from PS was inhibited. Finally, the results contrast with prolonged Wnt treatment to induce DE (Sumi et al., 2008), which we show instead specified mesoderm from PS and blocked DE. Altogether, BMP and Wnt induced mesoderm from PS and suppressed endoderm; therefore their inhibition ablated mesoderm and diverted differentiation towards DE.

While BMP and Wnt specified mesoderm, DE formation from PS was jointly driven by FGF (FIG. 3iii) in conjunction with TGFβ (Bernardo et al., 2011; D'Amour et al., 2005). If FGF was inhibited, mesoderm formation was re-enabled even in the absence of BMP (which is otherwise essential for mesoderm formation), showing FGF prevented illegitimate conversion of prospective DE to mesoderm. FGF is also essential for DE formation from mESC, yet paradoxically it was previously found that exogenous FGF was detrimental to DE induction (Hansson et al., 2009), which was not observed (FIG. 3iii).

In conclusion, these data uncovered a signaling cross-antagonism in which BMP and Wnt versus FGF and TGFβ respectively induced mesoderm versus endoderm from the PS and did so by cross-repressing the alternate fate (FIG. 4ii-iii). Furthermore, BMP and Wnt yielded dichotomous lineage outcomes depending on the developmental time of exposure—their effects became reversed within 24 hours (FIG. 4, FIG. 44).

Universal Generation of Highly-Purified DE from Diverse hPSC Lines Through Sequential APS Formation and Mesoderm Suppression

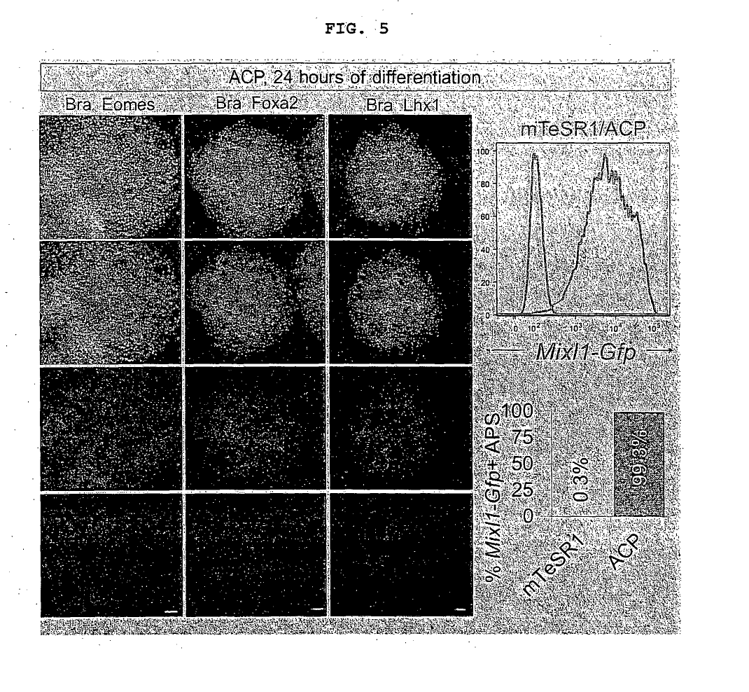

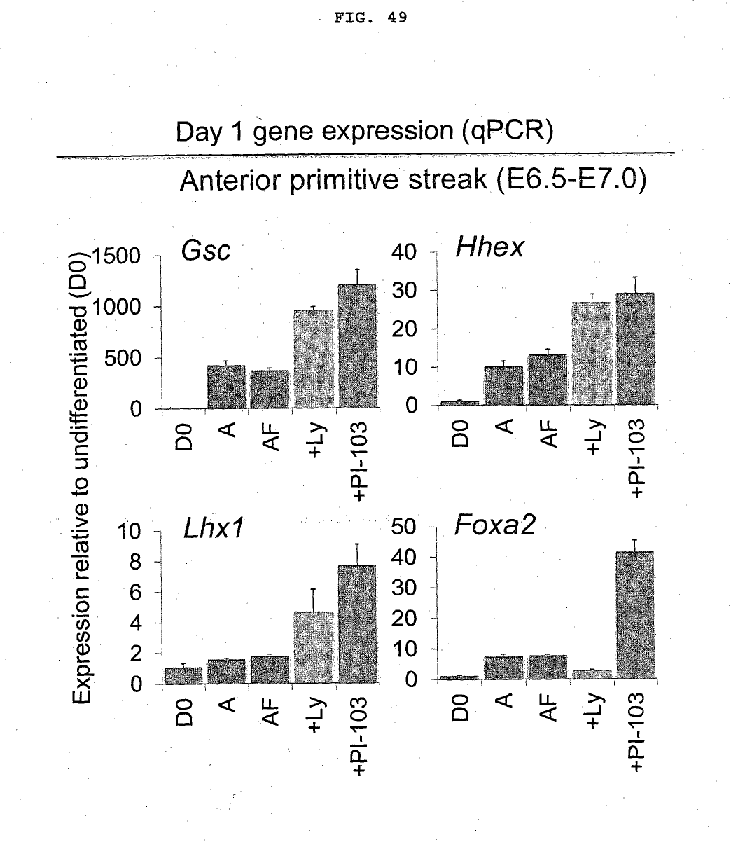

The above findings that APS and DE were sequentially specified by opposing signals, together with the necessity of BMP inhibition to eliminate mesoderm from the PS, motivated a serum-free monolayer approach (“SR1”) for DE induction. Firstly hPSC were differentiated to APS in 24 hours (FIG. 5) while excluding ectoderm by combining high Activin/TGFβ with CHIR (emulating Wnt/(3-catenin signaling) and PI3K/mTOR inhibition (FIG. 49-51), abbreviated “ACP”. This yielded a 99.3±0.1% MIXL1-GFP⁺ PS population (Davis et al., 2008) in which pan-PS TF BRACHYURY was coexpressed with APS-specific TFs EOMES, FOXA2 and LHX1 (FIG. 5, FIG. 54). 24 hours later, CHIR was withdrawn and APS was subsequently differentiated into DE by high Activin concomitant with BMP blockade (DM3189) to exclude mesoderm. Exogenous FGF was superfluous as endogenous FGF sufficed (FIG. 3iii, FIG. 47).

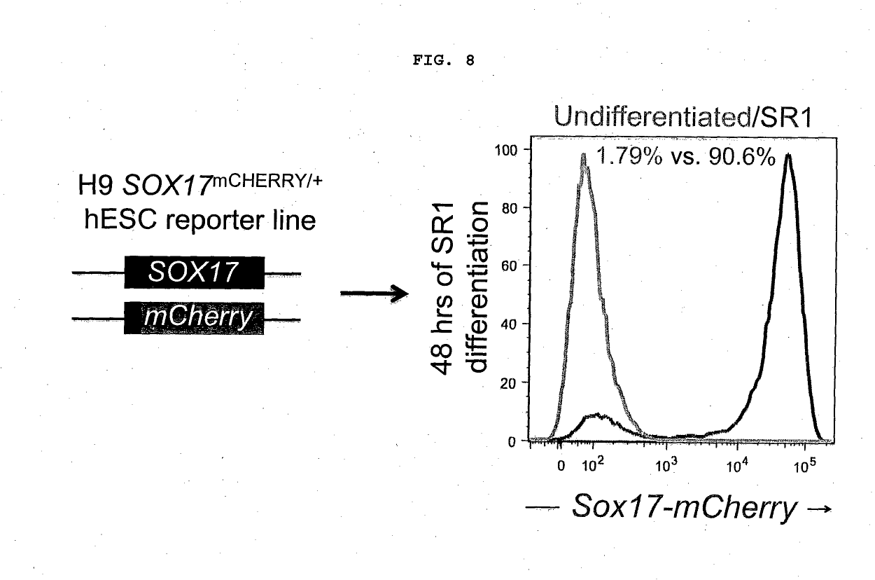

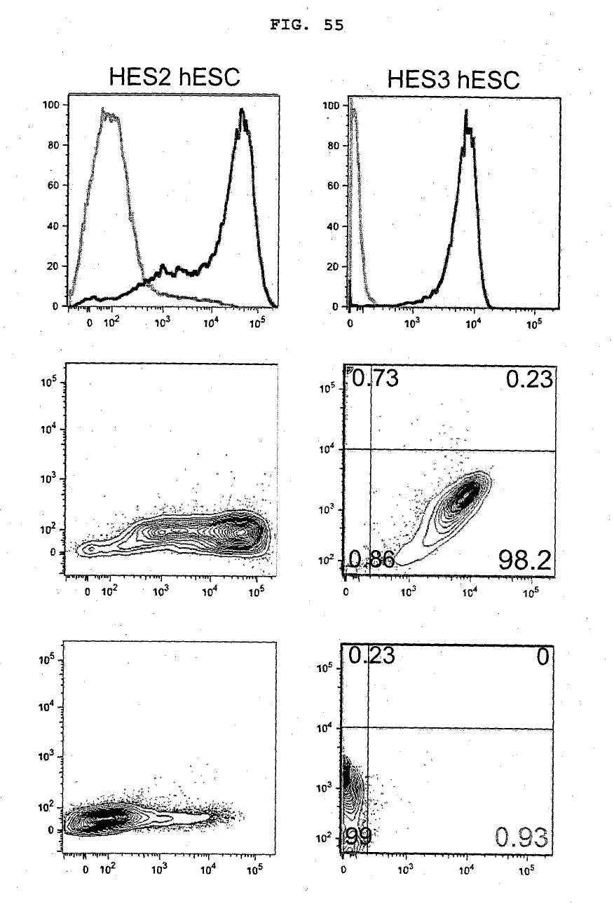

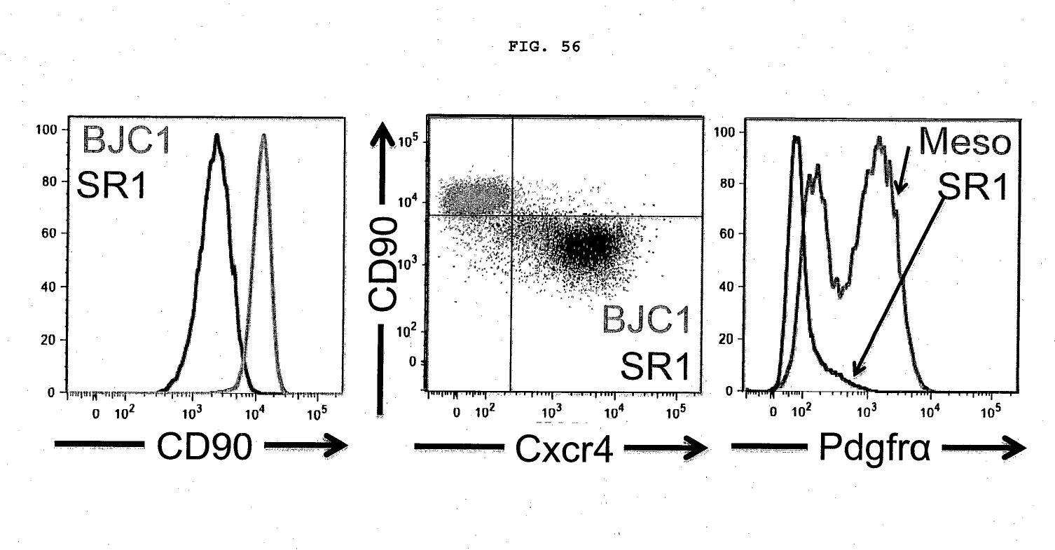

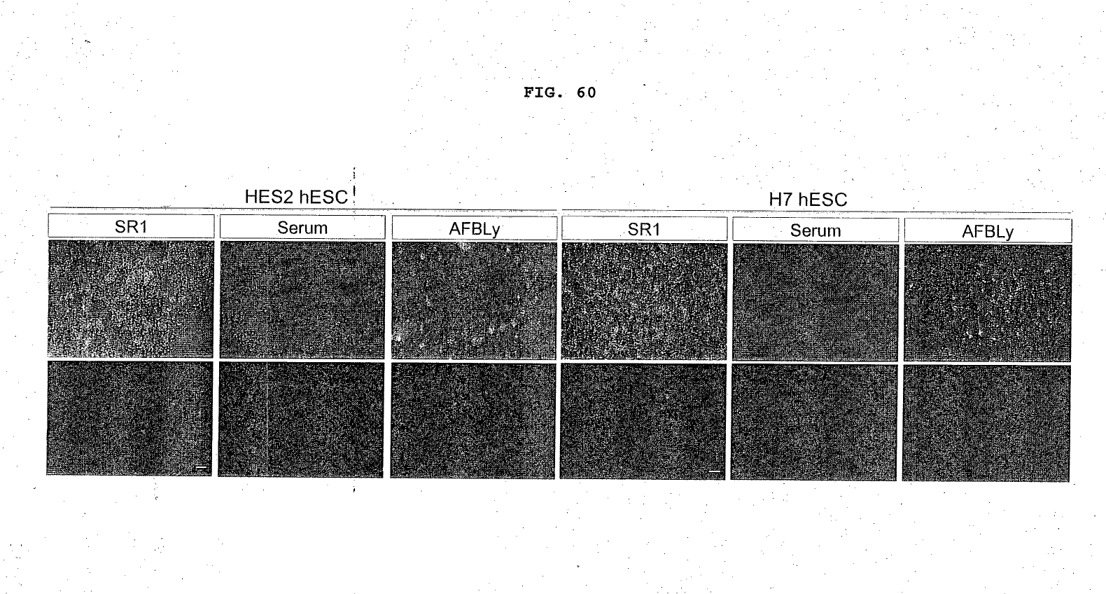

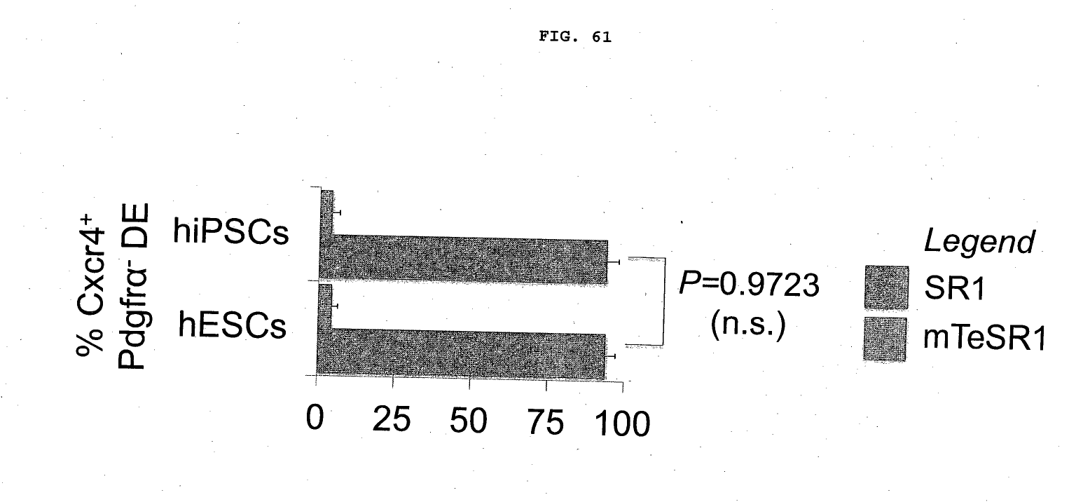

Sequential APS formation followed by DE induction universally yielded a 93.9±3.1% CXCR4⁺ PDGFRα⁻ DE population from 7 diverse hESC (H1, H7, H9, HES2 and HES3) and hiPSC (BJC1 and BJC3) lines within 3 days of differentiation (FIG. 6-9, FIG. 55), overcoming line-to-line induction variability. SR1 elicited broad FOXA2 and SOX17 coexpression (FIG. 7, FIG. 60) and downregulated hPSC marker CD90 (FIG. 56). hESC (94.0±3.1%) and hiPSC (93.9±3.9%) did not significantly differ in DE induction efficiencies (P>0.97, FIG. 61). A SOX17-mCHERRY knockin hESC reporter line was further exploited (LA, ESN, AGE, EGS, unpublished) to quantify differentiation efficiencies and found SR1 induced a >90% SOX17-mCHERRY⁺ DE population. (FIG. 8).

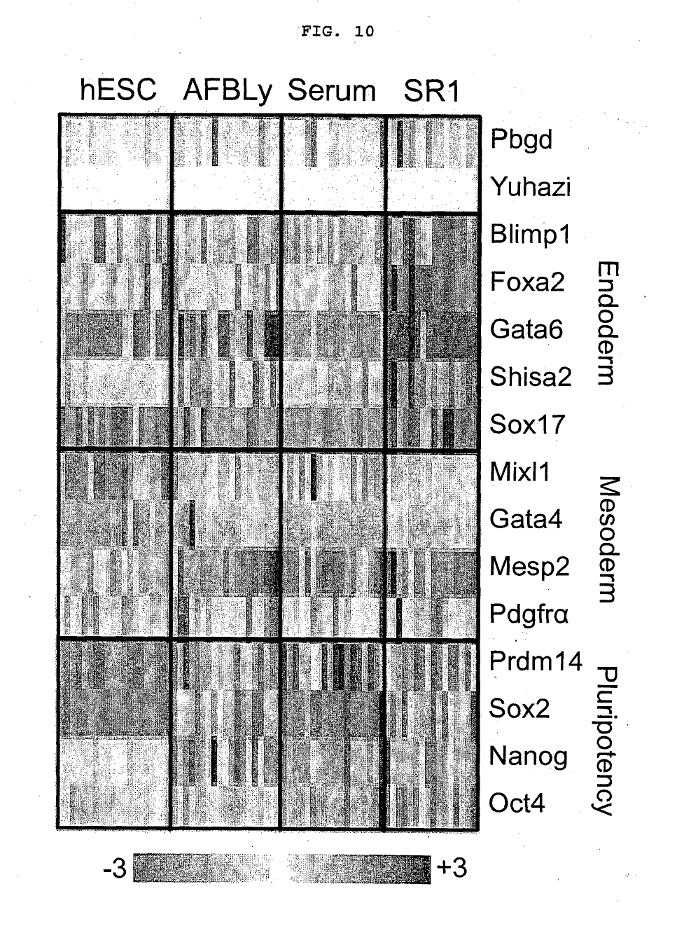

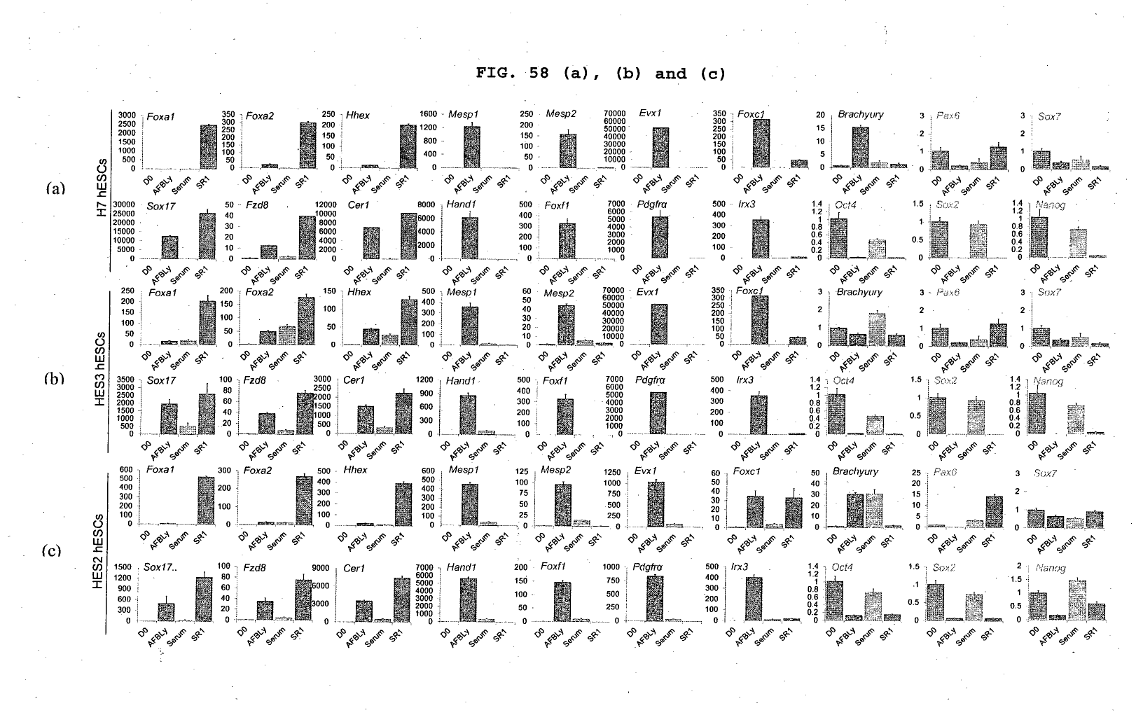

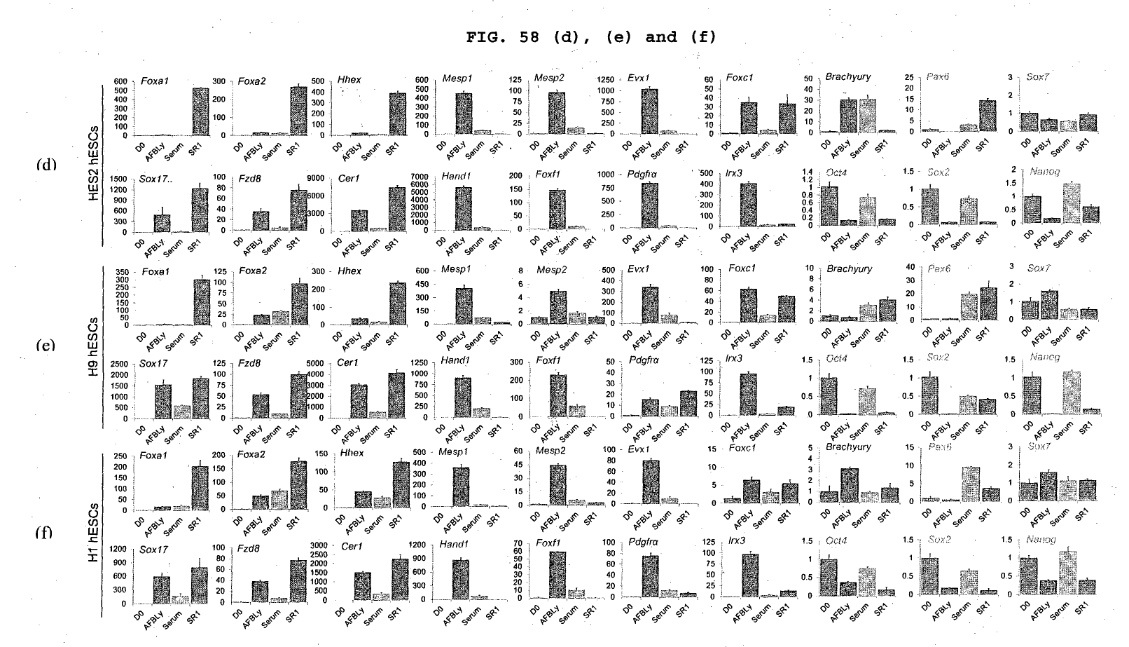

DE induction by SR1 was directly compared against two prevailing protocols, AFBLy (Touboul et al., 2010) or Activin and serum treatment (D'Amour et al., 2005) across 5 diverse hESC lines and tracked resultant lineage outcomes (FIG. 58 a-f). SR1 differentiation unilaterally yielded DE (SOX17, FOXA1, FOXA2, CER1, FZD8) from all 5 hESC lines with minimal mesoderm, extraembryonic endoderm or neuroectoderm (FIG. 6, FIG. 58 a-f). In contrast, the other DE protocols produced mixed lineage outcomes: AFBLy upregulated mesoderm TFs (FOXF1, HAND1, MSX1, ISL1) whereas pluripotency TF expression (OCT4, SOX2, NANOG) persisted after serum induction across all 5 lines (FIG. 6, FIG. 58 a-f). Accordingly, AFBLy and serum both produced lower SOX17⁺ FOXA2⁺ DE yields (FIG. 7, FIG. 60) and only modestly upregulated endoderm TFs (FIG. 6, FIG. 58 a-f). FACS quantification confirmed SR1 yielded purer DE than either AFBLy or serum treatment (P<2.2×10⁻¹²; FIG. 7, FIG. 58 a-f). At a clonal level, single-cell qPCR demonstrated endoderm TFs were robustly upregulated in the majority of SR1-induced cells: 20/20 cells were FOXA2⁺ (FIG. 10), wherein for each cell, gene expression values were normalized to Yuhazi (itself set as 0). Thereafter, anything lower than +6.5 was regarded FOXA2+ positive. In contrast, few cells in AFBLy-(1/20 cells) or serum-treated (2/20 cells) populations highly expressed FOXA2 (FIG. 10). Thus, even though all 3 differentiation protocols utilized high Activin, clearly Activin alone was insufficient to generate pure DE.

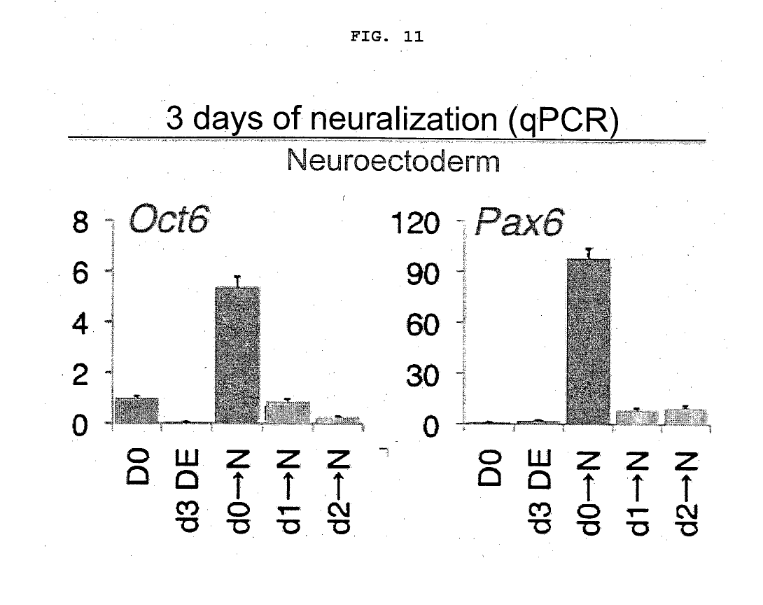

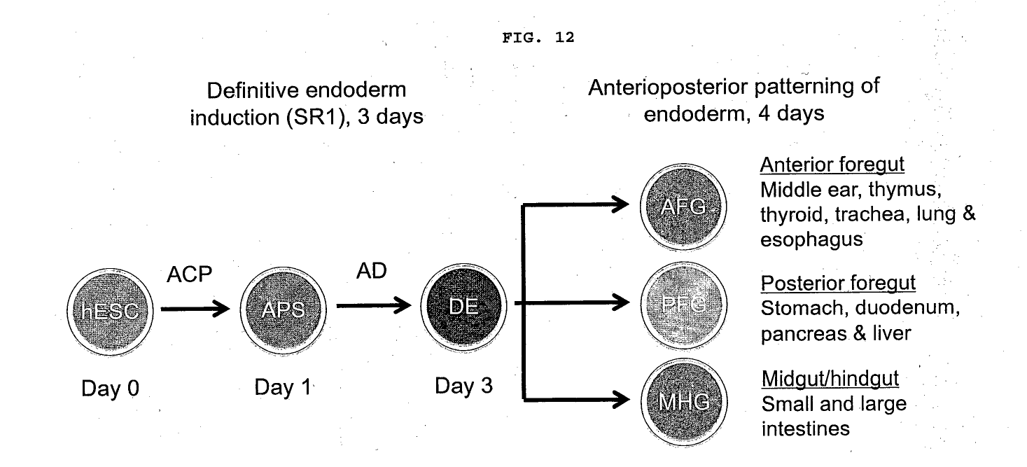

Finally, neural competence was relinquished within hours of SR1 induction (FIG. 11), showing mutually exclusive lineage potentials were lost upon APS/DE commitment.

Mutually Exclusive Anteroposterior Patterning of hESC-Derived DE into AFG, PFG and MHG Domains by BMP, FGF, RA, TGFβ and Wnt Signaling

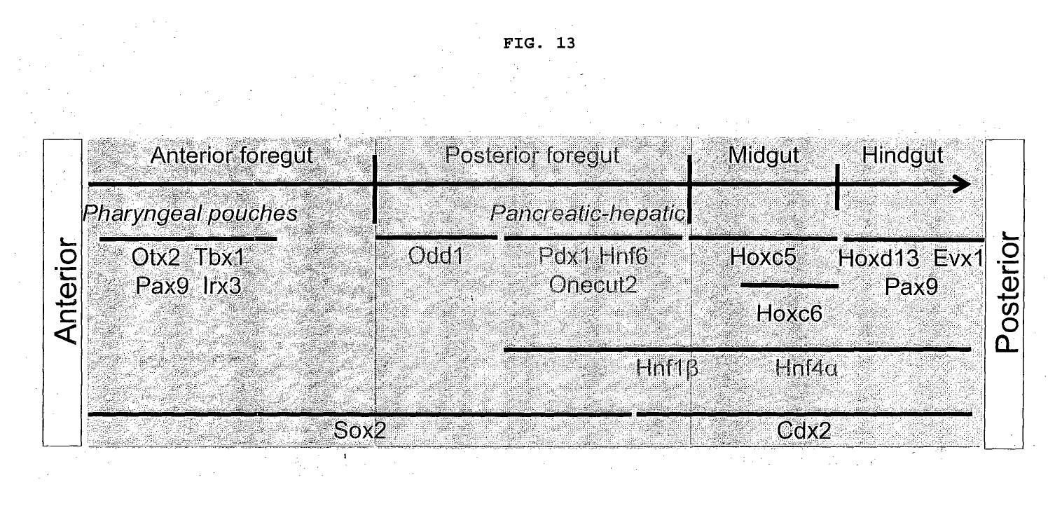

After its initial specification in vivo, DE is patterned along the anteroposterior axis into distinct domains which are the regional antecedents to endodermal organs (Zorn and Wells, 2009). The anterior foregut (AFG) gives rise to lungs and thyroid, the posterior foregut (PFG) to pancreas and liver and the midgut/hindgut (MHG) to small and large intestines (FIG. 12-13). Therefore, having induced mostly homogeneous DE from hPSC by day 3, we next attempted to anteroposteriorly pattern it into distinct AFG, PFG or MHG populations by 4 subsequent days of differentiation (FIG. 12), based on increasing knowledge of signals controlling DE patterning in vivo (Zorn and Wells, 2009) and in vitro (e.g., Green et al., 2011; Sherwood et al., 2011; Spence et al., 2011).

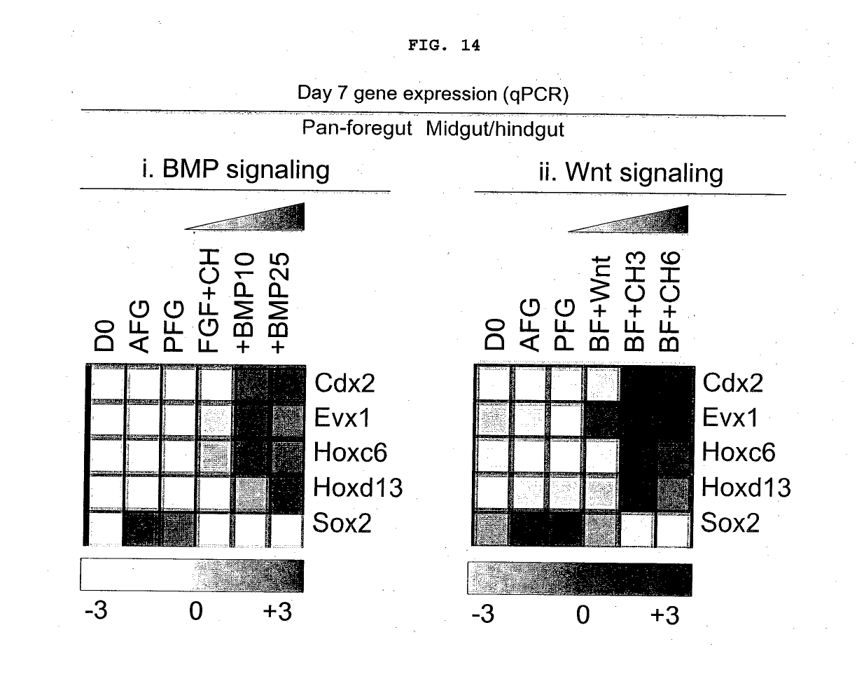

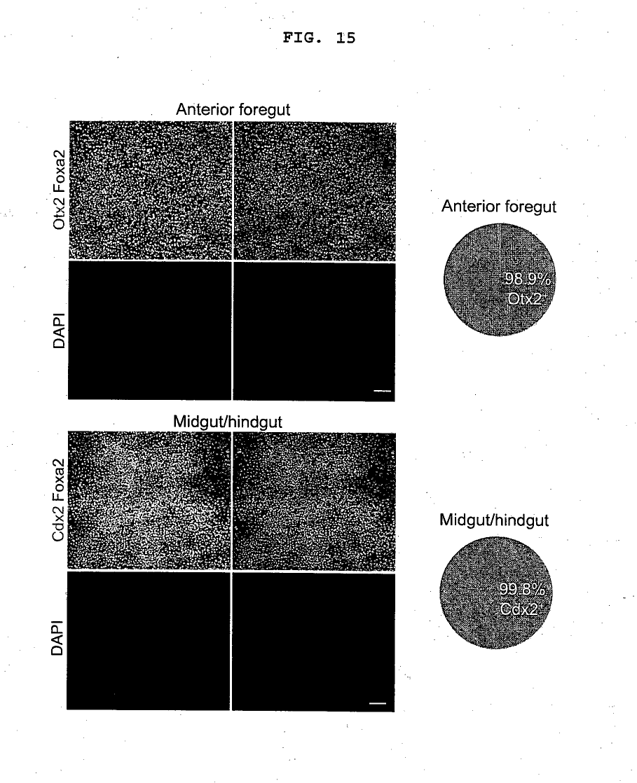

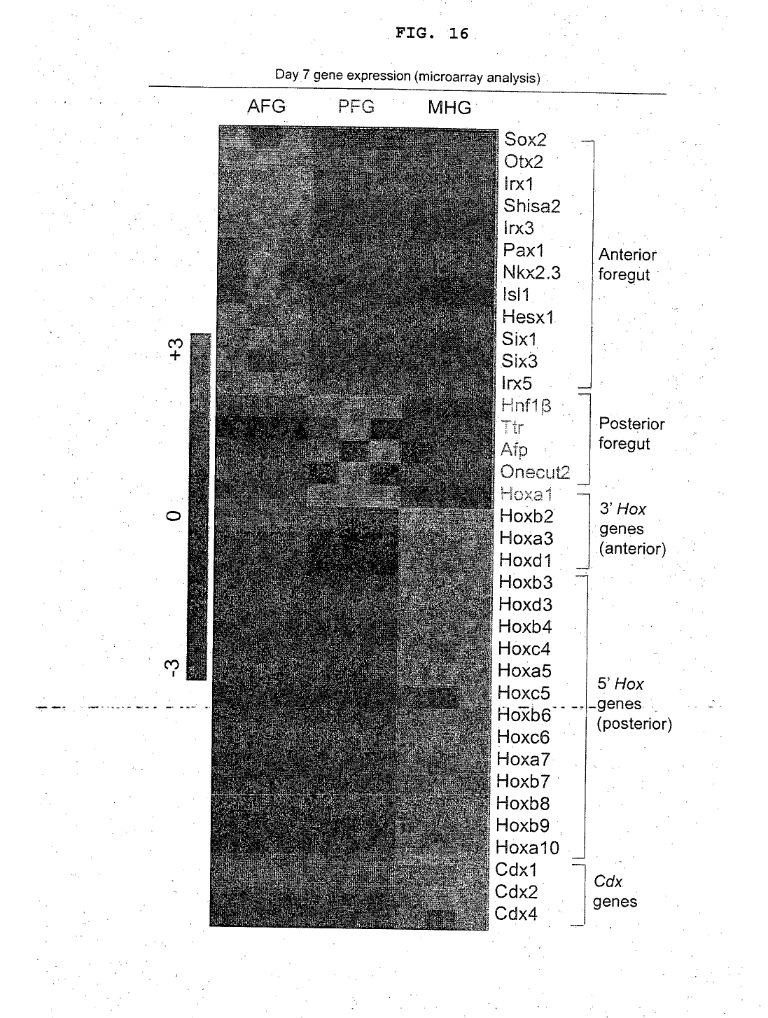

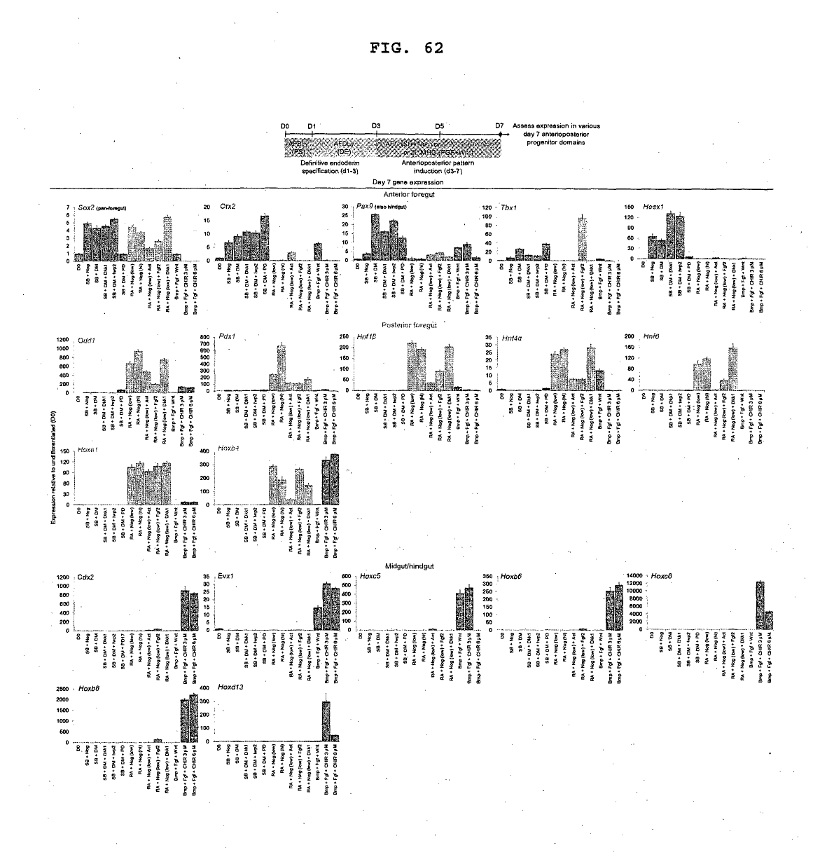

In vertebrate embryos, tailbud mesoderm expresses BMP4, FGF4/8 and WNT3A and is juxtaposed with posterior endoderm, suggesting these signals might posteriorly pattern the nearby MHG. In vitro, BMP markedly posteriorized DE (FIG. 14i), inducing MHG TFs (e.g., CDX2, EVX1 and 5′ HOX genes), mirroring zebrafish data (Tiso et al., 2002). Wnt (emulated by CHIR) was similarly posteriorizing (FIG. 14ii) and FGF could partially posteriorize PFG into MHG (FIG. 62), confirming prior work (Sherwood et al., 2011; Spence et al., 2011). Reciprocally, BMP, FGF and Wnt all suppressed anterior endoderm TF SOX2 (FIG. 14, FIG. 62). Hence a combination of BMP, CHIR and FGF was used to pattern day 3 DE into >99% CDX2⁺ MHG (FIG. 15) while suppressing foregut (FIG. 16) in serum free conditions.

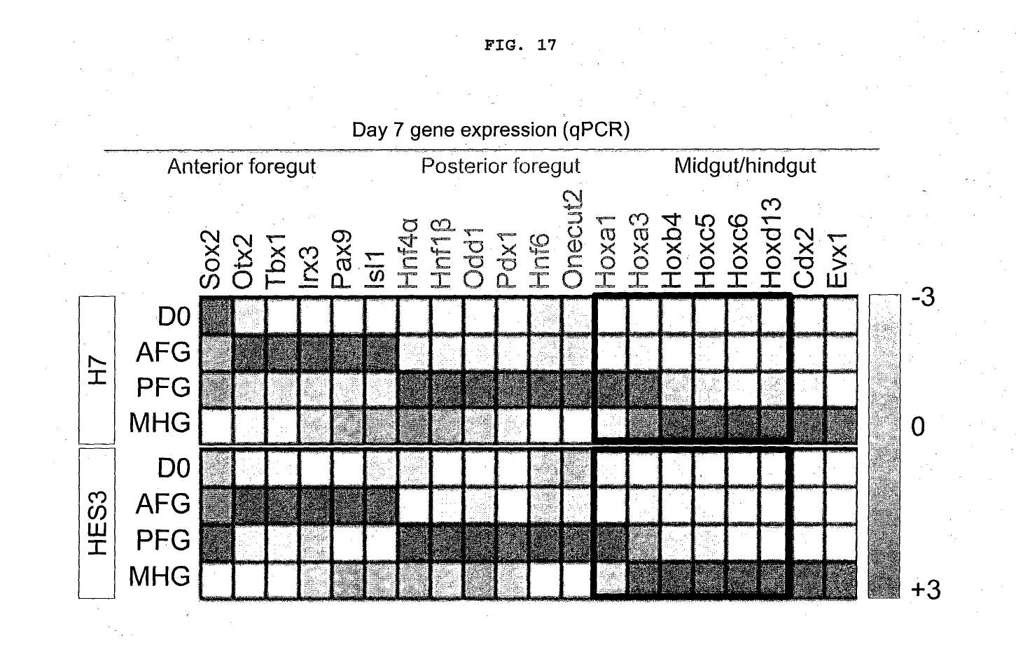

Conversely, inhibiting posteriorizing BMP signals broadly yielded anterior endoderm (foregut). Combining BMP inhibition with TGFβ inhibition (Green et al., 2011) yielded>98% OTX2⁺ AFG (FIG. 15) by day 7 of differentiation, evocative of OTX2⁺ anteriormost pharyngeal endoderm in vivo (Table 1). Separately, BMP inhibition in conjunction with RA signaling generated PFG (FIG. 16-17), consistent with how RA regionalizes the PFG in vivo (Stafford and Prince, 2002). AFG and PFG were functionally distinct, as only PFG harbored hepatic and pancreatic potential (FIG. 18), showing only PFG acquired the competence to subsequently form liver and pancreas.

Invoking the above signaling logic, separate AFG, PFG and MHG populations from DE were generated in a mutually-exclusive manner, as evinced by microarray and qPCR analyses. Anteroposterior gene expression was clearly developmentally bounded (FIG. 16-17 reproduced in 2 hESC lines). Graded, spatially collinear HOX gene expression (Zorn and Wells, 2009) was observed after in vitro patterning, whereby PFG expressed 3′ anterior HOX genes (e.g., HOXA1) and by contrast MHG exclusively expressed 5′ posterior HOX genes and CDX genes (FIG. 16-17).

TGFβ Competes with BMP/MAPK Signaling to Specify Mutually-Exclusive Bifurcation of Pancreatic and Hepatic Fates

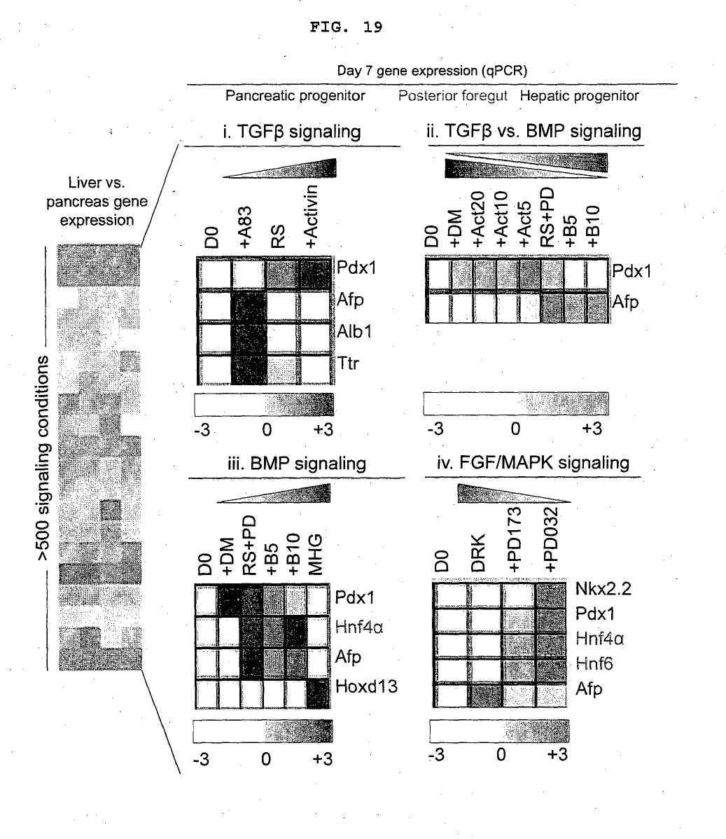

In vivo, liver and pancreas develop from a common PFG precursor that faces a binary lineage decision (Chung et al., 2008; Deutsch et al., 2001). Pancreas- and liver-inducing signals have been identified in vivo and in vitro, but how liver and pancreas might be segregated during PSC differentiation is less clear. BMP and FGF are typically used to induce liver, whereas Hedgehog inhibition and FGF are applied to generate pancreas (e.g., Cho et al., 2012; Kroon et al., 2008). A signaling perturbation analysis encompassing >500 conditions (FIG. 19, FIG. 63) clarified a signaling switch for mutually exclusive specification of pancreas versus liver (FIG. 19).

TGFβ signaling was found to promote pancreas formation (tracked by PDX1) whereas BMP and FGF/MAPK signaling specified liver (AFP) (FIG. 19). Importantly, it was clarified that each of these signals reciprocally repressed formation of the alternate lineage (FIG. 19), emphasizing how the PFG lineage decision is bistable (Chung et al., 2008). Due to such cross-repression, eliminating pro-pancreatic TGFβ reciprocally expanded liver (FIG. 19i-ii) whereas inhibition of pro-hepatic FGF/MAPK (Deutsch et al., 2001) diverted differentiation towards pancreas (FIG. 19iv). The results presented herein differ from prior work and may explain previous inefficiencies in liver or pancreas induction. Prior use of FGF for pancreatic induction (Cho et al., 2012; Kroon et al., 2008; Nostro et al., 2011) may in fact block pancreas and instead specify liver (FIG. 19iv), as suggested by embryonic studies (Deutsch et al., 2001). On the other hand, provision of TGF for hepatic induction (Rashid et al., 2010) may abrogate liver and instead drive pancreas (FIG. 19i-ii).

Mechanistically, a dichotomy in TGFβ versus BMP in respectively specifying pancreas versus liver (FIG. 20) has not been previously elucidated and is reminiscent of how these signaling pathways often cross-repress each other's transduction (Candia et al., 1997). Combinatorial interactions were further identified between these morphogens. For example, TGFβ signaling AND FGF/MAPK inhibition was essential for pancreas formation, as FGFMAPK inhibition was ineffective if TGFβ was inhibited in parallel (FIG. 63i). Conversely, hepatic induction cooperatively required TGFβ inhibition AND FGF/MAPK signaling (FIG. 19iv, FIG. 63i), as TGFβ inhibition failed to efficiently create liver if FGF/MAPK was simultaneously inhibited.

hESC-Derived Hepatic Progeny Engraft Long-Term into Unconditioned Mouse Liver

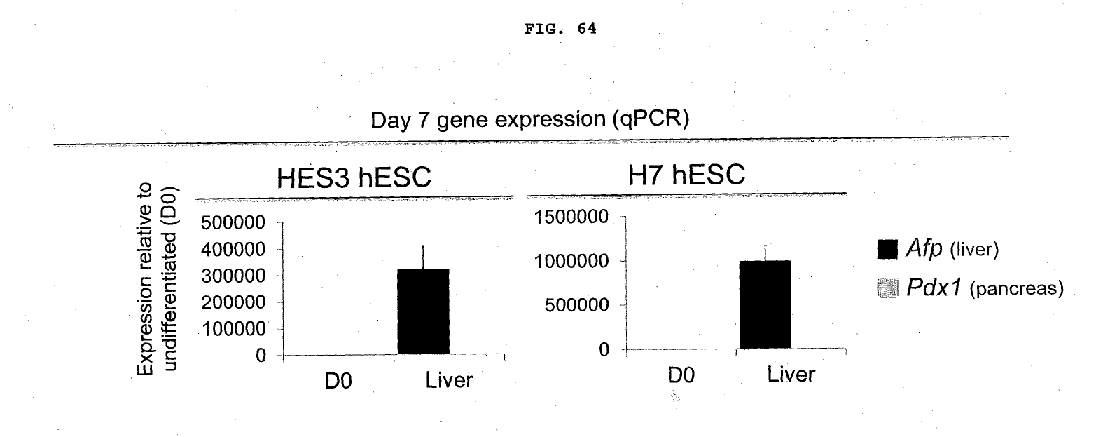

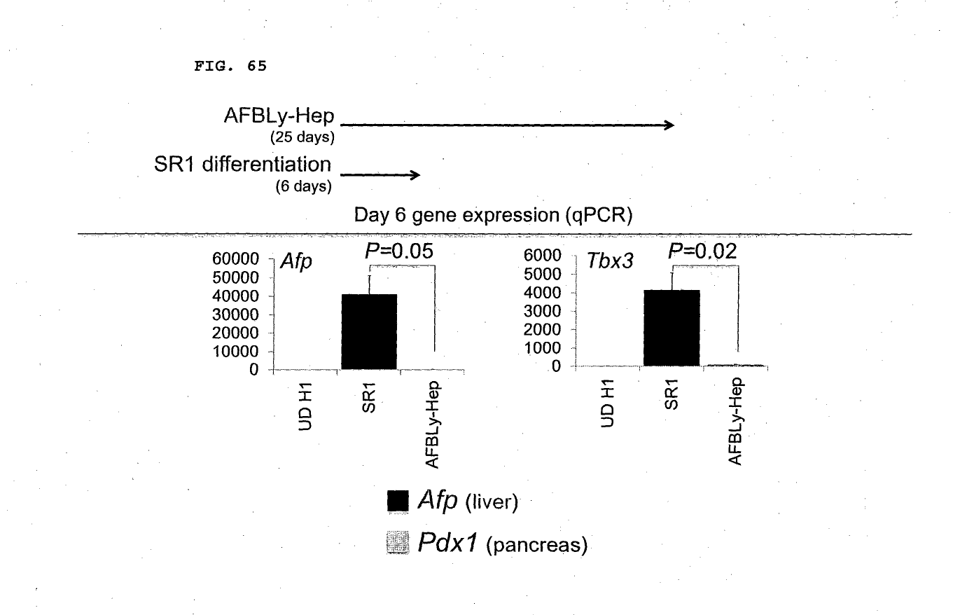

To differentiate DE towards liver while explicitly inhibiting pancreas, we induced DE towards PFG for 1 day (FIG. 20i, FIG. 63iv) and then employed TGFβ inhibition in conjunction with BMP and other factors to direct PFG towards liver over 3 subsequent days with minimal pancreatic contamination (FIG. 64). We generated 72.3±6.3% AFP⁺ early hepatic progenitors (FIG. 64) from 4 hESC lines within 7 days of differentiation, which is twice as rapid as prior methods (Rashid et al., 2010): moreover liver markers were induced ˜60-210 times higher compared to earlier protocols (FIG. 65).

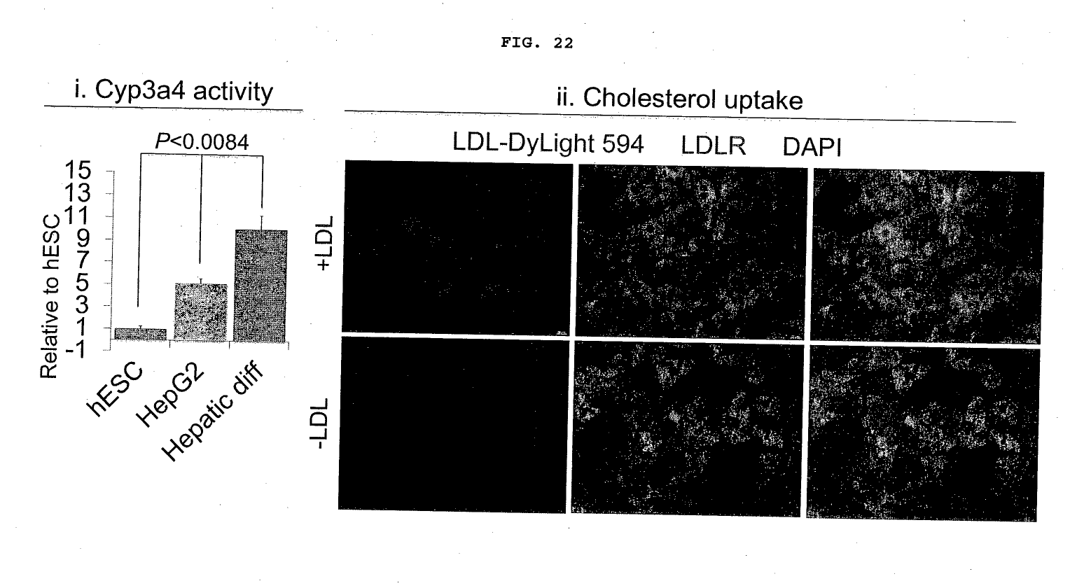

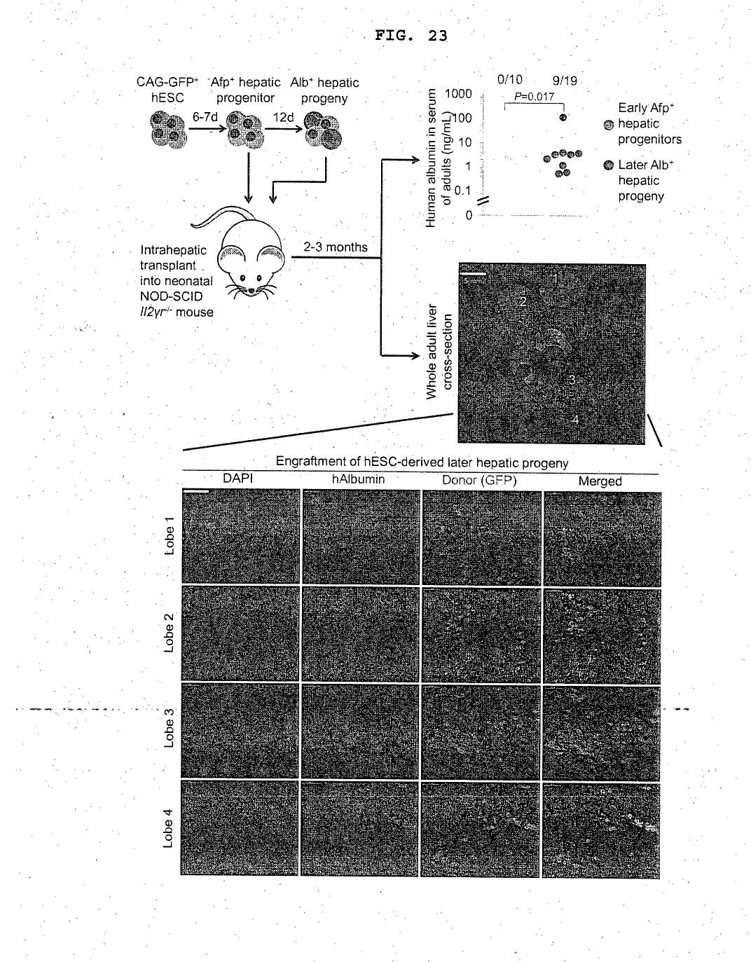

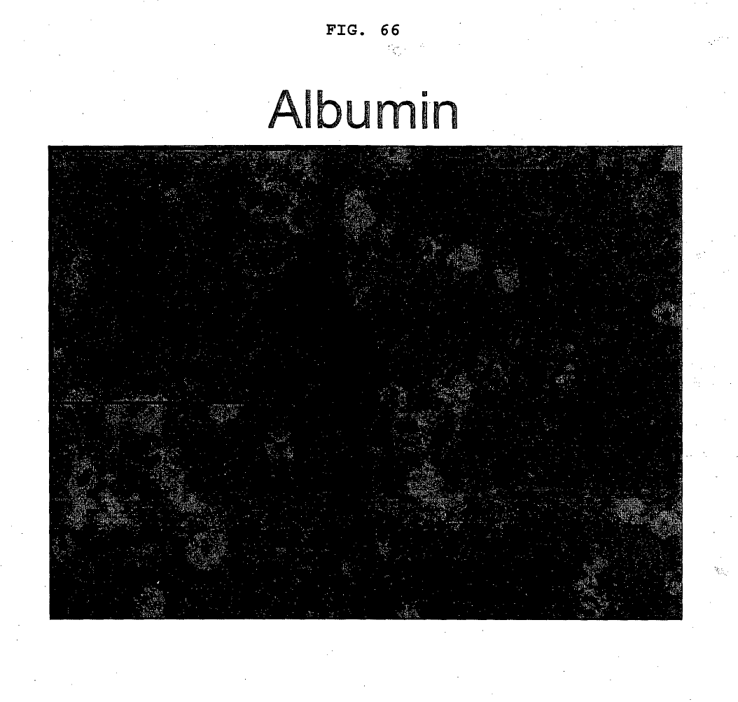

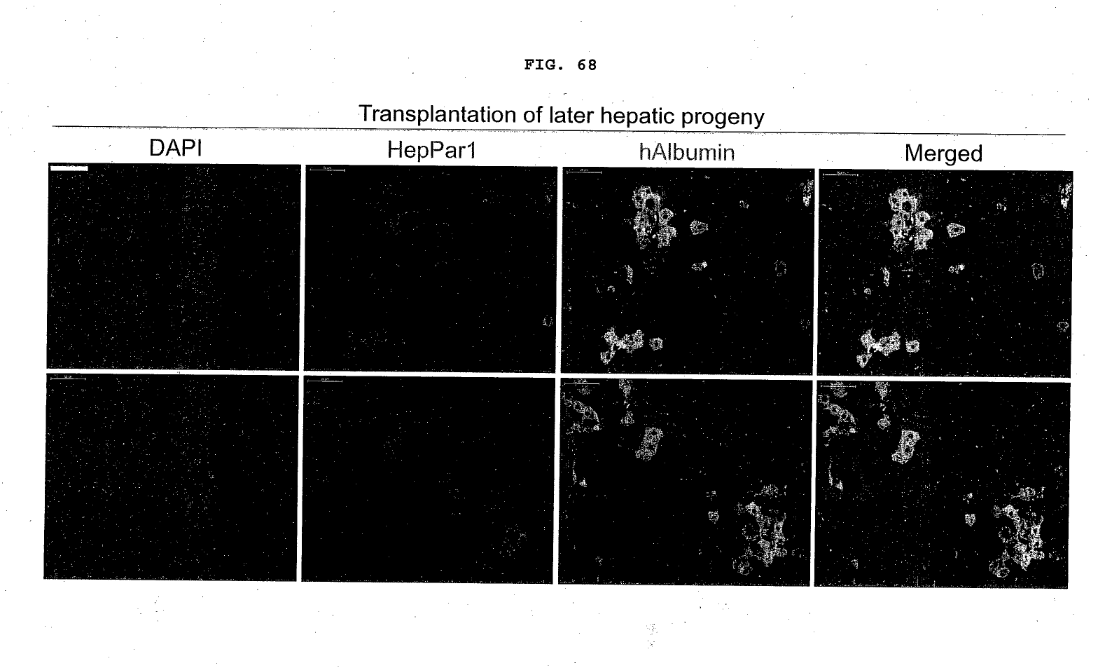

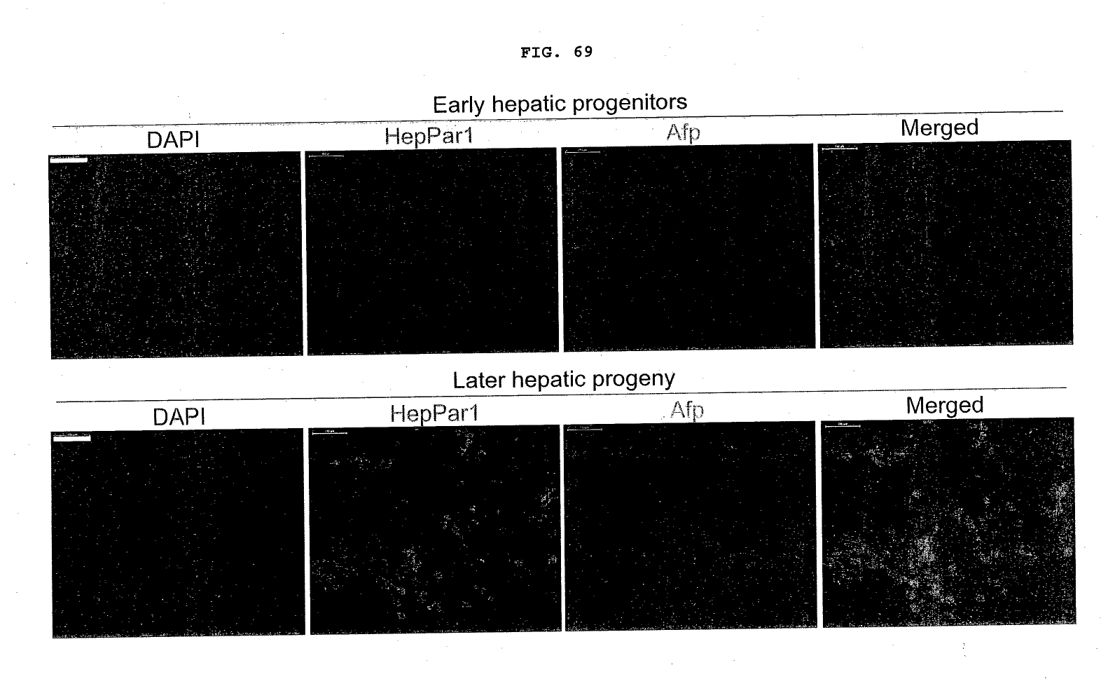

To validate the hepatic potential of early AFP⁺ liver progenitors, they were empirically matured in vitro with oncostatin M and dexamethasone (Kamiya et al., 1999) into a mixed albumin (hALB)⁺ hepatoblast population (FIG. 66), which exhibited some extent of CYP3A4 metabolic activity (FIG. 22i), expressed LDLR and could uptake cholesterol (FIG. 22ii). When transplanted into neonatal mouse livers, early AFP⁺ hepatic progenitors failed to engraft (FIG. 67), but when their differentiated hALB⁺ progeny were transplanted, human albumin was detected in the blood of 47% of recipients (mean 7.2 ng/mL as determined by the two-sided Mann-Whitney test) 2-3 months post-transplantation, indicating long-term engraftment (FIG. 23). Indeed, foci of hALB⁺ hESC-derived hepatic cells (marked with constitutively-expressed GFP prior to transplantation) were present in all lobes of the adult liver (FIG. 23, FIG. 67). This suggested hALB⁺ hepatic cells had integrated and/or migrated throughout the liver and they were not simply locally persisting at the site of transplantation. Finally, hALB⁺ cells coexpressed human hepatic marker HepPar1 (FIG. 68) but did not detectably express fetal marker AFP (FIG. 69), suggesting they had progressed past the fetal stage. This is the first demonstration that hESC-derived hepatic cells could engraft long-term into mouse livers that were not compromised by extensive pharmacologic or genetic damage (cf. Yusa et al., 2011).

### Comprehensive Transcriptional and Chromatin State Mapping of Endoderm Induction and Anteroposterior Patterning

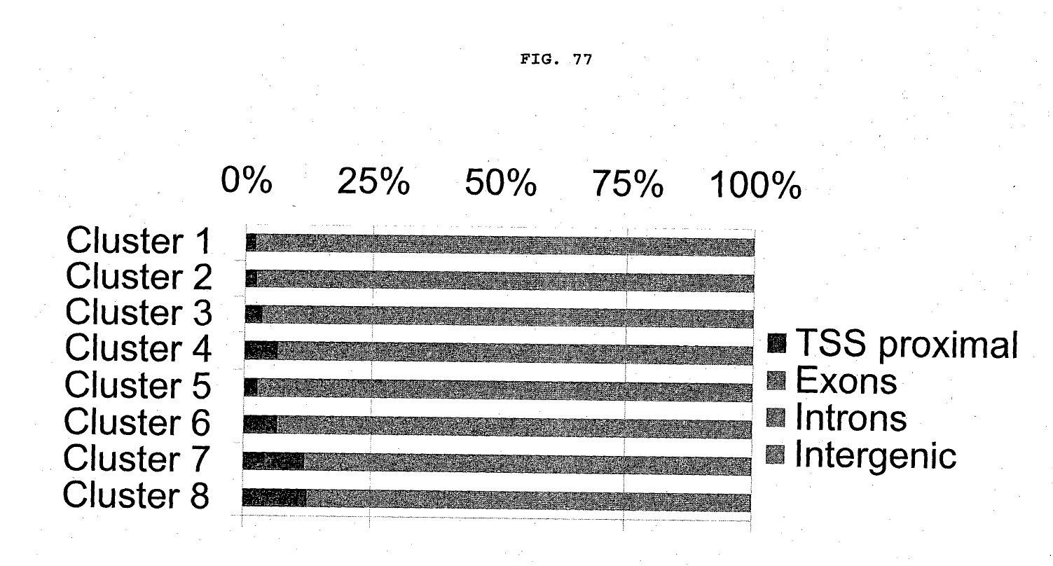

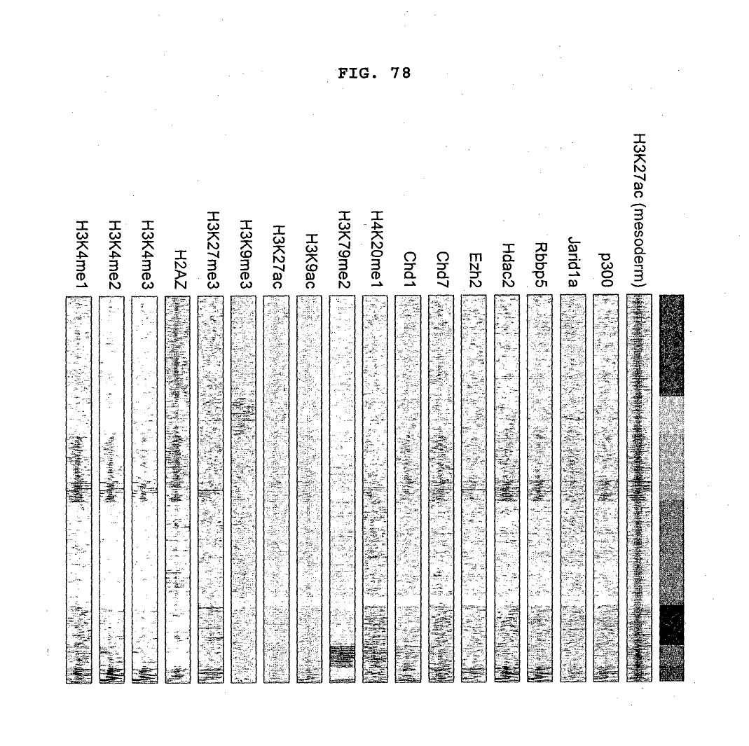

Capitalizing on the ability to obtain rather homogenous populations of hESC-derived endodermal lineages, transcriptional and chromatin dynamics were captured during endoderm development by profiling a hierarchy of six pure progenitor populations (hESC, APS, DE, AFG, PFG and MHG) using RNA-seq and ChIP-seq for 4 histone H3 modifications (K4me3, K27me3, K27ac and K4me2; FIG. 24-38, FIG. 66-80). This yielded transcriptional and chromatin state maps spanning 4 embryonic stages (epiblast, PS, DE and anteroposterior patterning) totaling >1.3 billion aligned reads (FIG. 70).

The analyses captured acute developmental transitions. RNA-seq revealed dramatic transcriptional changes within 24 hours during synchronous transit from pluripotency to APS in vitro (FIG. 24), mirroring how epiblast (˜E5.5) and PS (˜E6.5) arise within 1 day in the mouse. The BRACHYURY and NODAL promoters were bivalently marked by activation-associated K4me3 and repression-associated K27me3 in hESC, yet within 24 hours of APS induction they were unilaterally resolved, losing repressive K27me3 and gaining active marks K27ac and K4me3 concomitant with rapid BRACHYURY and NODAL upregulation in APS (FIG. 25).

Endoderm Enhancer Activation is Associated with EOMES, SMAD2/3/4 and FOXH1 Co-Occupancy

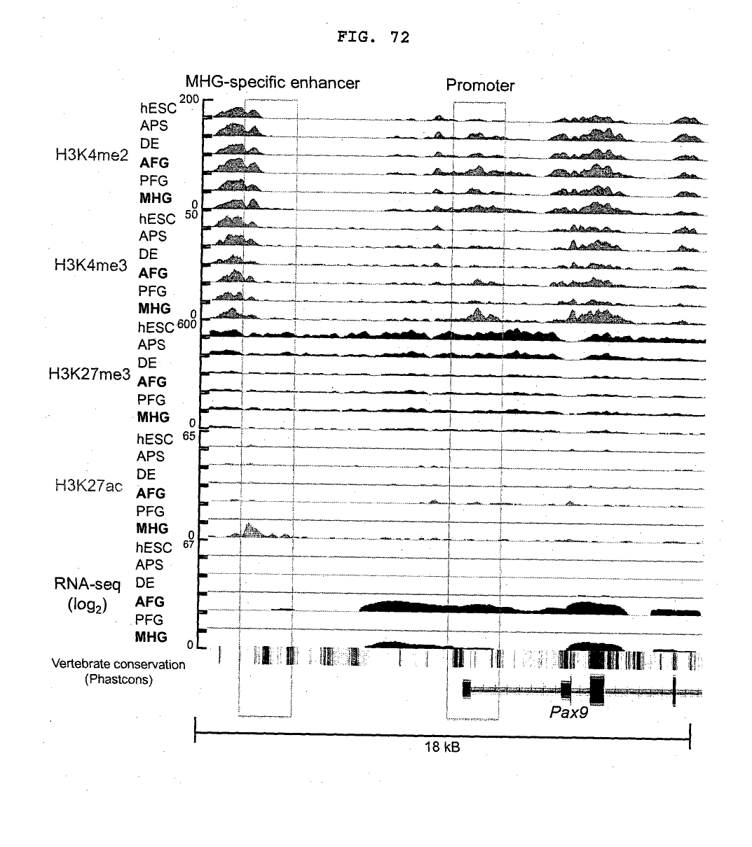

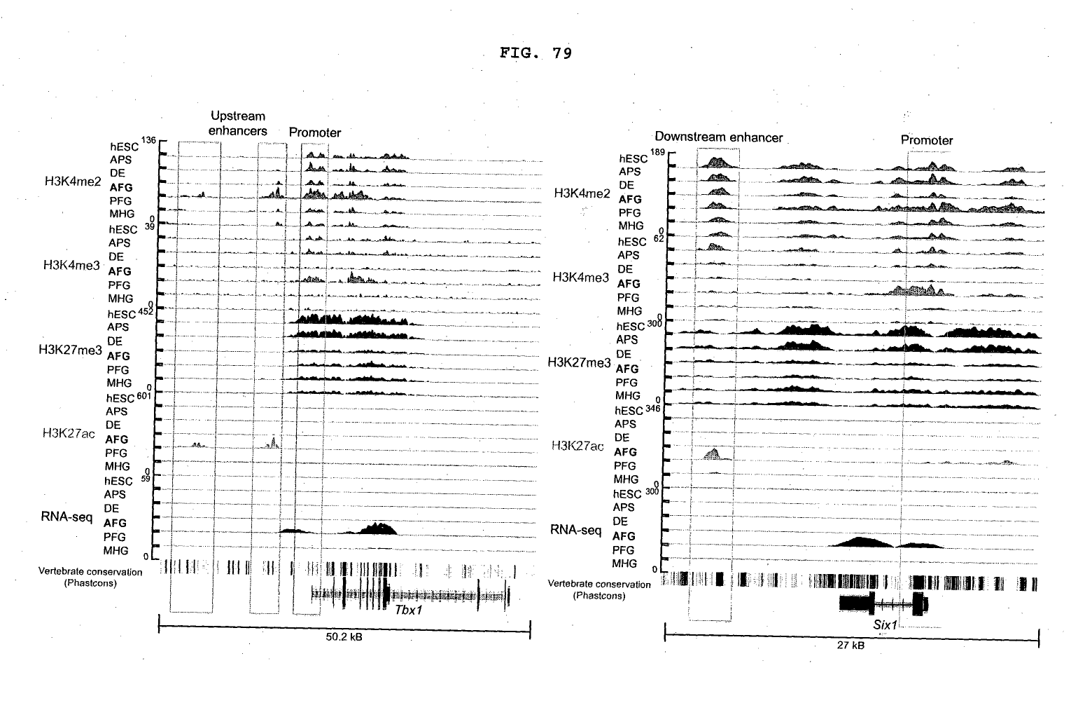

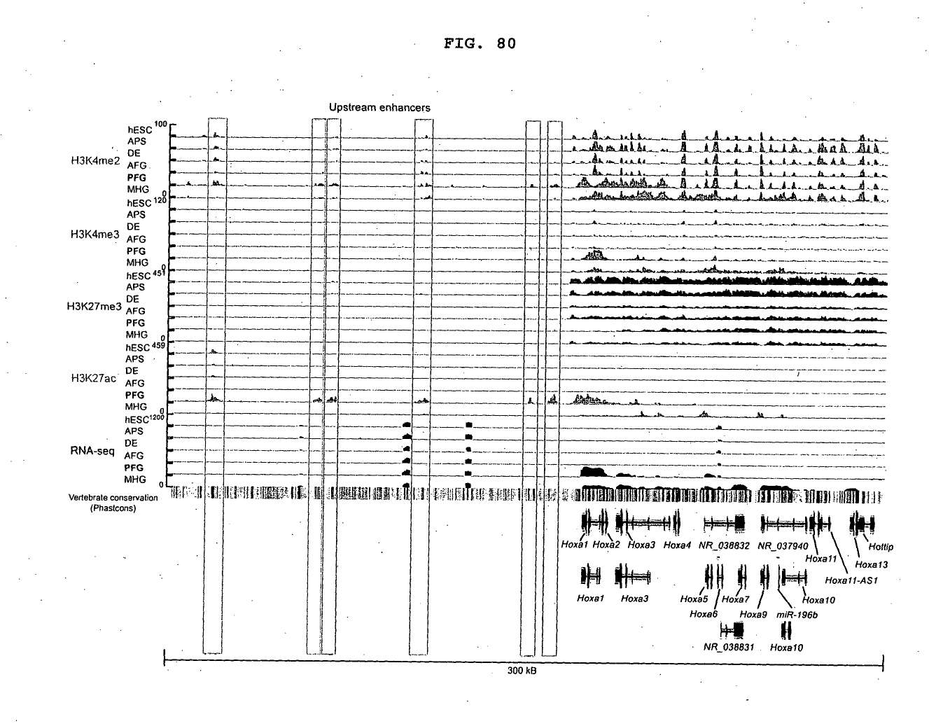

Distinct batteries of active enhancers identified by distal K27ac enrichment (Rada-Iglesias et al., 2011) were invoked during each cell-fate transition (FIG. 26). APS enhancers (e.g., BRACHYURY and NODAL) were rapidly inaugurated within 24 hours (FIG. 25). During DE patterning, distinct cohorts of enhancers were commissioned in each anteroposterior domain in AFG (SIX1 and TBX1; FIG. 79), PFG (HOXA1; FIG. 80) and MHG (CDX2 and PAX9; FIG. 27, FIG. 72).

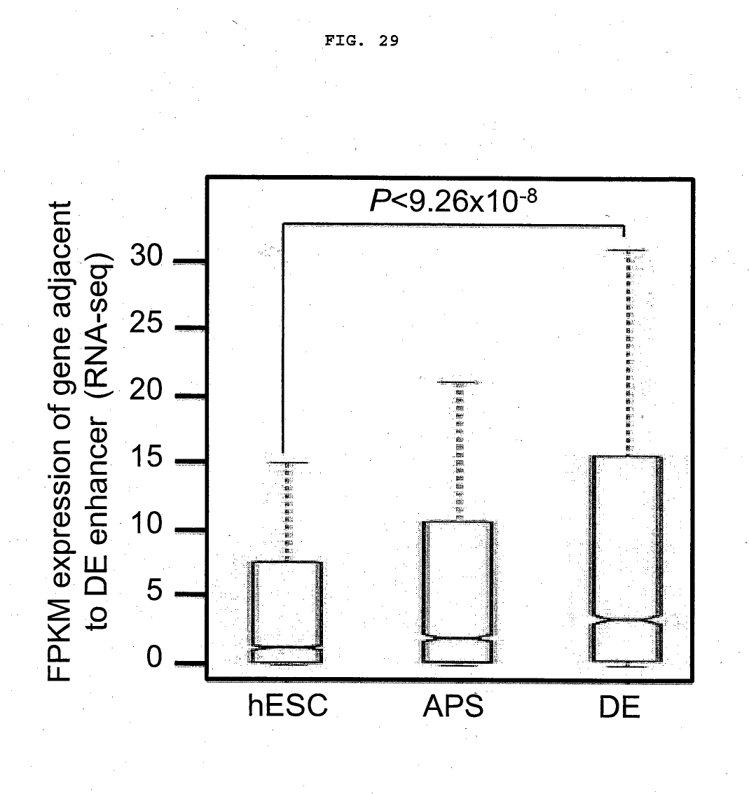

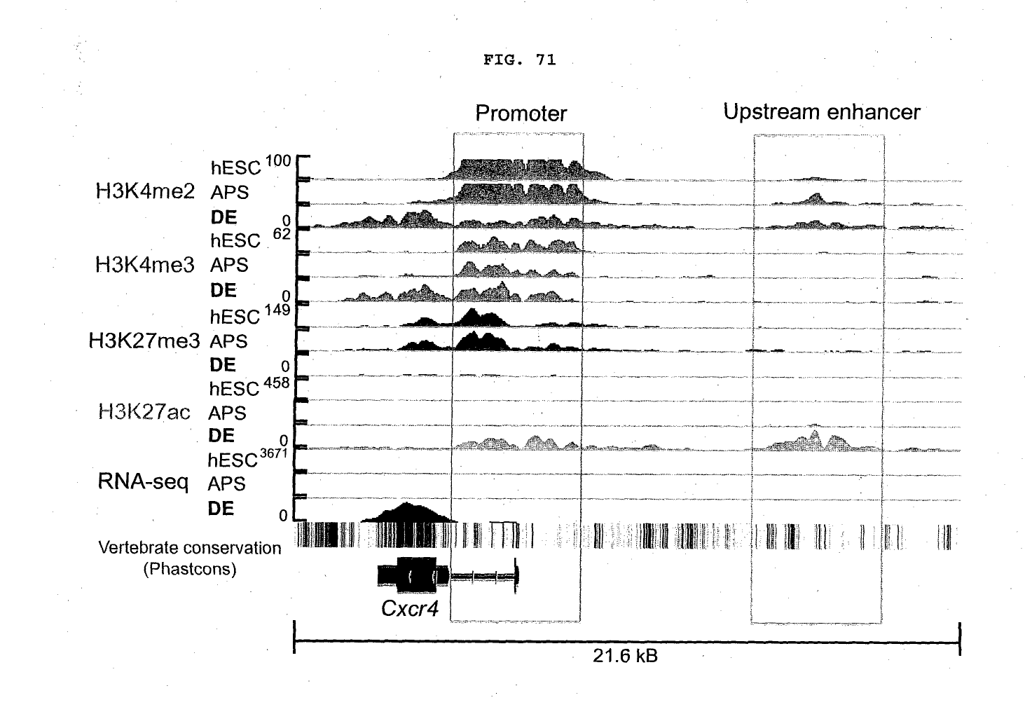

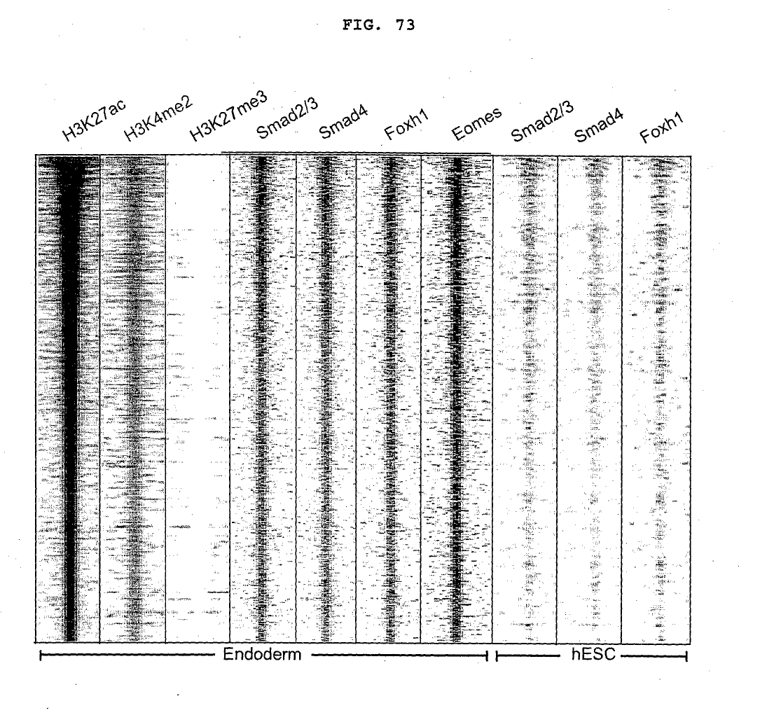

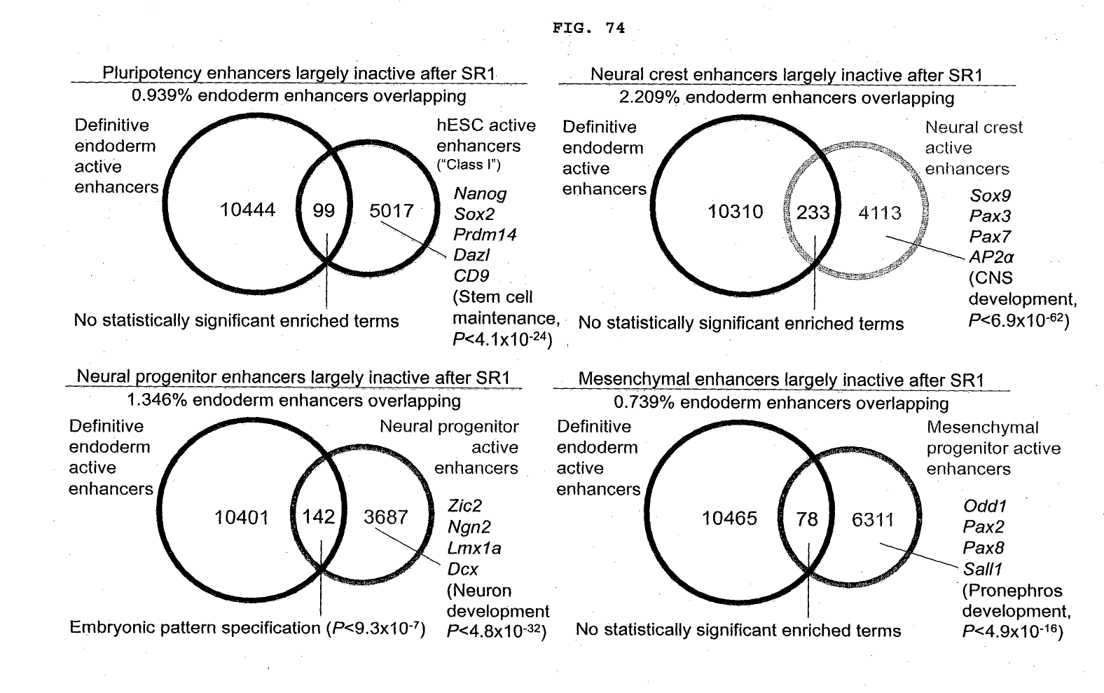

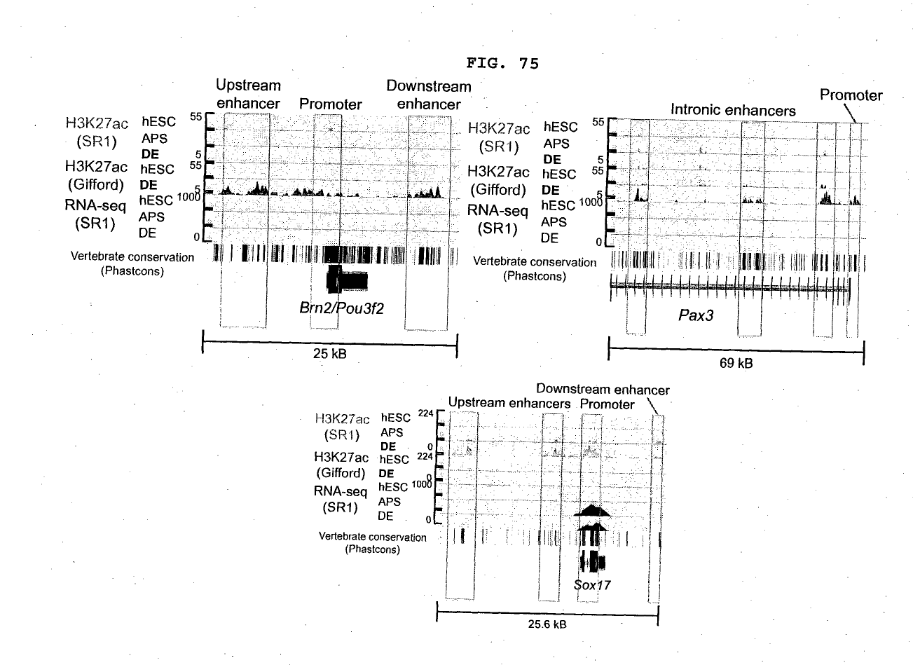

10,543 DE enhancers were activated upon DE specification, gaining K27ac despite being largely inactive in hESC. Active DE enhancers flanked archetypic DE regulators, e.g. SOX17 (FIG. 34) and CXCR4 (FIG. 71). Gene ontology (GO) analyses (McLean et al., 2010) associated these enhancers most significantly with endoderm development (P<3.84×10⁻²⁶) and gastrulation (P<7.92×10⁻²⁶; FIG. 28), affirming the purity of differentiated DE populations. Genes adjacent to active DE enhancers were upregulated in gastrula-stage endoderm in vivo (P<1.38×10⁻³⁹, FIG. 28) and upon DE differentiation in vitro (FIG. 29). Active DE enhancers coincided with euchromatic mark K4me2 (FIG. 73), were devoid of repression-associated K27me3 (FIG. 73), were evolutionarily conserved (FIG. 75) and were broadly inactive in other lineages (FIG. 74).

DE enhancers previously remained elusive because most prior work only assessed promoter marks (Kim et al., 2011; Xie et al., 2013). However, enhancer profiling of hESC-derived DE was recently reported (Gifford et al., 2013) and therefore our two DE datasets were compared using identical analytic methods. Paradoxically, DE enhancers from the former dataset (Gifford et al., 2013) were highly enriched for neural functions (P<3.93×10⁻²⁸; FIG. 31), as enhancers for neural TFs BRN2 and PAX3 were activated, but SOX17 enhancers were virtually silenced (FIG. 75). Association of DE enhancers with neural genes led to the prior conclusion that endoderm and ectoderm development are related (Gifford et al., 2013), which contrasts with the in vivo order of germ layer segregations (cf. Tzouanacou et al., 2009). By contrast, neural terms were largely absent in SR1-derived DE (FIG. 28) and ultimately only 4.8% of DE enhancers were shared between our and their datasets. Thus, molecular profiling of mixed DE populations (potentially enriched for ectoderm; Gifford et al., 2013) has precluded accurate molecular description of endoderm development.

How DE enhancers are inaugurated during differentiation remains obscure. Motifs for multiple TFs, including DE specifiers EOMES and FOXA2 as well as TGFβ signaling effectors SMAD2/3 and FOXH1 (P=10⁻⁵⁹-10⁻¹⁹⁷) were enriched in DE enhancers (FIG. 32), consistent with how these TFs specify DE in vivo (e.g., Dunn et al., 2004; Teo et al., 2011). Interestingly, we found EOMES, SMAD2/3, SMAD4 and FOXH1 (Kim et al., 2011; Teo et al., 2011) co-occupied an extensive series of DE enhancers (FIG. 33), including the SOX17 enhancer (FIG. 34). Although EOMES individually engaged some elements, colocalization of EOMES with TGFβ signaling effectors SMAD2/3/4 and FOXH1 correlated with maximal enhancer acetylation (FIG. 33, P<10⁻³⁰⁰ as calculated by Fisher's exact t test, 4 TFs vs. 1-3 TF classes). Thus, convergence of both lineage-specifying and signaling-effector TFs may propel full-fledged enhancer activation upon differentiation (Calo and Wysocka, 2013).

### Endoderm Enhancers Reside in a Diversity of “Pre-Enhancer” States in Uncommitted Cells Prior to Activation

It remains unclear how DE enhancers are so swiftly engaged upon hESC differentiation. SMAD2/3/4 and FOXH1 occupy DE enhancers upon differentiation but infrequently do so in the uncommitted state (FIG. 73). Perhaps these enhancers are instead primed for activation at the level of chromatin. Premarking of developmental enhancers by euchromatic K4me1 in ESC signifies a “window of opportunity” for subsequent enhancer activation (Calo and Wysocka, 2013; Rada-Iglesias et al., 2011). Developmental progression was reviewed, assessing occupancy of DE enhancers by >24 histone modifications and chromatin regulators (Ernst et al., 2011) in hESC prior to enhancer activation (FIG. 35). Unexpectedly, K4me1 labeled less than one third of future DE enhancers in hESC, implying “poising” by K4me1 in hESC is not always essential for immediate enhancer activation (FIG. 35-36). Thus, we sought to systematically discover all possible “pre-enhancer” chromatin states of DE enhancers in hESC.

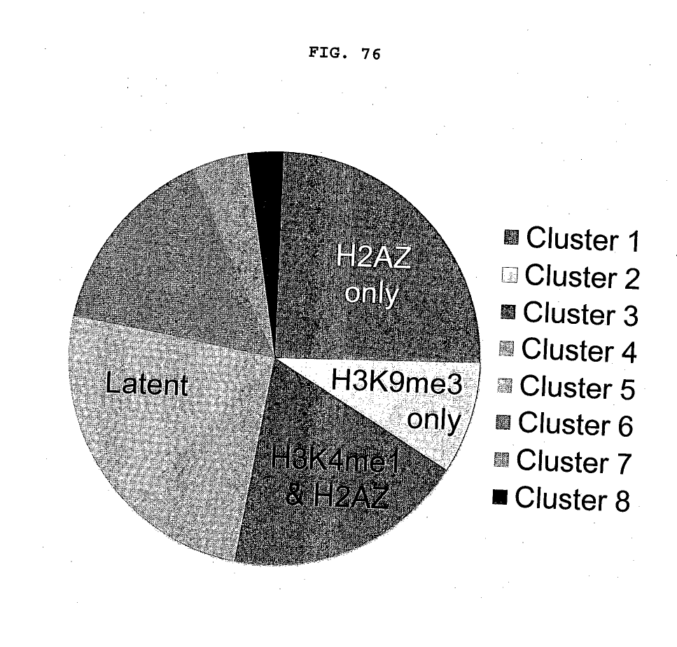

Unsupervised clustering revealed 25% of DE enhancers existed in a novel pre-enhancer state (cluster 1) in hESC largely defined by histone variant H2AZ and no other known chromatin marks (FIG. 35, FIG. 76). Despite virtual absence of K4me1, H2AZ-marked pre-enhancers became rapidly activated within 3 days of DE induction (FIG. 35). DE enhancers less frequently resided in a repressed state designated by heterochromatic mark K9me3 (cluster 2) (Zhu et al., 2012) or a “latent” pre-enhancer state largely lacking known histone modifications (cluster 5, FIG. 35) (Ostuni et al., 2013). Only 10% of DE enhancers were marked by K27me3 in hESC (FIG. 36); suggesting Polycomb (Rada-Iglesias et al., 2011) was not always necessary to repress developmental enhancers in hESC: perhaps absence of K27ac/histone acetyltransferases (HATs) was sufficient to confer inactivity. Only a minority of DE enhancers (10%) were pre-loaded with HAT p300 (Rada-Iglesias et al., 2011) (FIG. 36), suggesting rapid enhancer acetylation during differentiation may largely involve de novo HAT recruitment.

A “pre-enhancer” state solely delineated by H2AZ without other detectable distinguishing factors has not been previously described. H2AZ is often associated with active enhancers (Hu et al., 2013), yet it was found that it also decorated inactive enhancers (FIG. 35). H2AZ-laden nucleosomes are unstable and are readily displaced by TFs (Hu et al., 2013; Jin et al., 2009). This may permit endoderm TFs to rapidly infiltrate DE enhancers upon differentiation, explaining rapid enhancer activation. Indeed, H2AZ-marked DE pre enhancers in hESC more readily attracted EOMES, SMAD2/3/4 and FOXH1 upon differentiation (FIG. 37, P=10⁻¹³-10⁻¹⁵) compared to latent pre-enhancers. This parallels how H2AZ-marked promoters in mESC are more susceptible to FOXA2 binding upon differentiation (Li et al., 2012).

In sum, initial K4me1 “poising” does not represent the only predictor of subsequent enhancer activation. The data show there is a diversity of pre-enhancer states characterized by different combinations of chromatin marks (FIG. 38).

### DISCUSSION

PSC differentiation typically yields a range of developmental outcomes that vary between PSC lines. Here is shown precise induction of a single lineage can be achieved by understanding how alternate fates are excluded at developmental branch points and by dissecting the precise temporal kinetics of dynamic signaling transitions. We delineated the signaling logic for induction and anteroposterior patterning of human endoderm from PSC and for subsequent bifurcation of pancreas versus liver, clarifying separation of alternate lineages at each step. Such knowledge enabled universal generation of purified endoderm from diverse hESC/hiPSC lines. This level of endodermal purity enabled accurate chromatin state analysis of endoderm development and production of long-term-engrafting hESC-derived liver cells.

### Developmental Segregation of Mutually Exclusive Endodermal Fates

BMP, FGF, TGFβ and Wnt signals have, been used to elicit both endoderm and mesoderm from PSC (Bernardo et al., 2011; Cheng et al., 2012; Gertow et al., 2013; Nostro et al., 2011; Touboul et al., 2010) and therefore the exact lineage outcomes driven by these signals has remained ambiguous. These contradictory findings are reconciled here, showing these factors indeed specify either endoderm or mesoderm based on their temporal kinetics. Throughout 4 successive stages of endoderm development, the signals that instruct or repress a given lineage have been accurately defined, providing a clearer view of how endodermal lineage bifurcations are driven. In fact, this refined understanding suggested that previous protocols provided incorrect signals that repressed DE formation, thereby resulting in inefficient differentiation.

This disclosure attempted to resolve a unified signaling “roadmap” spanning several consecutive stages of endoderm development, which enabled us to rationally exclude alternate fates at every stage following the hierarchy of germ layer segregations in vivo (Tzouanacou et al., 2009). Thus unraveling the signaling logic underlying DE specification has enabled systematic generation of highly pure DE populations from diverse hESC and hiPSC lines in serum-free conditions, without extraneous lineages that typically arise from current differentiation strategies. For example, DE generated in the virtual absence of mesoderm or ectoderm. It was found that combined BMP, FGF, TGFβ and Wnt signaling (Bernardo et al., 2011; Blauwkamp et al., 2012; Gadue et al., 2006) was necessary to specify APS (>99% MIXL1⁺) and repress ectoderm (Murry and Keller, 2008), rescinding ectoderm competence within 24 hours of APS induction. After ectoderm exclusion, mesoderm was sequentially eliminated by BMP inhibition, which when combined with TGFβ and FGF signaling (Bernardo et al., 2011; D'Amour et al., 2005) exclusively drove PS towards DE. Critically, it was essential to suppress endogenous BMP and Wnt signaling within PS to achieve pure DE populations. Nuances were also clarified in the interpretation of combinations of signals, showing that reception of one signal altered the response to others. For example, while BMP inhibition typically eradicated mesoderm, if DE-inducing FGF was blocked in parallel, mesoderm formation was re-enabled. Thus, FGF was obligatory to consolidate DE commitment.

Following PFG formation, TGFβ and BMP signaling dueled to specify pancreas versus liver. The inductive signal specifying one fate bilaterally cross-repressed the alternate fate, reminiscent of bistable lineage assignment during embryogenesis (Graf and Enver, 2009) has been illustrated. Therefore, efficient liver induction required TGFβ inhibition to eliminate pancreatic fates in conjunction with BMP and FGF/MAPK to positively drive liver and vice versa. In sum, inhibition of repressive signals is equally important as provision of inductive signals to induce efficient hPSC differentiation at each branchpoint.

Elimination of alternate fates at each juncture defined a single effective strategy to universally differentiate 7 diverse hESC/hiPSC lines into highly-pure DE populations, without extraneous lineages typically induced by earlier protocols. This is contrary to the notion that different hPSC lines have distinct differentiation biases and each might require customized signals to drive efficient commitment. The observations herein are timely as a prerequisite for cell replacement therapy is the generation of homogeneous human lineages from hPSC under defined, serum-free conditions (Cohen and Melton, 2011; McKnight et al., 2010). Recent strategies to generate “self-renewing” DE (Cheng et al., 2012) or liver buds (Takebe et al., 2013) from hPSC are appealing; however they require coculture with heterologous feeders and thus suit a different type of application.

### Obligatory Endodermal Signaling Inputs are Highly Temporally Dynamic

Despite the importance of dynamic endodermal signaling transitions in vivo and in vitro (Green et al., 2011; Wandzioch and Zaret, 2009), the precise sequence and kinetics of such signals remain to be fully elucidated. For example, BMP and Wnt have been traditionally associated with mesoderm induction through studies of prolonged treatment over several days (Bernardo et al., 2011; Gadue et al., 2006). However it was found that BMP and Wnt initially specified APS, but within 24 hours of differentiation, signaling requirements were reversed such that BMP and Wnt repressed DE generation from PS and instead induced mesoderm. Critically, prior schema reduced APS and DE induction into a single lengthy stage and persistently provided BMP for 3-5 days (Nostro et al., 2011; Touboul et al., 2010), likely generating contaminating mesoderm at later stages and inhibiting DE formation. Notably, the striking temporal dynamism with which BMP and Wnt were interpreted in vitro (within 24 hours) precisely tracked how epiblast, PS and DE arise within 24 hours of one another in the mouse embryo. Therefore, assigning BMP and Wnt as either pro- or anti-endoderm is a misnomer because these signals are dynamically interpreted to yield dichotomous outcomes within just 24 hours.

### Developmental Competence and a Diversity of Pre-Enhancer States

Since Waddington's formalism of developmental competence (Waddington, 1940), its molecular basis has remained cryptic. Permissive chromatin priming of developmental enhancers in uncommitted cells may foreshadow competence (Calo and Wysocka, 2013). Various models proposed enhancers primed for rapid induction resided in either “poised” or “latent” chromatin states prior to activation (Ostuni et al., 2013; Rada-Iglesias et al., 2011). However, the relative prevalence of “poised” or “latent” pre-enhancer states (and whether they represented all possible pre-enhancer states) remained uncertain.

The chromatin state of all prospective DE enhancers in hESC was interrogated using unsupervised clustering. It was found that individual DE enhancers existed in a wide continuum of differentially-marked pre-enhancer states prior to activation, extending beyond “poised” or “latent” states. Only a subset of DE enhancers were pre-marked by K4me1, K27me3, p300 or other proposed “poising” factors in hESC, showing there is no universal poising signature.

Strikingly, it was found that many prospective DE enhancers were marked exclusively by H2AZ in the general absence of other “open” or “closed” chromatin marks. This complements recent findings that H2AZ is functionally essential for DE induction from mESC and that its presence at promoters correlated with increased FOXA2 recruitment (Li et al., 2012). It was revealed that H2AZ is sometimes the earliest recognizable enhancer mark (in lieu of K4me1). Therefore, there are multiple possible sequences of chromatin alterations during enhancer activation and hence there is no universal initial enhancer “poising” event. It was further inferred that H2AZ prepositioning at DE enhancers enabled optimal infiltration by lineage specifier EOMES and signaling effectors SMAD2/3/4 and FOXH1 upon differentiation and that co-occupancy by all these TFs correlated with maximal enhancer activation. Altogether, this demonstrated that the primordial chromatin state of a DE enhancer in hESC could influence its future engagement upon differentiation.

Delineating the developmental signaling logic underlying multiple successive endodermal lineage bifurcations has proven decisive in universally generating purified endoderm from diverse hPSC lines. Generation of any committed cell-type occurs through the intermediacy of multiple precursors: therefore, highly-efficient differentiation at each intermediate stage is necessary to obtain enriched yields of the final product (McKnight et al., 2010). We show highly-pure endoderm populations are the obligatory foundation to generate engraftable endodermal derivatives and to obtain accurate molecular insights into endoderm commitment. Heterogeneity in DE populations has previously yielded miscellaneous cell-types and obscured molecular signatures of DE differentiation. Developmental TF and cell-surface marker expression, chromatin state analyses and tests of restricted developmental competence all illustrate and confirm the purity and identity of endodermal populations produced in this work.

In summary, the disclosure herein provides a coherent view of signaling logic and chromatin dynamics propelling endoderm specification and patterning, therefore availing hPSC differentiation and enriching the knowledge of human developmental biology from a unique perspective. Specifically, the observations made from hPSC differentiation will reciprocally enrich our knowledge of developmental biology from a unique perspective. The signaling Perturbations, transcriptional profiling and chromatin analyses reported here respectively illuminate the extrinsic signals, regulatory, genes and genomic regulatory elements that animate human endoderm development.

### REFERENCES

Ang, S. L., Conlon, R. A., Jin, O., and Rossant, J. (1994). Positive and negative signals from mesoderm regulate the expression of mouse Otx2 in ectoderm explants. Development 120, 2979-2989.
Ang, S. L., Wierda, A., Wong, D., Stevens, K. A., Cascio, S., Rossant, J., and Zaret, K. S. (1993). The formation and maintenance of the definitive endoderm lineage in the mouse: involvement of HNF3/forkhead proteins. Development 119, 1301-1315.
Avilion, A. A., Nicolis, S. K., Pevny, L. H., Perez, L., Vivian, N., and Lovell-Badge, R. (2003). Multipotent cell lineages in early mouse development depend on SOX2 function. Genes & Development 17, 126-140.
Bain, J., Plater, L., Elliott, M., Shpiro, N., Hastie, C. J., McLauchlan, H., Klevernic, I., Arthur, J. S. C., Alessi, D. R., and Cohen, P. (2007). The selectivity of protein kinase inhibitors: a further update. Biochem J 408, 297-315.
Barnes, J. D., Crosby, J. L., Jones, C. M., Wright, C. V., and Hogan, B. L. (1994). Embryonic expression of Lim-1, the mouse homolog of Xenopus Xlim-1, suggests a role in lateral mesoderm differentiation and neurogenesis. Developmental Biology 161, 168-178.
Beck, F., Erler, T., Russell, A., and James, R. (1995). Expression of Cdx-2 in the mouse embryo and placenta: possible role in patterning of the extra-embryonic membranes. Dev Dyn 204, 219-227.
Becker, D., Jiang, Z., Knödler, P., Deinard, A. S., Eid, R., Kidd, K. K., Shashikant, C. S., Ruddle, F. H., and Schughart, K. (1996). Conserved regulatory element involved in the early onset of Hoxb6 gene expression. Dev Dyn 205, 73-81.
Bell, E., Ensini, M., Gulisano, M., and Lumsden, A. (2001). Dynamic domains of gene expression in the early avian forebrain. Dev Biol 236, 76-88.
Beppu, H., Kawabata, M., Hamamoto, T., Chytil, A., Minowa, O., Noda, T., and Miyazono, K. (2000). BMP type II receptor is required for gastrulation and early development of mouse embryos. Dev Biol 221, 249-258.
Bernardo, A. S., Faial, T., Gardner, L., Niakan, K. K., Ortmann, D., Senner, C. E., Callery, E. M., Trotter, M. W., Hemberger, M., Smith, J. C., et al. (2011). BRACHYURY and CDX2 mediate BMP-induced differentiation of human and mouse pluripotent stem cells into embryonic and extraembryonic lineages. Cell Stem Cell 9, 144-155.
Biben, C., Stanley, E., Fabri, L., Kotecha, S., Rhinn, M., Drinkwater, C., Lah, M., Wang, C. C., Nash, A., Hilton, D., et al. (1998). Murine cerberus homologue mCer-1: a candidate anterior patterning molecule. Developmental Biology 194, 135-151.
Blauwkamp, T. A., Nigam, S., Ardehali, R., Weissman, I. L., and Nusse, R. (2012). Endogenous Wnt signalling in human embryonic stem cells generates an equilibrium of distinct lineage-specified progenitors. Nat Commun 3, 1070.
Blum, M., Gaunt, S. J., Cho, K. W., Steinbeisser, H., Blumberg, B., Bittner, D., and De Robertis, E. M. (1992). Gastrulation in the mouse: the role of the homeobox gene goosecoid. Cell 69, 1097-1106.
Bosse, A., Zülch, A., Becker, M. B., Torres, M., Gómez-Skarmeta, J. L., Modolell, J., and Gruss, P. (1997). Identification of the vertebrate Iroquois homeobox gene family with overlapping expression during early development of the nervous system. Mech Dev 69, 169-181.
Calo, E., and Wysocka, J. (2013). Modification of enhancer chromatin: what, how, and why? Molecular Cell 49, 825-837.
Candia, A. F., Watabe, T., Hawley, S. H., Onichtchouk, D., Zhang, Y., Derynck, R., Niehrs, C., and Cho, K. W. (1997). Cellular interpretation of multiple TGF-β signals: intracellular antagonism between activin/BVg1 and BMP-2/4 signaling mediated by Smads. Development 124, 4467-4480.
Chambers, S. M., Fasano, C. A., Papapetrou, E. P., Tomishima, M., Sadelain, M., and Studer, L. (2009). Highly efficient neural conversion of human ES and iPS cells by dual inhibition of SMAD signaling. Nat Biotechnol 27, 275-280.
Chapman, D. L., Agulnik, I., Hancock, S., Silver, L. M., and Papaioannou, V. E. (1996a). Tbx6, a mouse T-Box gene implicated in paraxial mesoderm formation at gastrulation. Developmental Biology 180, 534-542.
Chapman, D. L., Garvey, N., Hancock, S., Alexiou, M., Agulnik, S. I., Gibson-Brown, J. J., Cebra-Thomas, J., Bollag, R. J., Silver, L. M., and Papaioannou, V. E. (1996b). Expression of the T-box family genes, Tbx1-Tbx5, during early mouse development. Dev Dyn 206, 379-390.
Chen, Q., Khoury, M., Limmon, G., Choolani, M., Chan, J. K., and Chen, J. (2013). Human Fetal Hepatic Progenitor Cells Are Distinct from, but Closely Related to, Hematopoietic Stem/Progenitor Cells. Stem cells (Dayton, Ohio).
Cheng, X., Ying, L., Lu, L., Galvão, A. M., Mills, J. A., Lin, H. C., Kotton, D. N., Shen, S. S., Nostro, M. C., Choi, J. K., et al. (2012). Self-renewing endodermal progenitor lines generated from human pluripotent stem cells. Cell Stem Cell 10, 371-384.
Chetty, S., Pagliuca, F. W., Honore, C., Kweudjeu, A., Rezania, A., and Melton, D. A. (2013). A simple tool to improve pluripotent stem cell differentiation. Nature Methods.
Cho, C. H.-H., Hannan, N. R.-F., Docherty, F. M., Docherty, H. M., Joåo Lima, M., Trotter, M. W. B., Docherty, K., and Vallier, L. (2012). Inhibition of activin/nodal signalling is necessary for pancreatic differentiation of human pluripotent stem cells. Diabetologia.
Chung, W.-S., Shin, C. H., and Stainier, D. Y. R. (2008). Bmp2 signaling regulates the hepatic versus pancreatic fate decision. Developmental cell 15, 738-748.
Ciruna, B. G., and Rossant, J. (1999). Expression of the T-box gene Eomesodermin during early mouse development. Mech Dev 81, 199-203.
Coffinier, C., Barra, J., Babinet, C., and Yaniv, M. (1999). Expression of the vHNF1/HNF1beta homeoprotein gene during mouse organogenesis. Mech Dev 89, 211-213.
Cohen, D. E., and Melton, D. (2011). Turning straw into gold: directing cell fate for regenerative medicine. Nature Reviews Genetics.
Costa, M., Dottori, M., Sourris, K., Jamshidi, P., Hatzistavrou, T., Davis, R., Azzola, L., Jackson, S., Lim, S. M., Pera, M., et al. (2007). A method for genetic modification of human embryonic stem cells using electroporation. Nature Protocols 2, 792-796.
Cserjesi, P., Brown, D., Lyons, G. E., and Olson, E. N. (1995). Expression of the novel basic helix-loop-helix gene eHAND in neural crest derivatives and extraembryonic membranes during mouse development. Developmental Biology 170, 664-678.
Damjanov, I., Damjanov, A., and Damsky, C. H. (1986). Developmentally regulated expression of the cell-cell adhesion glycoprotein cell-CAM 120/80 in peri-implantation mouse embryos and extraembryonic membranes. Dev Biol 116, 194-202.
D'Amour, K. A., Agulnick, A. D., Eliazer, S., Kelly, O. G., Kroon, E., and Baetge, E. E. (2005). Efficient differentiation of human embryonic stem cells to definitive endoderm. Nat Biotechnol 23, 1534-1541.
D'Amour, K. A., Agulnick, A. D., Eliazer, S., Kelly, O. G., Kroon, E., and Baetge, E. E. (2005). Efficient differentiation of human embryonic stem cells to definitive endoderm. Nat Biotechnol 23, 1534-1541.
Davidson, B. P., and Tam, P. P. (2000). The node of the mouse embryo. Curr Biol 10, R617-619.
Davis, R. P., Ng, E. S., Costa, M., Mossman, A. K., Sourris, K., Elefanty, A. G., and Stanley, E. G. (2008). Targeting a GFP reporter gene to the MIXL1 locus of human embryonic stem cells identifies human primitive streak-like cells and enables isolation of primitive hematopoietic precursors. Blood 111, 1876-1884.
Deutsch, G., Jung, J., Zheng, M., Lora, J., and Zaret, K. S. (2001). A bipotential precursor population for pancreas and liver within the embryonic endoderm. Development 128, 871-881.
Ding, J., Yang, L., Yan, Y. T., Chen, A., Desai, N., Wynshaw-Boris, A., and Shen, M. M. (1998). Cripto is required for correct orientation of the anterior-posterior axis in the mouse embryo. Nature 395, 702-707.
Dollé, P., Izpisúa-Belmonte, J. C., Boncinelli, E., and Duboule, D. (1991). The Hox-4.8 gene is localized at the 5′ extremity of the Hox-4 complex and is expressed in the most posterior parts of the body during development. Mech Dev 36, 3-13.
Dono, R., Scalera, L., Pacifico, F., Acampora, D., Persico, M. G., and Simeone, A. (1993). The murine cripto gene: expression during mesoderm induction and early heart morphogenesis. Development 118, 1157-1168.
Drukker, M., Tang, C., Ardehali, R., Rinkevich, Y., Seita, J., Lee, A. S., Mosley, A. R., Weissman, I. L., and Soen, Y. (2012). Isolation of primitive endoderm, mesoderm, vascular endothelial and trophoblast progenitors from human pluripotent stem cells. Nat Biotechnol.
Duncan, S. A., Manova, K., Chen, W. S., Hoodless, P., Weinstein, D. C., Bachvarova, R. F., and Darnell, J. E. (1994). Expression of transcription factor HNF-4 in the extraembryonic endoderm, gut, and nephrogenic tissue of the developing mouse embryo: HNF-4 is a marker for primary endoderm in the implanting blastocyst. Proc Natl Acad Sci USA 91, 7598-7602.
Dunn, N. R., Vincent, S. D., Oxburgh, L., Robertson, E. J., and Bikoff, E. K. (2004). Combinatorial activities of Smad2 and Smad3 regulate mesoderm formation and patterning in the mouse embryo. Development 131, 1717-1728.
Dush, M. K., and Martin, G. R. (1992). Analysis of mouse Evx genes: Evx-1 displays graded expression in the primitive streak. Developmental Biology 151, 273-287.
Ernst, J., Kheradpour, P., Mikkelsen, T. S., Shoresh, N., Ward, L. D., Epstein, C. B., Zhang, X., Wang, L., Issner, R., Coyne, M., et al. (2011). Mapping and analysis of chromatin state dynamics in nine human cell types. Nature 473, 43-49.
Gadue, P., Huber, T. L., Paddison, P. J., and Keller, G. M. (2006). Wnt and TGF-β signaling are required for the induction of an in vitro model of primitive streak formation using embryonic stem cells. Proc Natl Acad Sci USA 103, 16806-16811.
Gertow, K., Hirst, C. E., Yu, Q. C., Ng, E. S., Pereira, L. A., Davis, R. P., Stanley, E. G., and Elefanty, A. G. (2013). WNT3A Promotes Hematopoietic or. Mesenchymal Differentiation from hESCs Depending on the Time of Exposure. Stem Cell Reports 1, 53-65.
Gharbi, S. I., Zvelebil, M. J., Shuttleworth, S. J., Hancox, T., Saghir, N., Timms, J. F., and Waterfield, M. D. (2007). Exploring the specificity of the PI3K family inhibitor LY294002. Biochem J 404, 15-21.
Gifford, Casey A., Ziller, Michael J., Gu, H., Trapnell, C., Donaghey, J., Tsankov, A., Shalek, Alex K., Kelley, David R., Shishkin, Alexander A., Issner, R., et al. (2013). Transcriptional and Epigenetic Dynamics during Specification of Human. Embryonic Stem Cells. Cell, 1-28.
Goldman, O., Han, S., Sourrisseau, M., Dziedzic, N., Hamou, W., Corneo, B., D'souza, S., Sato, T., Kotton, Darrell N., Bissig, K.-D., et al. (2013). KDR Identifies a Conserved Human and Murine Hepatic Progenitor and Instructs Early Liver Development. Cell Stem Cell 12, 748-760.
Goulding, M. D., Chalepakis, G., Deutsch, U., Erselius, J. R., and Gruss, P. (1991). Pax-3, a novel murine DNA binding protein expressed during early neurogenesis. EMBO J 10, 1135-1147.
Graf, T., and Enver, T. (2009). Forcing cells to change lineages. Nature 462, 587-594.
Grapin-Botton, A., and Melton, D. A. (2000). Endoderm development: from patterning to organogenesis. Trends in Genetics 16, 124-130.
Green, M. D., Chen, A., Nostro, M.-C., D'Souza, S. L., Schaniel, C., Lemischka, I. R., Gouon-Evans, V., Keller, G., and Snoeck, H.-W. (2011). Generation of anterior foregut endoderm from human embryonic and induced pluripotent stem cells. Nat Biotechnol.
Gualdi, R., Bossard, P., Zheng, M., Hamada, Y., Coleman, J. R., and Zaret, K. S. (1996). Hepatic specification of the gut endoderm in vitro: cell signaling and transcriptional control. Genes & Development 10, 1670-1682.
Guz, Y., Montminy, M. R., Stein, R., Leonard, J., Gamer, L. W., Wright, C. V., and Teitelman, G. (1995). Expression of murine STF-1, a putative insulin gene transcription factor, in beta cells of pancreas, duodenal epithelium and pancreatic exocrine and endocrine progenitors during ontogeny. Development 121, 11-18.
Hansson, M., Olesen, D. R., Peterslund, J. M. L., Engberg, N., Kahn, M., Winzi, M., Klein, T., Maddox-Hyttel, P., and Serup, P. (2009). A late requirement for Wnt and FGF signaling during activin-induced formation of foregut endoderm from mouse embryonic stem cells. Developmental Biology 330, 286-304.
Hart, A. H., Hartley, L., Ibrahim, M., and Robb, L. (2004). Identification, cloning and expression analysis of the pluripotency promoting Nanog genes in mouse and human. Dev Dyn 230, 187-198.
Hawkins, R. D., Hon, G. C., Lee, L. K., Ngo, Q., Lister, R., Pelizzola, M., Edsall, L. E., Kuan, S., Luu, Y., Klugman, S., et al. (2010). Distinct epigenomic landscapes of pluripotent and lineage-committed human cells. Cell Stem Cell 6, 479-491.
Heinz, S., Benner; C., Spann, N., Bertolino, E., Lin, Y. C., Laslo, P., Cheng, J. X., Murre, C., Singh, H., and Glass, C. K. (2010). Simple combinations of lineage-determining transcription factors prime cis-regulatory elements required for macrophage and B cell identities. Mol Cell 38, 576-589.
Hou, J., Charters, A. M., Lee, S. C., Zhao, Y., Wu, M. K., Jones, S. J. M., Marra, M. A., and Hoodless, P. A. (2007). A systematic screen for genes expressed in definitive endoderm by Serial Analysis of Gene Expression (SAGE). BMC Dev Biol 7, 92.
Hu, G., Cui, K., Northrup, D., Liu, C., Wang, C., Tang, Q., Ge, K., Levens, D., Crane-Robinson, C., and Zhao, K. (2013). H2A.Z facilitates access of active and repressive complexes to chromatin in embryonic stem cell self-renewal and differentiation. Cell Stem Cell 12, 180-192.
Initiative, I. S. C., Amps, K., Andrews, P. W., Anyfantis, G., Armstrong, L., Avery, S., Baharvand, H., Baker, J., Baker, D., Munoz, M. B., et al. (2011). Screening ethnically diverse human embryonic stem cells identifies a chromosome 20 minimal amplicon conferring growth advantage. Nat Biotechnol 29, 1132-1144.
Jacquemin, P., Pierreux, C. E., Fierens, S., van Eyll, J. M., Lemaigre, F. P., and Rousseau, G. G. (2003). Cloning and embryonic expression pattern of the mouse Onecut transcription factor OC-2. Gene Expr Patterns 3, 639-644.
James, R. G., Kamei, C. N., Wang, Q., Jiang, R., and Schultheiss, T. M. (2006). Odd-skipped related 1 is required for development of the metanephric kidney and regulates formation and differentiation of kidney precursor cells. Development 133, 2995-3004.
Jiang, R., Lan, Y., Norton, C. R., Sundberg, J. P., and Gridley, T. (1998). The Slug gene is not essential for mesoderm or neural crest development in mice. Developmental Biology 198, 277-285.
Jiang, W., Zhang, D., Bursac, N., and Zhang, Y. (2013). WNT3 Is a Biomarker Capable of Predicting the Definitive Endoderm Differentiation Potential of hESCs. Stem Cell Reports 1, 46-52.
Jin, C., Zang, C., Wei, G., Cui, K., Peng, W., Zhao, K., and Felsenfeld, G. (2009). H3.3/H2A.Z double variant-containing nucleosomes mark ‘nucleosome-free regions’ of active promoters and other regulatory regions. Nat Genet 41, 941-945.
Jung, J., Zheng, M., Goldfarb, M., and Zaret, K. S. (1999). Initiation of mammalian liver development from endoderm by fibroblast growth factors. Science 284, 1998-2003.
Kamiya, A., Kinoshita, T., Ito, Y., Matsui, T., Morikawa, Y., Senba, E., Nakashima, K., Taga, T., Yoshida, K., Kishimoto, T., et al. (1999). Fetal liver development requires a paracrine action of oncostatin M through the gp130 signal transducer. EMBO J 18, 2127-2136.
Kanai-Azuma, M., Kanai, Y., Gad, J. M., Tajima, Y., Taya, C., Kurohmaru, M., Sanai, Y., Yonekawa, H., Yazaki, K., Tam, P. P. L., et al. (2002). Depletion of definitive gut endoderm in Sox17-null mutant mice. Development 129, 2367-2379.
Kim, S. W., Yoon, S.-J., Chuong, E., Oyolu, C., Wills, A. E., Gupta, R., and Baker, J. (2011). Chromatin and transcriptional signatures for Nodal signaling during endoderm formation in hESCs. Developmental Biology 357, 492-504.
Knight, Z. A., Gonzalez, B., Feldman, M. E., Zunder, E. R., Goldenberg, D. D., Williams, O., Loewith, R., Stokoe, D., Balla, A., Toth, B., et al. (2006). A pharmacological map of the PI3-K family defines a role for p110alpha in insulin signaling. Cell 125, 733-747.
Kroon, E., Martinson, L. A., Kadoya, K., Bang, A. G., Kelly, O. G., Eliazer, S., Young, H., Richardson, M., Smart, N. G., Cunningham, J., et al. (2008). Pancreatic endoderm derived from human embryonic stem cells generates glucose-responsive insulin-secreting cells in vivo. Nat Biotechnol 26, 443-452.
Kumar, V., Muratani, M., Rayan, N. A., Kraus, P., Lufkin, T., Ng, H. H., and Prabhakar, S. (2013). Uniform, optimal signal processing of mapped deep-sequencing data. Nature Biotechnology 31, 615-622.
Landry, C., Clotman, F., Hioki, T., Oda, H., Picard, J. J., Lemaigre, F. P., and Rousseau, G. G. (1997). HNF-6 is expressed in endoderm derivatives and nervous system of the mouse embryo and participates to the cross-regulatory network of liver-enriched transcription factors. Developmental Biology 192, 247-257.
Langmead, B., Trapnell, C., Pop, M., and Salzberg, S. L. (2009). Ultrafast and memory-efficient alignment of short DNA sequences to the human genome. Genome Biol 10, R25.
Lawson, K. A., Meneses, J. J., and Pedersen, R. A. (1991). Clonal analysis of epiblast fate during germ layer formation in the mouse embryo. Development 113, 891-911.
Levak-\check{S}vajger, B., and vajger, A. (1974). Investigation on the origin of the definitive endoderm in the rat embryo. J Embryol Exp Morphol 32, 445-459.
Li, H., Arber, S., Jessell, T. M., and Edlund, H. (1999). Selective agenesis of the dorsal pancreas in mice lacking homeobox gene H1xb9. Nature Genetics 23, 67-70.
Li, Z., Gadue, P., Chen, K., Jiao, Y., Tuteja, G., Schug, J., Li, W., and Kaestner, K. H. (2012). Foxa2 and H2A.Z Mediate Nucleosome Depletion during Embryonic Stem Cell Differentiation. Cell 151, 1608-1616.
Lints, T. J., Parsons, L. M., Hartley, L., Lyons, I., and Harvey, R. P. (1993). Nkx-2.5: a novel murine homeobox gene expressed in early heart progenitor cells and their myogenic descendants. Development 119, 969.
Liu, P., Wakamiya, M., Shea, M. J., Albrecht, U., Behringer, R. R., and Bradley, A. (1999). Requirement for Wnt3 in vertebrate axis formation. Nature Genetics 22, 361-365.
Loh, K. M., and Lim, B. (2011). A precarious balance: pluripotency factors as lineage specifiers. Cell Stem Cell 8, 363-369.
Lu, C. C., Robertson, E. J., and Brennan, J. (2004). The mouse frizzled receptor is expressed in anterior organizer tissues. Gene Expr Patterns 4, 569-572.
Mahlapuu, M., Ormestad, M., Enerbäck, S., and Carlsson, P. (2001). The forkhead transcription factor Foxf1 is required for differentiation of extra-embryonic and lateral plate mesoderm. Development 128, 155-166.
McGrath, K. E., Koniski, A. D., Maltby, K. M., McGann, J. K., and Palis, J. (1999). Embryonic expression and function of the chemokine SDF-1 and its receptor, CXCR4. Developmental Biology 213, 442-456.
McKnight, K., Wang, P., and Kim, S. K. (2010). Deconstructing pancreas development to reconstruct human islets from pluripotent stem cells. Cell Stem Cell 6, 300-308.
McLean, C. Y., Bristor, D., Hiller, M., Clarke, S. L., Schaar, B. T., Lowe, C. B., Wenger, A. M., and Bejerano, G. (2010). GREAT improves functional interpretation of cis-regulatory regions. Nat Biotechnol 28, 495-501.
McLean, C. Y., Bristor, D., Hiller, M., Clarke, S. L., Schaar, B. T., Lowe, C. B., Wenger, A. M., and Bejerano, G. (2010). GREAT improves functional interpretation of cis-regulatory regions. Nat Biotechnol 28, 495-501.
Mishina, Y., Suzuki, A., Ueno, N., and Behringer, R. R. (1995). Bmpr encodes a type I bone morphogenetic protein receptor that is essential for gastrulation during mouse embryogenesis. Genes & Development 9, 3027-3037.
Murry, C. E., and Keller, G. (2008). Differentiation of embryonic stem cells to clinically relevant populations: lessons from embryonic development. Cell 132, 661-680.
Neubüser, A., Koseki, H., and Balling, R. (1995). Characterization and developmental expression of Pax9, a paired-box-containing gene related to Pax1. Developmental Biology 170, 701-716.
Niederreither, K., Vermot, J., Le Roux, I., Schuhbaur, B., Chambon, P., and Dollé, P. (2003). The regional pattern of retinoic acid synthesis by RALDH2 is essential for the development of posterior pharyngeal arches and the enteric nervous system. Development 130, 2525-2534.
Nieto, M. A., Bennett, M. F., Sargent, M. G., and Wilkinson, D. G. (1992). Cloning and developmental expression of Sna, a murine homologue of the Drosophila snail gene. Development 116, 227-237.
Norris, D. P., Brennan, J., Bikoff, E. K., and Robertson, E. J. (2002). The Foxh1-dependent autoregulatory enhancer controls the level of Nodal signals in the mouse embryo. Development 129, 3455-3468.
Nostro, M. C., Sarangi, F., Ogawa, S., Holtzinger, A., Corneo, B., Li, X., Micallef, S. J., Park, I.-H., Basford, C., Wheeler, M. B., et al. (2011). Stage-specific signaling through TGFβ family members and WNT regulates patterning and pancreatic specification of human pluripotent stem cells. Development 138, 861-871.
Orr-Urtreger, A., Bedford, M. T., Do, M. S., Eisenbach, L., and Lonai, P. (1992). Developmental expression of the alpha receptor for platelet-derived growth factor, which is deleted in the embryonic lethal Patch mutation. Development 115, 289-303.
Ostuni, R., Piccolo, V., Barozzi, I., Polletti, S., Termanini, A., Bonifacio, S., Curina, A., Prosperini, E., Ghisletti, S., and Natoli, G. (2013). Latent enhancers activated by stimulation in differentiated cells. Cell 152, 157-171.
Pearce, J. J., and Evans, M. J. (1999). Mml, a mouse Mix-like gene expressed in the primitive streak. Mech Dev 87, 189-192.
Plusa, B., Piliszek, A., Frankenberg, S., Artus, J., and Hadjantonakis, A.-K. (2008). Distinct sequential cell behaviours direct primitive endoderm formation in the mouse blastocyst. Development 135, 3081-3091.
Rada-Iglesias, A., Bajpai, R., Prescott, S., Brugmann, S. A., Swigut, T., and Wysocka, J. (2012). Epigenomic annotation of enhancers predicts transcriptional regulators of human neural crest. Cell Stem Cell 11, 633-648.
Rada-Iglesias, A., Bajpai, R., Swigut, T., Brugmann, S. A., Flynn, R. A., and Wysocka, J. (2011). A unique chromatin signature uncovers early developmental enhancers in humans. Nature 470, 279-283.
Radice, G. L., Rayburn, H., Matsunami, H., Knudsen, K. A., Takeichi, M., and Hynes, R. O. (1997). Developmental defects in mouse embryos lacking N-cadherin. Developmental Biology 181, 64-78.
Ram, O., Goren, A., Amit, I., Shoresh, N., Yosef, N., Ernst, J., Kellis, M., Gymrek, M., Issner, R., Coyne, M., et al. (2011). Combinatorial patterning of chromatin regulators uncovered by genome wide location analysis in human cells. Cell 147, 1628-1639.
Rashid, S. T., Corbineau, S., Hannan, N., Marciniak, S. J., Miranda, E., Alexander, G., Huang-Doran, I., Griffin, J., Ahrlund-Richter, L., Skepper, J., et al. (2010). Modeling inherited metabolic disorders of the liver using human induced pluripotent stem cells. J Clin Invest 120, 3127-3136.
Rausa, F., Samadani, U., Ye, H., Lim, L., Fletcher, C. F., Jenkins, N. A., Copeland, N. G., and Costa, R. H. (1997). The cut-homeodomain transcriptional activator HNF-6 is coexpressed with its target gene HNF-3 beta in the developing murine liver and pancreas. Developmental Biology 192, 228-246.
Rezania, A., Bruin, J. E., Riedel, M. J., Mojibian, M., Asadi, A., Xu, J., Gauvin, R., Narayan, K., Karanu, F., O'Neil, J. J., et al. (2012). Maturation of human embryonic stem cell-derived pancreatic progenitors into functional islets capable of treating pre-existing diabetes in mice. Diabetes.
Saga, Y., Hata, N., Koseki, H., and Taketo, M. M. (1997). Mesp2: a novel mouse gene expressed in the presegmented mesoderm and essential for segmentation initiation. Genes & Development 11, 1827-1839.
Saga, Y., Miyagawa-Tomita, S., Takagi, A., Kitajima, S., Miyazaki, J.i., and Inoue, T. (1999). MesP1 is expressed in the heart precursor cells and required for the formation of a single heart tube. Development 126, 3437-3447.
Sasaki, H., and Hogan, B. L. (1993). Differential expression of multiple fork head related genes during gastrulation and axial pattern formation in the mouse embryo. Development 118, 47-59.
Schier, A. F., and Talbot, W. S. (2005). Molecular genetics of axis formation in zebrafish. Annu Rev Genet 39, 561-613.
Shaner, N. C., Campbell, R. E., Steinbach, P. A., Giepmans, B. N. G., Palmer, A. E., and Tsien, R. Y. (2004). Improved monomeric red, orange and yellow fluorescent proteins derived from Discosoma sp. red fluorescent protein. Nat Biotechnol 22, 1567-1572.
Shawlot, W., Deng, J. M., and Behringer, R. R. (1998). Expression of the mouse cerberus-related gene, Cerr1, suggests a role in anterior neural induction and somitogenesis. Proc Natl Acad Sci USA 95, 6198-6203
Sherwood, R. I., Chen, T.-Y. A., and Melton, D. A. (2009). Transcriptional dynamics of endodermal organ formation. Dev Dyn 238, 29-42
Sherwood, R. I., Maehr, R., Mazzoni, E. O., and Melton, D. A. (2011). Wnt signaling specifies and patterns intestinal endoderm. Mech Dev 128, 387-400.
Si-Tayeb, K., Noto, F. K., Nagaoka, M., Li, J., Battle, M. A., Duris, C., North, P. E., Dalton, S., and Duncan, S. A. (2010). Highly efficient generation of human hepatocyte-like cells from induced pluripotent stem cells. Hepatology 51, 297-305.
Smith, A. G. (2001). Embryo-derived stem cells: of mice and men. Annu Rev Cell Dev Biol 17, 435-462.
Spence, J. R., Mayhew, C. N., Rankin, S. A., Kuhar, M. F., Vallance, J. E., Tolle, K., Hoskins, E. E., Kalinichenko, V. V., Wells, S. I., Zorn, A. M., et al. (2011). Directed differentiation of human pluripotent stem cells into intestinal tissue in vitro. Nature 470, 105-109.
Stafford, D., and Prince, V. E. (2002). Retinoic acid signaling is required for a critical early step in zebrafish pancreatic development. Curr Biol 12, 1215-1220.
Stafford, D., and Prince, V. E. (2002). Retinoic acid signaling is required for a critical early step in zebrafish pancreatic development. Curr Biol 12, 1215-1220.
Sumi, T., Tsuneyoshi, N., Nakatsuji, N., and Suemori, H. (2008). Defining early lineage specification of human embryonic stem cells by the orchestrated balance of canonical Wnt/β-catenin, Activin/Nodal and BMP signaling. Development 135, 2969-2979.
\check{S}vajger, A., and Levak-\check{S}vajger, B. (1974). Regional developmental capacities of the rat embryonic endoderm at the head-fold stage. J Embryol Exp Morphol 32, 461-467.
Takebe, T., Sekine, K., Enomura, M., Koike, H., Kimura, M., Ogaeri, T., Zhang, R.-R., Ueno, Y., Zheng, Y.-W., Koike, N., et al. (2013). Vascularized and functional human liver from an iPSC-derived organ bud transplant. Nature 499, 481-484.
Tam, P. P., and Beddington, R. S. (1987). The formation of mesodermal tissues in the mouse embryo during gastrulation and early organogenesis. Development 99, 109-126.
Tam, P. P. L., and Loebel, D. A. F. (2007). Gene function in mouse embryogenesis: get set for gastrulation. Nature Reviews Genetics 8, 368-381.
Tang, C., Lee, A. S., Volkmer, J.-P., Sahoo, D., Nag, D., Mosley, A. R., Inlay, M. A., Ardehali, R., Chavez, S. L., Pera, R. R., et al. (2011). An antibody against SSEA-5 glycan on human pluripotent stem cells enables removal of teratoma-forming cells. Nat Biotechnol 29, 829-834
Teo, A. K. K., Arnold, S. J., Trotter, M. W. B., Brown, S., Ang, L. T., Chng, Z., Robertson, E. J., Dunn, N. R., and Vallier, L. (2011). Pluripotency factors regulate definitive endoderm specification through eomesodermin. Genes & Development 25, 238-250.
Thomas, P. Q., Brown, A., and Beddington, R. S. (1998). Hex: a homeobox gene revealing peri-implantation asymmetry in the mouse embryo and an early transient marker of endothelial cell precursors. Development 125, 85-94.
Thorvaldsdottir, H., Robinson, J. T., and Mesirov, J. P. (2012). Integrative Genomics Viewer (IGV): high-performance genomics data visualization and exploration. Brief Bioinform.
Tiso, N., Filippi, A., Pauls, S., Bortolussi, M., and Argenton, F. (2002). BMP signalling regulates anteroposterior endoderm patterning in zebrafish. Mech Dev 118, 29-37.
Touboul, T., Hannan, N. R. F., Corbineau, S., Martinez, A., Martinet, C., Branchereau, S., Mainot, S Strick-Marchand, H., Pedersen, R., Di Santo, J., et al. (2010). Generation of functional hepatocytes from human embryonic stem cells under chemically defined conditions that recapitulate liver development. Hepatology 51, 1754-1765.
Trapnell, C., Pachter, L., and Salzberg, S. L. (2009). TopHat: discovering splice junctions with RNA-Seq. Bioinformatics 25, 1105-1111.
Tzouanacou, E., Wegener, A., Wymeersch, F. J., Wilson, V., and Nicolas, J.-F. (2009). Redefining the progression of lineage segregations during mammalian embryogenesis by clonal analysis. Developmental Cell 17, 365-376.
Visel, A., Blow, M. J., Li, Z., Zhang, T., Akiyama, J. A., Holt, A., Plajzer-Frick, I., Shoukry, M., Wright, C., Chen, F., et al. (2009). ChIP-seq accurately predicts tissue-specific activity of enhancers. Nature 457, 854-858.
Vlahos, C. J., Matter, W. F., Hui, K. Y., and Brown, R. F. (1994). A specific inhibitor of phosphatidylinositol 3-kinase, 2-(4-morpholinyl)-8-phenyl-4H-1-benzopyran-4-one (LY294002). The Journal of biological chemistry 269, 5241-5248.
Waddington, C. H. (1940). Organisers and Genes (Cambridge, UK, Cambridge University Press).
Wandzioch, E., and Zaret, K. S. (2009). Dynamic signaling network for the specification of embryonic pancreas and liver progenitors. Science 324, 1707-1710.
Wang, Z., Oron, E., Nelson, B., Razis, S., and Ivanova, N. (2012). Distinct lineage specification roles for NANOG, OCT4, and SOX2 in human embryonic stem cells. Cell Stem Cell 10, 440-454.
Wilkinson, D. G., Bhatt, S., and Herrmann, B. G. (1990). Expression pattern of the mouse T gene and its role in mesoderm formation. Nature 343, 657-659.
Xie, R., Everett, L. J., Lim, H.-W., Patel, N. A., Schug, J., Kroon, E., Kelly, O. G., Wang, A., D'amour, K. A., Robins, A. J., et al. (2013). Dynamic chromatin remodeling mediated by polycomb proteins orchestrates pancreatic differentiation of human embryonic stem cells. Cell Stem Cell 12, 224-237.
Xie, W., Schultz, M. D., Lister, R., Hou, Z., Rajagopal, N., Ray, P., Whitaker, J. W., Tian, S., Hawkins, R. D., Leung, D., et al. (2013). Epigenomic analysis of multilineage differentiation of human embryonic stem cells. Cell 153, 1134-1148.
Xu, P.-X., Zheng, W., Laclef, C., Maire, P., Maas, R. L., Peters, H., and Xu, X. (2002). Eya1 is required for the morphogenesis of mammalian thymus, parathyroid and thyroid. Development 129, 3033-3044.
Yusa, K., Rashid, S. T., Strick-Marchand, H., Varela, I., Liu, P.-Q., Paschon, D. E., Miranda, E., Ordóñez, A., Hannan, N. R. F., Rouhani, F. J., et al. (2011). Targeted gene correction of al-antitrypsin deficiency in induced pluripotent stem cells. Nature 478, 391-394.
Zhang, X., Huang, C. T., Chen, J., Pankratz, M. T., Xi, J., Li, J., Yang, Y., Lavaute, T. M., Li, X.-J., Ayala, M., et al. (2010). Pax6 is a human neuroectoderm cell fate determinant. Cell Stem Cell 7, 90-100.
Zorn, A. M., and Wells, J. M. (2009). Vertebrate endoderm development and organ formation. Annu Rev Cell Dev Biol 25, 221-251.
Zwart, R., Broos, L., Grosveld, G., and Meijer, D. (1996). The restricted expression pattern of the POU factor Oct-6 during early development of the mouse nervous system. Mech Dev 54, 185-194.
Zhu, Y., van Essen, D., and Saccani, S. (2012). Cell-type-specific control of enhancer activity by H3K9 trimethylation. Molecular Cell 46, 408-423.

1.-56. (canceled)

57. A method of differentiating anterior primitive streak cells into definitive endoderm (DE) lineage, comprising contacting said anterior primitive streak cells with:
a) one or more activators of TGFβ/Nodal signaling; and
b) one or more inhibitors of BMP signaling or one or more inhibitors of Wnt signaling,
to produce cells of the definitive endoderm (DE) lineage.

58. The method as claimed in claim 57, wherein the one or more activators of TGFβ/Nodal signaling is selected from the group consisting of Activin A, TGFβ1, TGFβ2 and Nodal; optionally wherein the one or more inhibitors of BMP signaling is selected from the group consisting of DM3189/LDN-193189, noggin, chordin, dorsomorphin and DMH1; optionally wherein the cells of the APS lineage are contacted with about 100 ng/ml of Activin A and about 250 nM of LDN-193189; optionally wherein the cells of the APS lineage are contacted with about 1 ng/ml to 10 μg/ml of Activin A and about 1 nM to 100 nM of LDN-193189; optionally wherein the one or more inhibitors of Wnt signaling is selected from the group consisting of Iwp2, Dkk1, C-59, Iwr-1 and XAV-939; optionally wherein the cells of the APS lineage are contacted with about 1 ng/ml to about 10 μg/ml of Activin A and about 1 nM to about 100 mM of Iwp2 or about 1 nM to about 100 mM C-59; optionally wherein the cells of the APS lineage are contacted with about 100 ng/ml of Activin A and about 4 μM of Iwp2 or about 1 μM of C-59 or about 300 ng/ml Dkk1; optionally wherein the cells of the defined endoderm lineage comprise elevated endoderm gene expression and decreased pluripotency gene expression relative to undifferentiated cells; optionally wherein the cells of the defined endoderm lineage comprise significantly reduced mesoderm gene expression; optionally wherein the differentiation of cells from anterior primitive streak lineage into cells of definitive endoderm (DE) lineage is completed within 24 to 96 hours; optionally wherein said cells of the anterior primitive streak are obtained from stem cells by contacting said stem cells with:
a) one or more activators of TGFβ/Nodal signaling, and
b) one or more activators of Wnt signaling; or
c) one or more inhibitors of PI3K/mTOR signaling,
to produce cells of the anterior primitive streak lineage; optionally wherein said cells of the anterior primitive streak are obtained from stem cells by contacting said stem cells with:
a) one or more activators of TGFβ/Nodal signaling, and
b) one or more activators of Wnt signaling; and
c) one or more inhibitors of PI3K/mTOR signaling; optionally wherein the one or more activators of TGFβ/Nodal signaling is Activin A, TGFβ1, TGFβ2 or Nodal; optionally wherein the one or more activators of Wnt signaling are CHIR99201 or Wnt3a or other family members of the Wnt signaling pathway; optionally wherein the one or more inhibitors of PI3K/mTOR signaling are PI-103, PIK-90, GDC0941, or LY294002; optionally wherein the one or more inhibitors of PI3K/mTOR signaling is PI-103; optionally wherein the stem cells are contacted with Activin A, PI-103 and CHIR99201; optionally wherein the stem cells are contacted with about 1 ng/ml to 100 μg/ml of Activin A, about 1 nM to 100 mM of PI-103 and about 1 nM to 100 mM of CHIR99201; optionally wherein the stem cells are contacted with about 100 ng/ml of Activin A, about 50 nM of PI-103 and about 2 μM of CHIR99201; optionally wherein the cells of the anterior primitive streak lineage comprise elevated gene expression of anterior streak or pan-streak markers and decreased expression of posterior streak markers relative to undifferentiated cells; optionally wherein the differentiation of stem cells to cells of the APS lineage is completed within 24 to 60 hours.

59. (canceled)

60. A method of differentiating cells of the DE lineage into cells of the posterior foregut (PFG), by contacting said cells of the DE with retinoic acid, a BMP inhibitor, a Wnt inhibitor and a FGF/MAPK inhibitor.

61. The method as claimed in claim 60, wherein the cells of the DE are contacted with about 1 nM to 100 mM retinoic acid, about 1 nM to 100 mM of LDN193189, about 1 nM to 100 mM of IWP2 and about 1 nM to 100 mM of PD0325901, optionally wherein the cells of the DE are contacted with about 2 μM retinoic acid, about 250 nM of LDN193189, about 4 μM of IWP2 and about 0.5 μM of PD0325901, optionally wherein the cells of the posterior foregut comprise elevated levels of posterior foregut gene expression without either MHG or AFG gene expression relative to undifferentiated cells; optionally wherein the DE cells are obtained using the method of claim 57.

62. A method of differentiating cells of the DE lineage into cells of the midgut/hindgut (MHG), by contacting said DE cells with a BMP activator, a Wnt activator and FGF activator.

63. The method as claimed in claim 62, wherein the cells of the DE are contacted with about 1 ng/ml to 100 μg/ml of BMP4, about 1 ng/ml to 100 μg/ml of FGF2, and about 1 nM to 100 μM of CHIR99201; optionally wherein the cells of the DE are contacted with about 10 ng/ml of BMP4, about 100 ng/ml of FGF2, and about 3 μM of CHIR99201; optionally wherein the cells of the MHG comprise elevated expression levels of MHG genes relative to undifferentiated cells; optionally wherein the differentiation is completed within 24 to 240 hours; optionally wherein the DE cells are obtained using the method of claim 57.

64. A method of inducing pancreatic progenitors of the PFG from the DE within three days by contacting said PFG with:
a) one or more FGF/MAPK inhibitors;
b) one or more BMP inhibitors; and
c) retinoic acid (RA).

65. A method of inducing liver progenitors of the PFG from the DE within four days by contacting said PFG with:
a) one or more TGFβ inhibitors;
b) retinoic acid (RA);
c) one or more BMP activators, and
d) one or more Wnt inhibitors.

66. A cell culture medium for differentiating a stem cell into definitive endoderm comprising one or more of the following factors: one or more activators of TGFβ/Nodal signaling, one or more activators of Wnt signaling; one or more inhibitors of PI3K/mTOR signaling; one or more activators of TGFβ/Nodal signaling; one or more inhibitors of BMP signaling or one or more inhibitors of Wnt signaling.

67. (canceled)

68. A cell culture medium for differentiating cells of the DE lineage into cells of the posterior foregut (PFG) comprising one or more of the following factors: retinoic acid, a BMP inhibitor, a Wnt inhibitor or a FGF/MAPK inhibitor.

69. A cell culture medium for differentiating cells of the DE lineage into cells of the midgut/hindgut (MHG), comprising one or more of the following factors: BMP activator, a Wnt activator or FGF activator.

70. A cell culture medium for inducing pancreatic progenitors of the PFG from the DE comprising one or more of the following factors: a) one or more FGF/MAPK inhibitors; b) one or more BMP inhibitors; or c) retinoic acid (RA).

71. A cell culture medium for inducing liver progenitors of the PFG from the DE comprising one or more of the following factors: a) one or more TGF inhibitors; b) one or more BMP activators, c) retinoic acid and d) one or more Wnt inhibitors.

72. (canceled)

73. A cell produced according to the method of claim 57.

74. A kit when used in the method of claim 57, comprising one or more containers of cell culture medium as claimed in claim 66, together with instructions for use.

75. The method of claim 64, further comprising contacting said PFG with one or more Hedgehog inhibitors; optionally further comprising contacting said PFG with one or more Wnt inhibitors; optionally further comprising contacting said PFG with Activin A; optionally further comprising contacting said PFG with one or more Hedgehog inhibitors; one or more WNT inhibitors and Activin A; optionally wherein said PFG is contacted with about 1 nM to 100 mM of PD0325901 or PD173074, about 1 nM to 100 mM of SANT 1, about 1 nM to 100 mM of LDN193189, about 1 nM to 100 mM of IWP2 or C59, about 1 nM to 100 mM of retinoic acid and about 1 ng/ml to 100 μg/ml of Activin A; optionally wherein said PFG is contacted with about 0.5 μM of PD0325901 or 100 nM of PD173074, about 150 nM of SANT 1, about 250 nM of LDN193189, about 4 μM of IWP2, about 2 μM of retinoic acid and about 10 ng/ml of Activin A; optionally wherein the cells of the pancreatic progenitors comprise elevated expression levels of pancreatic genes relative to undifferentiated cells and exclude hepatic progenitor gene expression; optionally further comprising contacting said PFG with about 1 ng/ml to 100 μg/ml of FGF2; optionally wherein the PFG is contacted with about 10 to 20 ng/ml of FGF2; optionally wherein the DE cells are obtained using the method of claim 57.

76. The method of claim 65, wherein said PFG is contacted with about 1 nM to 100 mM of A83-01, about 1 nM to 100 mM of RA, about 1 ng/ml to 10 μg/ml of BMP4, and about 1 nM to 100 mM of IWP2 or C59; optionally wherein said PFG is contacted with about 1 μM of A83-01, about 2 μM of RA, about 10 ng/ml of BMP4, and about 4 μM of IWP2; optionally wherein the cells of the liver progenitors comprise elevated expression levels of hepatic genes relative to undifferentiated cells and exclude pancreatic progenitor gene expression; optionally wherein the DE cells are obtained using the method of claim 57.


SEQUENCE LISTING (114 sequences)

SEQ ID NO: 1 - DNA, 20 bp, Artificial Sequence
  A synthetic oligonucleotide
ggagccatgtctggtaacgg

SEQ ID NO: 2 - DNA, 21 bp, Artificial Sequence
  A synthetic oligonucleotide
ccacgcgaatcactctcatct

SEQ ID NO: 3 - DNA, 20 bp, Artificial Sequence
  A synthetic oligonucleotide
agtgagaggcaacctggaga

SEQ ID NO: 4 - DNA, 20 bp, Artificial Sequence
  A synthetic oligonucleotide
acactcggaccacatccttc

SEQ ID NO: 5 - DNA, 19 bp, Artificial Sequence
  A synthetic oligonucleotide
tggacagttacgcgcacat

SEQ ID NO: 6 - DNA, 21 bp, Artificial Sequence
  A synthetic oligonucleotide
cgagtaggacatgctgtaggt

SEQ ID NO: 7 - DNA, 23 bp, Artificial Sequence
  A synthetic oligonucleotide
tgaccgctgtgacccgaaagact

SEQ ID NO: 8 - DNA, 22 bp, Artificial Sequence
  A synthetic oligonucleotide
agtgcgcagggcagcaagagta

SEQ ID NO: 9 - DNA, 20 bp, Artificial Sequence
  A synthetic oligonucleotide
tgcttccctgagacccagtt

SEQ ID NO: 10 - DNA, 25 bp, Artificial Sequence
  A synthetic oligonucleotide
gatcacttctttcctttgcatcaag

SEQ ID NO: 11 - DNA, 23 bp, Artificial Sequence
  A synthetic oligonucleotide
caacataaacggactcaatccca

SEQ ID NO: 12 - DNA, 22 bp, Artificial Sequence
  A synthetic oligonucleotide
accacctctacgaacacattgt

SEQ ID NO: 13 - DNA, 20 bp, Artificial Sequence
  A synthetic oligonucleotide
ggtaccccgacatccacttg

SEQ ID NO: 14 - DNA, 20 bp, Artificial Sequence
  A synthetic oligonucleotide
taatctccggcctagccaaa

SEQ ID NO: 15 - DNA, 21 bp, Artificial Sequence
  A synthetic oligonucleotide
tgacttcggcgtgttagtgtc

SEQ ID NO: 16 - DNA, 21 bp, Artificial Sequence
  A synthetic oligonucleotide
atgtggtccaggatagtcgtg

SEQ ID NO: 17 - DNA, 20 bp, Artificial Sequence
  A synthetic oligonucleotide
catcctggaccgctttctct

SEQ ID NO: 18 - DNA, 20 bp, Artificial Sequence
  A synthetic oligonucleotide
cacatcatgcaggtgaagca

SEQ ID NO: 19 - DNA, 20 bp, Artificial Sequence
  A synthetic oligonucleotide
aagtaccaaccccgcataca

SEQ ID NO: 20 - DNA, 20 bp, Artificial Sequence
  A synthetic oligonucleotide
taggctgtcacggagatgaa

SEQ ID NO: 21 - DNA, 20 bp, Artificial Sequence
  A synthetic oligonucleotide
agcttgggtgcctccttatt

SEQ ID NO: 22 - DNA, 20 bp, Artificial Sequence
  A synthetic oligonucleotide
tgcttccctgaaagacatca

SEQ ID NO: 23 - DNA, 21 bp, Artificial Sequence
  A synthetic oligonucleotide
aaggcatacgaacaggcactg

SEQ ID NO: 24 - DNA, 21 bp, Artificial Sequence
  A synthetic oligonucleotide
tacacaccttggtagtacgcc

SEQ ID NO: 25 - DNA, 18 bp, Artificial Sequence
  A synthetic oligonucleotide
gggagcggtgaagatgga

SEQ ID NO: 26 - DNA, 22 bp, Artificial Sequence
  A synthetic oligonucleotide
tcatgttgctcacggaggagta

SEQ ID NO: 27 - DNA, 23 bp, Artificial Sequence
  A synthetic oligonucleotide
gaggagaaagtggaggtctggtt

SEQ ID NO: 28 - DNA, 21 bp, Artificial Sequence
  A synthetic oligonucleotide
ctctgatgaggaccgcttctg

SEQ ID NO: 29 - DNA, 20 bp, Artificial Sequence
  A synthetic oligonucleotide
cacccgacgcccttttacat

SEQ ID NO: 30 - DNA, 19 bp, Artificial Sequence
  A synthetic oligonucleotide
gaaggctggatggatcggc

SEQ ID NO: 31 - DNA, 22 bp, Artificial Sequence
  A synthetic oligonucleotide
atcggctacaactacacctaca

SEQ ID NO: 32 - DNA, 21 bp, Artificial Sequence
  A synthetic oligonucleotide
gtacatgctgcacaggaagaa

SEQ ID NO: 33 - DNA, 21 bp, Artificial Sequence
  A synthetic oligonucleotide
agtgaccagatgcgtcgttac

SEQ ID NO: 34 - DNA, 19 bp, Artificial Sequence
  A synthetic oligonucleotide
tggtttccggcaggtttag

SEQ ID NO: 35 - DNA, 20 bp, Artificial Sequence
  A synthetic oligonucleotide
gaagtggttccttggcagac

SEQ ID NO: 36 - DNA, 20 bp, Artificial Sequence
  A synthetic oligonucleotide
tcctgcttgcctcaaagtgt

SEQ ID NO: 37 - DNA, 19 bp, Artificial Sequence
  A synthetic oligonucleotide
caccgcatctggagaacca

SEQ ID NO: 38 - DNA, 21 bp, Artificial Sequence
  A synthetic oligonucleotide
gcccatttcctcggtgtagtt

SEQ ID NO: 39 - DNA, 20 bp, Artificial Sequence
  A synthetic oligonucleotide
ttctcagggggtcatcttgc

SEQ ID NO: 40 - DNA, 20 bp, Artificial Sequence
  A synthetic oligonucleotide
atgaacagacccgcatttcc

SEQ ID NO: 41 - DNA, 20 bp, Artificial Sequence
  A synthetic oligonucleotide
cgcacggaatttgaacagta

SEQ ID NO: 42 - DNA, 20 bp, Artificial Sequence
  A synthetic oligonucleotide
ggatcagggacctgtcacac

SEQ ID NO: 43 - DNA, 26 bp, Artificial Sequence
  A synthetic oligonucleotide
agattatatcaggttgtacgggatca

SEQ ID NO: 44 - DNA, 20 bp, Artificial Sequence
  A synthetic oligonucleotide
acacagcggaaacactcgat

SEQ ID NO: 45 - DNA, 19 bp, Artificial Sequence
  A synthetic oligonucleotide
caagtgtgcgtctgccttt

SEQ ID NO: 46 - DNA, 22 bp, Artificial Sequence
  A synthetic oligonucleotide
cagctctttcttttcggctcta

SEQ ID NO: 47 - DNA, 24 bp, Artificial Sequence
  A synthetic oligonucleotide
actcggtgcgggagatgttcgagt

SEQ ID NO: 48 - DNA, 24 bp, Artificial Sequence
  A synthetic oligonucleotide
aaagctccggacgtgcggtacaga

SEQ ID NO: 49 - DNA, 22 bp, Artificial Sequence
  A synthetic oligonucleotide
agcagccgtatctgcaccagaa

SEQ ID NO: 50 - DNA, 22 bp, Artificial Sequence
  A synthetic oligonucleotide
ctcctttcggtcacacatgctg

SEQ ID NO: 51 - DNA, 21 bp, Artificial Sequence
  A synthetic oligonucleotide
ctccgcacctgctgggacttc

SEQ ID NO: 52 - DNA, 22 bp, Artificial Sequence
  A synthetic oligonucleotide
ctccacttccaaggcactacag

SEQ ID NO: 53 - DNA, 21 bp, Artificial Sequence
  A synthetic oligonucleotide
gtgcgtcctttaatcctcttc

SEQ ID NO: 54 - DNA, 19 bp, Artificial Sequence
  A synthetic oligonucleotide
gtgagagcaagcggaaaag

SEQ ID NO: 55 - DNA, 22 bp, Artificial Sequence
  A synthetic oligonucleotide
atctgcggcaaggcgttttcca

SEQ ID NO: 56 - DNA, 21 bp, Artificial Sequence
  A synthetic oligonucleotide
agccctcagatttgacctgtc

SEQ ID NO: 57 - DNA, 27 bp, Artificial Sequence
  A synthetic oligonucleotide
taagatgcccgacttcaactcccaggc

SEQ ID NO: 58 - DNA, 24 bp, Artificial Sequence
  A synthetic oligonucleotide
tgggccgcgacaggtacttgttga

SEQ ID NO: 59 - DNA, 22 bp, Artificial Sequence
  A synthetic oligonucleotide
ggaagcactgtttgccaagacc

SEQ ID NO: 60 - DNA, 22 bp, Artificial Sequence
  A synthetic oligonucleotide
ctgttgttggcggcacttagct

SEQ ID NO: 61 - DNA, 24 bp, Artificial Sequence
  A synthetic oligonucleotide
tggttatgttgctggacatgggtg

SEQ ID NO: 62 - DNA, 25 bp, Artificial Sequence
  A synthetic oligonucleotide
ggaagccgtgacagaatgactacct

SEQ ID NO: 63 - DNA, 21 bp, Artificial Sequence
  A synthetic oligonucleotide
cggctcctacgactattgccc

SEQ ID NO: 64 - DNA, 23 bp, Artificial Sequence
  A synthetic oligonucleotide
ggaacgtattccttgcttgccct

SEQ ID NO: 65 - DNA, 26 bp, Artificial Sequence
  A synthetic oligonucleotide
cagctcaccaactactccttccttca

SEQ ID NO: 66 - DNA, 22 bp, Artificial Sequence
  A synthetic oligonucleotide
tgcaacgcgctgaaaccataca

SEQ ID NO: 67 - DNA, 20 bp, Artificial Sequence
  A synthetic oligonucleotide
cccaccgacaagatgctcac

SEQ ID NO: 68 - DNA, 23 bp, Artificial Sequence
  A synthetic oligonucleotide
gccctgaattacttccattgctg

SEQ ID NO: 69 - DNA, 20 bp, Artificial Sequence
  A synthetic oligonucleotide
aggccacaatctcctctcac

SEQ ID NO: 70 - DNA, 21 bp, Artificial Sequence
  A synthetic oligonucleotide
ttgctggggattatggtggga

SEQ ID NO: 71 - DNA, 21 bp, Artificial Sequence
  A synthetic oligonucleotide
catggccaagattgacaacct

SEQ ID NO: 72 - DNA, 22 bp, Artificial Sequence
  A synthetic oligonucleotide
ttcccatatgttcctgcatcag

SEQ ID NO: 73 - DNA, 20 bp, Artificial Sequence
  A synthetic oligonucleotide
ctttgggctgctcgctatga

SEQ ID NO: 74 - DNA, 23 bp, Artificial Sequence
  A synthetic oligonucleotide
gcatgttgatttaacaagctgct

SEQ ID NO: 75 - DNA, 22 bp, Artificial Sequence
  A synthetic oligonucleotide
accccacacgcctttggcacaa

SEQ ID NO: 76 - DNA, 24 bp, Artificial Sequence
  A synthetic oligonucleotide
cacacccctggaataagccgagct

SEQ ID NO: 77 - DNA, 24 bp, Artificial Sequence
  A synthetic oligonucleotide
gctgggagcagccatcacagaagt

SEQ ID NO: 78 - DNA, 24 bp, Artificial Sequence
  A synthetic oligonucleotide
cacttggattcaccggtgcccgta

SEQ ID NO: 79 - DNA, 21 bp, Artificial Sequence
  A synthetic oligonucleotide
cgtgagaaggagggtctcttg

SEQ ID NO: 80 - DNA, 21 bp, Artificial Sequence
  A synthetic oligonucleotide
gtgggaggtagtcagagtgtc

SEQ ID NO: 81 - DNA, 22 bp, Artificial Sequence
  A synthetic oligonucleotide
agcagctccagctcaggcgaaa

SEQ ID NO: 82 - DNA, 20 bp, Artificial Sequence
  A synthetic oligonucleotide
tggcgctcagtgaggttcag

SEQ ID NO: 83 - DNA, 21 bp, Artificial Sequence
  A synthetic oligonucleotide
gttccctccatgcgaggaata

SEQ ID NO: 84 - DNA, 21 bp, Artificial Sequence
  A synthetic oligonucleotide
gctgggtaggtaatcgctctg

SEQ ID NO: 85 - DNA, 20 bp, Artificial Sequence
  A synthetic oligonucleotide
gcagagccccaatatccctg

SEQ ID NO: 86 - DNA, 23 bp, Artificial Sequence
  A synthetic oligonucleotide
ccgatccatagttcccacaagtt

SEQ ID NO: 87 - DNA, 22 bp, Artificial Sequence
  A synthetic oligonucleotide
tcctatttcgtgaactccacct

SEQ ID NO: 88 - DNA, 19 bp, Artificial Sequence
  A synthetic oligonucleotide
cgcggggtaatgtctcagc

SEQ ID NO: 89 - DNA, 26 bp, Artificial Sequence
  A synthetic oligonucleotide
acccctggatgcagcgaatgaattcg

SEQ ID NO: 90 - DNA, 25 bp, Artificial Sequence
  A synthetic oligonucleotide
gttccagggtctggtaccgcgagta

SEQ ID NO: 91 - DNA, 20 bp, Artificial Sequence
  A synthetic oligonucleotide
gaccccggcaatttctacgg

SEQ ID NO: 92 - DNA, 19 bp, Artificial Sequence
  A synthetic oligonucleotide
cgcaccgaataggctctgg

SEQ ID NO: 93 - DNA, 24 bp, Artificial Sequence
  A synthetic oligonucleotide
accagccacaggggtcccactttt

SEQ ID NO: 94 - DNA, 22 bp, Artificial Sequence
  A synthetic oligonucleotide
acgccgccgcttgtccttgtta

SEQ ID NO: 95 - DNA, 20 bp, Artificial Sequence
  A synthetic oligonucleotide
gggctctctgagaggcaggt

SEQ ID NO: 96 - DNA, 19 bp, Artificial Sequence
  A synthetic oligonucleotide
cctttgctctgcggttctg

SEQ ID NO: 97 - DNA, 26 bp, Artificial Sequence
  A synthetic oligonucleotide
ccgtgggcacgctctttactccatgt

SEQ ID NO: 98 - DNA, 27 bp, Artificial Sequence
  A synthetic oligonucleotide
ggattaggctcagccctgtgagaagac

SEQ ID NO: 99 - DNA, 24 bp, Artificial Sequence
  A synthetic oligonucleotide
ccgaccccaatcggaagcctaact

SEQ ID NO: 100 - DNA, 26 bp, Artificial Sequence
  A synthetic oligonucleotide
agtcccagatgagcattggcagcgag

SEQ ID NO: 101 - DNA, 23 bp, Artificial Sequence
  A synthetic oligonucleotide
gcgttgtttgtggctgttgcgca

SEQ ID NO: 102 - DNA, 24 bp, Artificial Sequence
  A synthetic oligonucleotide
agcttccccgctgtgtgtgttagg

SEQ ID NO: 103 - DNA, 21 bp, Artificial Sequence
  A synthetic oligonucleotide
gcagatgcaaaagtccaggtg

SEQ ID NO: 104 - DNA, 22 bp, Artificial Sequence
  A synthetic oligonucleotide
caggttgcgaagaactctgttt

SEQ ID NO: 105 - DNA, 22 bp, Artificial Sequence
  A synthetic oligonucleotide
cagaaggagaagcgcatgaccc

SEQ ID NO: 106 - DNA, 19 bp, Artificial Sequence
  A synthetic oligonucleotide
ctagctccccaggcgcgta

SEQ ID NO: 107 - DNA, 19 bp, Artificial Sequence
  A synthetic oligonucleotide
acgccgagctcagcaagat

SEQ ID NO: 108 - DNA, 20 bp, Artificial Sequence
  A synthetic oligonucleotide
tccacgtacggcctcttctg

SEQ ID NO: 109 - DNA, 24 bp, Artificial Sequence
  A synthetic oligonucleotide
cgatctttgcgcagagggtgctgt

SEQ ID NO: 110 - DNA, 22 bp, Artificial Sequence
  A synthetic oligonucleotide
tttgcacgctgccaggcgtaag

SEQ ID NO: 111 - DNA, 20 bp, Artificial Sequence
  A synthetic oligonucleotide
agcccttactgcccccagag

SEQ ID NO: 112 - DNA, 20 bp, Artificial Sequence
  A synthetic oligonucleotide
gggaagataccgggggacac

SEQ ID NO: 113 - DNA, 20 bp, Artificial Sequence
  A synthetic oligonucleotide
caacggggactgcacagatg

SEQ ID NO: 114 - DNA, 20 bp, Artificial Sequence
  A synthetic oligonucleotide
tgtttggcctggcgttcttt